SEQUENCE LISTING (127 sequences)

SEQ ID NO: 1 - DNA, 17021 bp, Homo sapiens
agcactttgggaagccaaggcaggtggattgcttgagctcaggagtttgaggccaccctgggcaacgtggcaaaatccagtctctacaaaaaacacaaaaattagctgggcacagtggtgcgcacctgctgtcccagctactcgggaggctgaggtgggaggatcacctgagcctgggaggtcaaggttgcagcaaaccaagatcctgccactgcactccagcctgggcaacagagcaaaaccctgtcaaaaaaaaaaaaaaaatctggtctaagcaccacctttgatggaggcaggaatcctgcagcagcctggagccaacccaatggctccctgcctctggtgctctatttcttatcaccatacaggtctctaaggtcacctttaggtctaacattgtatgaatgattgtcagcaactttccaatttttttcatatacatatattttttttattttattttttactttttctttttttgagacaggatctcactctgtcacccaggctggagtgcagtggcgtgatcatagctcactgtagcttcaacctcctgggcacaagtgatcctcccacctcagcttccaaggtacctgtgactacaggaatgcgccaccatgcccggctaattttgttatatatatatatatatatatttgtaccttggttttctcatctgtaacaccaggggttgaagtgctgtgatattttttggttctaggataatttggaaatagggatctcttaacatcttggatactgccaatagatcattcacgaaatccctttagatgagtgttatgtttacttaggtctctaaaacaatttgtaaggtcaacagatggggataagaatgacctgaagctggtcgtctttgttcgcaaccagaaacagtcctgcctcaaaaaaaaaaaaaaaaaaaagggagtaggcaggtggccatatttgtttctggcaagtgagtgctgaaggaaaggagctgaagcctcccacagtcataactggtgctggcaggctactgtctcggtcttgggcgccactgatctaaggtcacggctctgcttgctgctcccacccgctccagtttaaaacctgcggttccagggttctccagcccctccctttttcacgctccgaagccgagaaggcccaaagcgaagacagagaggacccggaagtagggaaaacctctgagcacgtgatgggggaacacgcgggtgctgtcacgtgatccgacaaacggcctctgcatagtgcagaacattctgctgctcttaaaggtacaggcctcagggtccctgctgtagacggggcgggggagagtacgatgggtggggcgtggtgggtcgtagggcgctcgagatggagcccccagcttccttgatggatcgcggggcgcgagtgccctagacaagccggagctgggaccggcaatcgggcgttgatccttgtcacctgtcgcagaccctcatccctcccgtgggagccccctttggacactctatgaccctggaccctcgggggacctgaacttgatgcgatgggaggctgtgcaggctcgcggcggcgcttttcggattccgagggtgagtattcccgcccaccctcatggaacgaccacctctggctcgcagcccacctgaggggaatgagagctgactcggccccgggaggacacgcaaggagcggtcgtgcacctgagagtaggtgggggtcatgccctcttctcgctctcccaggggaggagaccgtcccggagccccggctccctctgttggaccatcagggcgcgcattggaagaacgcggtgggcttctggtgagtggcctgagacttcagcgagtgacaatggcatgagaagagggaagtgaccggaggaaagggggaagaaagagttaagctgcgcaaaggagctgaacccacaaatatttactgaatccactggtttgccccaggggcagcatgtaaaatttccaaacttcactccttatcaccttccggatttagcaagatcctcatacctaccacctgtaggattatttacttttttcttccagccaattcagccttaacgttttttagtgcacatatttttaaaaagaaaactttaaatcacgactccacttggaaaaccagcgttttcctagtgaacgtttaaataaatgcatcactactaaaacttttttatttttttaatttattttattttatcttatttttttttctttttgagacagggtcttgctctgtttcccaggttggaatgcagtagtgcaatcacagctccctgcagcctggaactcctgggctcaagtgattctcccacctcagcctccaagtaggtgtggctacaggtgtgcaccaccgcatctgcctaatttttatatttttttatagagatgggggtctcactatgggacctgggctggtctcaaactcgtggcctcaagtgatcctcctacctcaaccttccaaagtgctgagattacaagcatgagccaccatgccgggcctgaacctttttttttttttttttttttgagacggagtctctctctgtcctccaggctggagtgcagtggcgcaatctcggctcactgcaaactccgcctcccaggttcacaccattcttttgcctcagcctccggagtagctgagactacaggcgcctgccaccacgcccggctatttttttgtatttttagtagagatggggtttcaccgcgttatccaggatggtctcgatctcctgacctcgtgatccgcccgcctcggcctcccaaagtgctgggattacaggtgtgagccaccgcgcccagctttttttttttttttttttaattttattttatttttgagacagagtctcactctatcacccaggctggagagcagtggcacaatctcggctcactgaaacctccacctccaaggttcaagctattctcctgtctcagctccccgagtagctgggattacaggtgcgtgccaccagacacagctaattttttgtatttttagtaaagacaggatttcaccatgttggtcaggctggtctagaactcctgaccttaggtgatccgcttgcctccacttcccaaagtgctgggattacaggcgtgagccaccgtgccccgccctgacataggggctttgggatcacagacttggattcacttccagcctccaaggcctctcccagaaactctctgtgaccactctccttttttttttttttttttgagatagagtctcgctcttgtagcccaggctggagtgcaatggcacaatctcggcttactgcaacctccgcctttcgggttcaagtggttctcctgcctcagcctcctgagtagctaggattacaggcatgtgcaacttcgcccagctaattttgtattttttagtagagacggggtttctccatattgctcaggctggtctcgaactcctgacctcaggcgatccgcccgcctcggcccctcaaagtgctggaattacaggtgtgagccactgcactcggcctcttcctttttattttttatttttgtgacagggtcttgctctgtcacccaggctagagtgcagtggcatgatcacagttcactgcagcctctgactcctggactcaagcaatcctcccacctcagcctcccaagtagctgggactacagtgcaagccaccacacctggctaatttttaaattttttgtagagatgggggtctcactatcttgcccaagcaccaaagcctgtatttttgaccccaacattgaatgaggatgggatctggtgatagagggaaaaaaaagagggggctgattgaagggcacaggtaagggaaggtttggcacaaaggctacagatggctgcaggatgaggagggagttgagacctgtcctgctccttccaggctgctgggcctttgcaacaacttctcttatgtggtgatgctgagtgccgcccacgacatccttagccacaagaggacatcgggaaaccagagccatgtaagtgactctctaccaccaccaccatggttagtccctgtgggaagatgagggggtgggacaaggtggggtaaagtttccagtctctggctgggcacagtggctcacacctgtaatcccagcagtttgggaggctgaggcgggcggatcacttgaagtcaggagttcgagaccagcctggccaacatggtgaaactccgactctactaaaaatacaaaaactacctgggtgtggtggcacacacctgtagtctgagctattcaggaggctgaggcaggagaattgcttgaagccaggaggtggaagttgcaatgagccaagatcacaccactgcactctagcttgggcaacagagtgagacaccatctcaaaaaaaagaaagctcttgaatgtgttatctaaacaaagtccatttacagggccatctgtggcctgtggtctagtagtgtgagactcatatggaaaccccctcttcattgtgcatgcagagacgctgaggtcctgagaagtcagtcactgctgacccaaagccatccttcaagtgaaggcagagctgggacgggagccaggctctgtgtgtctatccctctgcctccagggtgaaagacatgcttttctcccattaggtggacccaggcccaacgccgatcccccacaacagctcatcacgatttgactgcaactctgtctctacggctgtgcgtactcatctcacctggtccttgcctgacccagggcccctgggtctatagaccccactcccagcccttcactacccagctgggactgtcatgattaaaacagttgagacctgggctgggcgcactggctcacacttgtaatcccagcactttgggagaccaaggtgggaggatcacttgagcctaggagtttgagaccagcctgggcagtatagtgagacccccatcttaaagaaaaaaattaaaaaatatatatatataaaataattgagaccttaatataatttctgaggctgaggcggatggatcacttgagaccaggagttcaagaccagcctggccaacatggtgaaaccccatctgtactaaaaatacaaaaattagccaggcatggtggtgcacgcctgtaatcccagctactcaggaggctgaggcaggagaatcacttgaacccaggaggtggaggttgtagtgagccaagatcgagccactacactccagcctgggtggcagaatgagactcactctcaaaatatatatatatatagtttctgagtcctttctgtctgcaccatatattagctcatttaagccacacagcagtcctgtgagctaggtgctatgatattcccattttccagatgaggaaactgaagctcagagagtataaactcttgacacacaactaaggagtgggagagctgagacttgaacccaggcgtgcctgactccagagcctgtgtttgtagcaggcctgtttggccagctcctgcctctccttggccacgtggttgggagggttgtcccctggaagctctgcggtctcactctattctcctgtcccaggctgtgctcctggcggacatcctccccacactcgtcatcaaattgttggctcctcttggccttcacctgctgccctacaggtctgggtgagggtagtgggaggcagggtgggcaggagctgagaaaggggaggctgggatggctgagatgctgagagtagagaccgaccttccccctcccttcccttctcaccccctcagcccccgggttctcgtcagtgggatttgtgctgctggaagcttcgtcctggttgccttttctcattctgtggggaccagcctgtgtggtgagtgtgtggttctgtgtcagatggggagccccgaggaaccacatcagagcatttgtgggaagagtctccccagcctcccagaggaaagggattcattctgtcacccttagaagcctgctagggctatcagcagtaggcgatgggagactgggacaatttggaggggtaggcagtggaggagatgggagaaaatggatgaattagatggagattgaggtgaacaaagtcaagactctgtgatggaccaggcacagtgactcatgcctataatcccagcactttggaaagccaaggcaggcagatcacctgaggtcaggagttcgaaaccagcctggccaacatggagaaaccccgtctctaccaaaaatacaaaaattagctgggtgtggtggcaggagcctgtaatcccagctactcgggaggctgaggcaggagaatctcttgaacctgggaggcagaggttgcagtgagccgagatcacgccactgtactccagcctgggtgacagggcgagactccgtctccaaaaagaaaagaaaagaaaaagactgatgaaggggcagagacatcaagggtgcatgtctgcccctggtctgataactgggtggatggaggtgccactctccatgaagggacacgcaggggagtggggctctgcttcagacctggaacctggcctatgcatgggatctattggagcctctatgagctgatactgaggaggccatggccagacacattagaggcctgggcagtgtggcaaggtgtggtgtgaccatcccagtgcttgtcctccccccaggtgtggtcttcgctagcatctcatcaggccttggggaggtcaccttcctctccctcactgccttctaccccaggtaagcaggtggagcagggagtgtggggagaggctgtcccatggtcagcctaggtcctcctgaatgttcctgtgttctccttcccagggccgtgatctcctggtggtcctcagggactgggggagctgggctgctgggggccctgtcctacctgggcctcacccaggccggcctctcccctcagcagaccctgctgtccatgctgggtatccctgccctgctgctggccaggtgagctgccctgagccgggagggagaggggtccaaggagagaaaacttggccatggctgggtgtggtggctcacgcctgtaatcccagcactttgggaggccaaggagggcagatcgcctaaggtcaggaaaccagcctggccaacatggtgaaaccccgtctctactaaaaatacaacaattagccaggtgtggtggcgggtgcctgtagtcccaactactcaggaggctaaggcaggagaatcgcttgaaccccggaggcagtggtggcagtgagccgagatcgtgccattgcactccagcccgggcgacagagttagactctgtctcaggaagaaaaaaaaaaaaagaaagaaaagaaaacttgattatgattgcaatcttcaagtccctaccttgctgtgaagggaggcggaatctggactctgatagccccaggtgtgagtcctggagctgccactttttagctttgtagcgttgaacaagttactccacctctctgaaccctcagtttccccatatctcaaatggcagttgttcttgctttccttggaggtgatgagggtaatgcattcagcacagtgtggttcccaaggtgattagaagtaggatgagggtggactttatttgattagttccttttttttttttttttttttttaatcagtgttgaccaggttggcctcgaatgtgtagccttgcctgcctgagtgccagggcaacaggcctgaaccatggcgactcccttttttttttttttttttttttttttttgagacggagtctcatggtgtcactcaggctggagtgcagtgactggtgtgatctcagctcactgcagcctcggcctcccgggctctagtgattctcctgcctcagcctcccgagtagctgggactacaggcccatgccaccatgcctggctaattttttatatttttagtagagacggggtttcactgtattggccaggctggtctcaaactcctgacctcaagtgatccacctgccttggcctcccataatgctaggaatacaggcgtgagccaccgcgcccggcctgattagttctgtgtattttcatgcatattacaaaacactttggccgggcatggtggctcacatctgtaatcccagcactttgggaggccgaggcgggcgcaccacgaggtcaggagtttgagaccagcctggccaatatggtgaaaccccgtctctactaaaaatacaaaaattagccaggcttggtggcgcttgcctgtagtcccagctacttgggaggctgaggagaagaatcgcttgaacccaggaggcagaggttgcagtgagccgtgattgtgccactgcactccaggctgggcaacagagcgagactccgtctaaaaaaaaaaaaaacacacactttgaactagccagacacacctggcccttcacaagattagtgcttaggacaagtctaagtagaaaaagtgagtccatttaaagaaaaatattaagtaaaaatatatatatacaggaggaatatggatatggcaaataacatgaagccagaacctaagtgactaaaatcgaggaagttctggcctagcacagagtgaacgtataattaatgggagctagagcgaccattagaataataatggccagaaaacagaactgactcgctaggtatgagccagcagtccgaacaagggcctgctgagcacagtggctcacagctgtaatcccagtgctttaggaggctgaggctggaggaccactagaggctagaaatttgagaccagcctgggcaacatagcgagactccatttatacaaaaaatggaaaatatgaggcgggcttggtggcacgtgcctgtagtccccgctacttgggaggctgaggcgggaggattgcttgagcctgggaggtcgcggctgcagtgagctatgattgcaccactgcattccagcctggatgctggagaaagaccctgtctctgaaaaaaatcaaaacaaaatgaaggcccatgaataggaataaggagataaattcaggtccaaagaaagaagcctttgtccacaattggagctctcccgcacagcgtaggagccttagaggcagtgagctacccatctttgaaagtgttcaaaggtagaggtgttccctgggaaaagtggcctagatggtccctggggaccttcccagccagtagggttttctgaccctgccttcatcctactcctagctatttcttgttgctcacatctcctgaggcccaggaccctggaggggaagaagaagcagagagcgcagcccggcagcccctcataagaaccgaggccccggagtcgaagccaggtaggagacacagaccctcagagaggtcactttctttctctctgggtttggccttttcctctctgcaataggcaaagttaagaggggaaagagagtgagtgtactggttatgggaaaagcctttgtctatttgagaaatacttgttgaggacgagcacagtggctcacgcctgtaatatcccagcactttgggaggctgaggcaggaggaccacttgagctcagaagtctgagtccagcctgggcaacagagcaagaccttgtcgcaaaaaaaaaaaaaaaaaaaaaaaaaagaaaagaaaggaaggaagggagggagagaagaagggatattaattgagtacttaccatgtgccaggcattccaatcacaagtgctgaggccctgcggtggggataagctgggatgttctagaaccagagaatggccagtgagactgggcagggtgggccaatggccatctgtggagctgtacaagagacattggccgggcgtggtggttcatacctgtaatcccagcactttgggaggctgaggcagatggatcatttgagatcaagagtttgagaccagtctggccaacatggtaaaaccccgtctctactaaaaataaaaaaattagccaggtgtggtggtgcatgcctgtagtcccagctacttgggaagctgaggcacgagaatcacttgaacccaggaggcagaggctgcagtgagctgagattgcaccactgcactgcagcctgggcaactgagtgagactctgtctcaaaaaataaaaaaaattaaaaatcaaagagacgtgaagaggtctcacctggttagcttttattttcatcatgataaagtatgtatattacttgaagtttaccattttattattttttatcttactttatttttttgaggtggagtttcgctcttgttgcccaggctggagtgcaatggcgcaatctcagctcactacaacctctgcctcctgggttcaagtgattctcctgcctcagccacccgagtagctgggactacagacacctgccaccacacatggctaatttttgtatttttagtagagatggggtttctccatgttggccagggtggtcttgaactcctgacctcaggtgatccgcccacctcagtctcccaaggtgctgggattacaagcatgagccactgcgcccagccattttaaccatttttaagtgtccaatccagtggcatggaagtgagttcccactgttgcgtggtctggttaattctgtcataccggtgcctctttgccgagtcttcagtgtgaaaacttcatctgcccttctctctctgttcccctgcaggctccagctccagcctctcccttcgggaaaggtggacagtgttcaaggttcggatgatggctggggtatgtccctgtggggcttgctcacctccaggccccccgcttatatcttctgccttttccagggtctgctgtggtacattgttcccttggtcgtagtttactttgccgagtatttcattaaccagggacttgtaagtgaggggtgctaggaggggtgtggaggtggcgattggggctgggacccacacagccccgtccatctcccctgtctggtatttgttgcagtttgaactcctctttttctggaacacttccctgagtcacgctcagcaataccgctggtaagaggagcgagggcagtgggctgggagggcgccgtggtgatgcagctgccctgcccagtaggcaccggggggagcgggatggtccctggaggagcctcctctcctccccccaaccctaacctcaggtaccagatgctgtaccaggctggcgtctttgcctcccgctcttctctccgctgctgtcgcatccgtttcacctgggccctggccctgctgcaggtaccaaacccctgcccctcacttcactcccaccttggctcccagcttggctcccagcttccccaaaccccctgcttcccactatcagtgggaagtgtaaatttttttttttttttttgagacagagtctcgctctgtcgtccaggctggagtgcagtagcgcaatcttggcttactgcaacctctgcctcccgggttcaagtgactctcctgcctcagtctcctgagtagctgggattacaggcgcccgccaccatacctggctaatttttgtatttttagtagagatgggattttgccatattggctggtcttgaactcttgacctcaggtgatctgccggcctcagcctcccaaagtgctgggattacagggaagtgtaaattgtacactatcatttcttagcacagggaaccaaagtccagcagagctccatgtaaaatgtatagcctcaggagccaagcaaaactgagcttcagtactcagccactgtgtgacttagggcaggtctcagaacctctctgagcctcaatttcctcatgtataaattgggtgtggccaatacctatttttcagggtagttgtaagaaatagagatgagagaaagaatcgaagaacaagattttgtagtgtgtgtgtgtgtgtgtgtgcgtgtgtgtgtgtgtgtgtgtgtgtgtgtgtgtgtgttttaagcattgggattgggccgggccaggtggctcatgcctgtaatcccagcactatgggaggctgaggcaggtggatcatttgagcttggtagttcaagtccagcctgggcaacatggcacaatcacatctctacaaaaattagccaggtgcagtggcgcacacctgtagtcttagctactgggaagctgaggtgggaggatcacttgaacccatgaggtcaaggctgcagtaaccaaggtcatgccactgcactccagcctaggcaatagagcgagaccctgtctcccagaaaaagaaaaaagaaaggaaaagtcggaaccttcctctgtcgcctaggctggagtacagtggcatgatcataggtcatttacctcaggcaaagagacaggctttcaaataatgtttctaactataagaaaatgtacagttcttttattctagatagttaattataatagaattggtgactttgaagtcagtgggttatttatcttgagggtggtgagcaaattcaactgcccttggcaaacagtagtttacagctttaagggcgcaaacacccttccttacaacagtctaataaaaggagtttcatataaccagcattcattgtttcaaggttacaaatccacagcccctgcaggtaactgttacaataaactgtcaactgtgacaggaaagtcttctgcctttgaaatttaggaacttggccagtcctggtggctcacacctgtaatcccagcactttgggaggccaaggcaggtgaatcacctgaggtcaggatcaggagtttgagaccagtctggccaacacagtgaaaccccgtctctattaaaaatgcaaaaattagttgggcgtggtggcacctgcctgtcatctcagctactcgctaggctgaggcaggagaattgcttgaactccggatatggaggttgcagtgagccgagattgcagtactgtactccagcctgggccacagagcgagactccatctccaaaaaaaaagacttaggaactcaattctgtgatagcaaaatacagaaatagagattatactttaggctaggtgttgtggctcacacctgtaatcccaatgcttttggaggctagggtgggaggatcactcgaagccaggagcttgagaccagactggacaacatagtgagaccccccacctctacaaaaaattaaaaaaaaaaataagccaggtgggagaattgcttgagcccagaagtttgagagagcagcctgggtaacatcacgagacctcatctctacaaaaaacaacaacagggtgtggtgacattcacctgtaatcccagctactcgggaggctgaggcaggaggatcgctttagcccaggagttccaggctgcagtgagccataatcatgccactgtaccccagcctgggtgagaccctatctcaaaaaaaaagattacactttcatattatggcaaaagcatgtaaatatcctccagcctccttccttatttatctgagcagtgctgcaagcccagggtttgtacccacaaaaccccaaatttataactcagggtgcttgtagataccactgaaacatgtagcttatggttttaagctacatgtttttagctataatgttttttgttttgttttgttttttgagatggagtctcgctctgttgcccaggctggaatgtagcggtgcaatcttggctcactgcaacccctgcctcccagattcaagcgattctcaagcctcagccttccgagtagctgggattacaggtgcgccaccacgcccagcaaattttttgttttgttttgttttgttttgttttttagagacagagttttgctcttgtagctcaggctagagtacaatggcgtgatcttggctcactgcaacctccgcctcccaggttcaggcgattcttctgcctcagcctcctgcgtagctgggattacaggcacgtgccaccacacctggctaatttttgtattttttagtagagatggggtttcgccatgttggccaggctggtcttgaactcctgacctcaggtgatccacccgccttggccttccaaagtgctgggattacaggtgtgagccaccacacctggctagaggtgtgtcctttttaatgctcatctttaagtctacctcagatttaatgaattaggacctctagggtaagccccgcttggggcatttgtacctagatccccaggcaattcttacatatacgaaaagtatgaaccgccacgaggaggcctacacactgcacaagaggcaatgtggcgtggtcagtgctgggaggatggcacctccttgagagactggggcacagaggtgggacctacaccctggggtggggatcgaagaggcttcctggccaggtgcggtggctcctgcctataatcccagcactttgggaggctgaggcaggggggcagcgactgcttgagaccaggagttcgagcccagctgaataggatgcccggctactcccaccttgttttaccatagtgaaccaacggtttttccaataggggcaattttgctgcccagcggatgtttggcaacgtctgcaggcatgctttggttgtcacaactgagggatgctatgggcatctagcgggcagaggccagggatcctgctaaatgcgctgccatatacaggacagccctcaccatgcagaacaaccagcaccaaacgccaagagtgctgcaattgagaaatcctggtttcgatggtcccacttcctttaagaagcctccatttttttttttttttttttttttttttttatgagacaggatcttgctctgtcacccaggctggagtgcagtggcacaatcatagcttactgcagcctccacctcctaggttcaagcaatcctcctgtctcagactcccgagtagctgggactgcaggtgcgtgccaccatgctcaactaatttttaaattttttgtagagacggagttttgttatgttgcccaggctggtctcgaactcctggcctcaagtgatcatcccaccttggtctcccaatgtgctggtattacctgcatgagccactgcaccaggctgaggttgagaattttttttttttttttttgggatggagtctctctctgttgcccaggctggagtgcagtgctgcgatcttggctcactgcagcctctgcctcccaggttcaagtgattctcctgcctcagccttccaagtagctgggactacagaaccactacacccagataatttttgtatttttagttgagatggggttttaccatgtttagtagagaccaggccaggctggtctcgaaatcctgacctcaggtgatctgcccacctcagcctcccaaagtgctgggattacaggtgtgagccaccacaccgggctggtgttgagattttatctagacctggcagccctctccttcaccagtaactcctaaaaccagggacccctggaggggaggccacctctccttccctgccccgccctggtcccaggcttagcctctccccctgttcacagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggaggtcagcattggccgggcaagggctgggggtggcctgtccagggacacccagggcagggatgtctgggactgaagcctcacccctgctctctgccctcccagaccagtgatgagcaccgggagtttgcaatggcggccacctgcatctctgacacactggggatctccctgtcggggctcctggctttgcctctgcatgacttcctctgccagctctcctgatactcgggatcctcaggacgcaggtcacattcacctgtgggcagagggacaggtcagacacccaggcccaccccagagaccctccatgaactgtgctcccagccttcccggcaggtctgggagtagggaagggctgaagccttgtttcccttgcaggggggccagccattgtctcccacttggggagtttcttcctggcatcatgccttctgaataaatgccgattttatccatggacttcttatatcgtttttgtctctaaaaagaaacttttattatgaaagtaatacatgcccactttcctatatatagacagcataaagaaggtatgtcagcagatttctcccttttttgtttgtttgtttgtttgtttgtttgttttgacagagtctcactctgtcacctaggctggagtgcaatggcgtgatcttggctcactgcaacctccacttcccggttcaagcaattctcctgcctcagcctcccgagtagctgggattacaggtggtcactaccatgtctggctaatttttatatttttagtacagacgacattttgccatgttggccaggctggtctcaaactcatgacctcaggtgatccgcccaccttggcctcccaaagtgctgagattacaggtgtgagccactgtacctggcctctgccatctttgaggccttacgtattcattcattcatgcattcattgtttgggacaatctgtaaacaatcatgttggacattctttccagcatgaaagttttcagagaagtggaaagaattgtgcagtgaatcatcctgagtatgcgccaccctgttctatgttaacattttgttacagttgttttatcatgtagtccagcccccatcaaatctatgtattcactgttccatcagtgagtagatagagctaatgaatggcagggttggcacctgaggcattttattttattatttttttttatttttagagacagggttgcactctgtcacccaggctgaagtacagtggcacagtcatggctcactgtagccttgacctcctagactcaaacaatcctcacaccttggcctcccaagcagctgggactgcaagtgcatgccaccaagcccagctaatttttttttttttttttgtagagacgcgatcttcctatgttacccaggctggtcttgaactcttgagctcaagcagtcctgccttggcctcccaaagtactgagattacaagcatgag

SEQ ID NO: 2 - DNA, 14881 bp, Mus musculus
tggacgtctgctaagggaccccgacaaggcctagcttgaataagtgggtacacttgtagaggcaggagattgaaacccaggaaggactgggctaggagtctggctgactgtaggaaagggagctcaggcttcaggagaggctgtaagggagaggaaaggtgagactacgaggaatggtggaagtttcgggtatctctctgcccctctcaccagtgctcccctcaaagcctgatgtagcccagggagacgaagccggagaaaatccatcctgcaggtctctgtgtgtccagaaagctcacaatgaccatccgtccccacccacgaagtcctccccagcacccaagacacttcctcttcccctgactctccctgacaaatctccaccccaggccctgtgcacctcccccctgctcatggatgacttcatgacaggcagggggatggcttaacagtgtccaggtgactcagactcgggtgcacatctggaactaaagggcagaggatcgggccagaaaggggaaagggaaaaaaaaagtggaaacaggagacctcagaattgggtatgggggagggggcattcggcaagtgcagcctgagtgactcaggtcttgtattcgcacctcagccatttccgcctcctccaagccacagccttcagtgagaggaagctagggtttgtggttcctgatttatttgggggagaagctgtagaagtgaggataaggccactggaagttctgggttcccattttcaacgccattaagatttttctggggatgaaaggtctggattggtgctcaatgcccacaccctcacctatggttattcttgtactgtggtcatgtgtcctcacagcttactgtgagttccttaaggacagaggctgcagaatttctagagactcgtttgggtaggaggatcctttttctcactgtttgagtgatctaggaagaacaccacaggagggcatatttcaaacggcaaatcttaatgacaggtggttgaaaagacccgggagaagtgggcagaggctgctggaatccatagcagaggaataggcttgatgaagaatggtcaagatgctgccctgccatcgagtggaaggtaagggaaggactccttatttggaactgtgaggtgacacattccaactatcctctcagcctcattctcctaacttccaattaggacaaacggctgacatcacagaccaggaaaaacacccatagcagctttctcagtgaacttagcacctttgctctatttcttactgctgagtctctgaagtcagctaaaagtctagcctctgttgcacagtggggcggagggaccttggttttctcttgtaaaaccaggggttggacaactttcgtggtatttgttgttctaggatagtaaggcaaaggggatttaaccgtaacatcgtgaatattgccacagataattcatagaatgctgttttgtgggtgtttatttttaatcgatctactctcagctacttcccagatgcttattataggtctttacaataggtctgtaaggacaacagataacacttgaggctggtcgcctttgcgctcaagcagaacttgtccttgggacgttgggtggggtagtgggacatccgcagcaaagtagccataattgtttcaggcaagtaagagctgcaggaaagaagctgaagccttccgcgccatgactagtactggcagatttggtcggcgtctagagcgccgcttttcatggctctacttgtggctcccttcgactccaattcaaaatcagtgcttccagcgttctctcgaactctccttcttttattctccgaagtttgagaagtccaaaggcgaaaaagagaaaaaaggacccggaagtaagaagcaaccctcaagcacgtgatggaaggctcacgggtactgtcacgtgacccccaacctgctcgtctaccttgcggagagttcagctgctcttaaaggtacaggcctgggggcgggggctttgctttagaggggcagggggcggggtcaggcgggtggggcgtgggtgaacacgagctggagatctggggatccttcgacggacatgtcgtgtatagcagacagcggaacctgggactgaccgcggggcattgatccttcgcacccacctgtcccagactttaatctgttttcttgaagctagctcggaacacacgctgactttgggccctttgggggacccgaactcaatgttatgggaagttctgcgggctcgtggaggcgccttgaggattctgagagtgagtgctctcatcggttgccttgggaatgaccacctctgcccgcggctctcccgaggagttgggatgctggccctgccccctcaacaccatgcgggataggcggggccttcgtgtacccgagtgagtggggggtcatgctttctctacctcccaggggaggagaccgactcagagccccaggcccctcggttggatagtcggagtgtcctttggaagaatgcagtgggtttctggtaagtggtctgagagttgtgcaaaagtaaaatgccagctgggcgtggtggcacacgcctttaatcccagcatttgggaggcagaggcaggcggatttctgagttcgaggccagcctggtctacagagtgagttccaggacagccagggctacacagagaaaccctgtctcgaaaaacaaacaaacaaacaaacaaacaaagtaaagtgcgattggagcaaagatgagagccaagttgtgaaaagcagttgaatacacagatacttaatcaactgtgaactttccaagttccattaaattcccaccttcacctattgatcagatcttagtatctgtagagttatttattaaatgttttcctccagctaactttgttttttttcctcttccttcctttctttctttctttctttcttcctcttcctcctcctcctcttcctcttctttttcttcttttttttggaaaggtctttaaaccctacaagcagtaagggcttactttgacctttctaatcctgcttttgccttccaggtgctgcagttacaggccctactgtagcacaatgcccagaacacccattgtttctgtttgcagtgcagggtattaaaagcaggtccttcccgggctcacgcactgttgtacaagtgagctctgtctccagccaatttgcttttgccaagttctgagcacagctctgggagatgaaaataagaacaatatgccctgctattattttggttcccttgagatcagcaggtgaaaatttgtctccgttttgcacaggaatacgctagatatttgcattgtaccacagtgtttccgttgatgagaaatgcccagggatcagaatcttagtgtaagcgtgggagaaatagtgcttaggaggacttggcacttggccatgagctggtctaatctcaaggcctgttttgatagtggagaaggaaaaaaaaaaggtgtatctttaaggactgtaagtggatagaaagccagacagtgaagatgtgtcctcatttctaggatcttgggtctttgcaacaatttctcatatgtggtgatgctgagcgctgcccatgacatcctcaagcaggagcaggcgtctggaaaccagagccatgtaagtaagcccctcttcaccaccaccataggtcagtaagtgagcccctctgtaccactaccataggtcagtccgcatggggaatactaagaacccactcagggccagaggacccctgaaaaactcctagagtagctctgggtgggattggggagctaaaggtcttagtctcccacatcctcatggcaggagggatcttaaagagagcaacgggtaccaggttggtgaactcacataactggctgggtaagaccttattttgtggtgtgttttgtcattgtaccctgttgacatttgaggtcaggtgactgttgtgagtctgtcctgcatgttgtagatgtttagtagcatcccatgccccttgtcaaccaaaagtgacagccaaaaatgtctccagatgttacaaatgctcctgccaggtaacattacccaggtggagagcctttggcggcagtgagctagagcacgcaggccctaagaggcgcagcctccactttgcaggttcactaggaaatttgacccaagctgatattttccagaaacccagatttctatatgaatgctcttaaacatggtgcatccatcttggggggctgtctttggcctctggcccaaagtttgagatgcttataaacactctcttcattgtggacattgaaaaactaaggccccctagaagccttgcctacccacagccatttgtgaagttaggacagaactggacctgggccttggttagcctcctgccgctttgtcggaaagaccctcttctctcccataggtggaaccaggcccaacacccacaccccacaacagctcatctcgatttgactgcaactccatctccacagctgtgagtgtccatgtggggaccccacgatccttgcctttgcaatctcgtttccccacccccacccccatccagttgggaatgttgggattgcagtgatgaatataaatatgtaaatagtaattattagttcaggtgccccccttcccccagctttgtgagtgaaacactgtaagtttccctctttggatgaggaaactgaaggtcagagtgtataaatatgccctgacagccatttgaggagccccagagcagggaatgaatggagatctatctaactccagagcctaacttacagcaggtctcactggccggctccccggctgggttgggaaggttgtctccgggaaggtctaggctctcactgtgttctcctgtctcaggcggtgctcctagcagacatccttcccacccttgtcatcaaactcctggcgcctcttggccttcacttgctgccttacaggtctgggtcggggtggcaggagggaggcagggtgggaggaagcgggagtctggagagtctcgcatggaggtggccttctgagttcctcctcatctcttctcagcccccgggtgctcgtcagtggagtttgttctgctgggagctttgttctggttgccttctctcagtcagtggggttaagcctgtgtggtgagtacgaagctgggagaggttgatggactcaacagagtgatgtgctgtagtctccccagcttcctgtgtgtgtgtgtgtgtgtgtgtgtgtgtgtgtgtgtgtgtgtgtgtgagagagagagagagagagagagagagagacacacacttgggagattgtgaatttgagattagttggggccacatagtgaattcaagcctatcctaagctgcacagcaaaaccctatctcaaaaaacaaagacaaaaaaaaaccaaaaaaacaaggagagggccagtccaggctgatttacatagcaagttccaggccagcctggactacatccccgagactttgtctcaaagacaaacctcctagagcttccctaaccagaagtgagctctaggaacaattggagggtgtcaataggaaggaatggggagactgagtataggtggggatgagcagggttgaataattgggaattattaactgaatgaagatgggggtacagtctcggagaggacatgtggaggagggtgtggcttttcagatgagaccctagcctgtctgttcagtgacagatctgttcttagccctgctgagctgatggagttgaggctgtaaacaaaccaccggggactgcggctttcagttaaaggtcactgtttgctgtttgtcctcccaggagtggttttggccagcatctcctcagggctaggggaggtcaccttcctctcactgactgccttctaccccaggtaagcggcctgggccaggaggactgggagacgagtgccccctgctggcttccatcttcctcagtgctcttgtcttcttttcagtgctgtgatctcatggtggtcttcgggtaccgggggtgcagggcttcttggatcgctgtcttacctgggactcacccaggctggcctctccccgcagcacaccctactttctatgttggggatccctgttctgctgctagccaggtgagtgtccttaggcccaaaggatgaaagatggaaggaggagcttgatgctaatccaatcttcagagctcttcagccttgttgtgaaggggtgtggactctggactctcgacacccctcaggtggaatctcagagtggggctgtgttagctgtgcgactctggacaacttcattcctgaccttcagttgtctctgtctcaggctggttctgtctcatccgcacaggtcggctcctgagactggggctgggactgagtgcaccccggtttatttcttctgtgtgtatttgtatattttagaaaacattattaagccaggtgtagtggtacatgcctttcatcccagcactctggaggcatcatgttatagcttagggctattgtggggtatccaccagaaagacggctcagacatgggtttaaacccagtggaaagccgattagtcacctggtaactacacactgcatgttcagtggcatttccattggagtcagttgtcctaaggtccggtggcctgttacaaggctatcttgaagaaaaggagtcagtatgtgtgtaatgctgaacacccacagtcctggctcttgggaggtgatggaaaaggctcatgtgtttgaagcctacctgggatacacgtggagaccctggctcaaaagctaactgagataattgaacatatggcaaatgcctctaatccaagccctcaggaggcaggaggatctctgtgagtttggggtcagcctggtctacatcttgagctccaggccagccagagctgcatagttaagaccctgtctcaaaaaaaaaaaaaaagaaagaaagaaataaagcaatcccatgatcatacagaccctgggtaaactcagtcacaacacgaaacaaaaagacactgagtatgagaaagggacctgtgatgtgggaaaggtcacagggtgtggggacccgagggggtggggagagtcaccagagggcagtatatatgtgaataaaaatgtcaagaaacacatttagtaagaatctctgtgaagtggtgaatagatgagaagtactgagaacagggaaacgagcaggtgctgtgactgagtcaaatgaggtccctgggtagaaacaaagaagcttttccagcacagggtacctagaggctgggagcaccccatgcaagtgtgaagggcctggaggggcgggcctgcaggacctctggggcctttcccgacaagagccccctgactcgcttctccattccatcccagctatttcttgttgctcacgtctcctgaacccctggaccctggaggggaaaacgaggcagagactgctgcccggcagcctctcataggcaccgagaccccagagtcaaagccaggtaggatgcaaaggccctcccatcccagggccactttccgccttggggttttggtctcaacagtggacacagttgagggaaacaggctgctaggtgtacaggtctgggtcaaagcagtcatgtatacttactaactactgactggatgccagacattccgattgctgggaatggtagtgctccagatgaaggaatgagagcattttcacaggacataaaactgcgatggggcccagaggggtggcgcacaccttgaatcccaacactccggaggcagccacaatcagatctctatgatttcaaggccggttgggctacatagtaagaccctgcacagacccagaaagtaacggttaaggaaatggggtggggatggctaagggagctgctttacaggaggttccaagtgtgggggttttgttgttgttttgttttttcttcaagtttttgcttattggtttttaaatagtctctctgtatagccctagctggcctagaaatcactgtgtactctcgaatggcctcaacctcacaaatccacctgcctctgcctctcaaatgctaggagtaaacgcatatgccaccatacacctggctaggatttatttttctatttattgatgtgtatgtgtatttgtgaacttataccgcggatgaccagcagagggcatcagatgccttggagttgcagttacaggcagttgggagctggccgatgttgccaggattcaaacgtggaccccctcatggttgagcaaccagtgctcgctcgctctttctttccttttctctctttctttctttctttctttctttctttctttctttctttctttctttctttctttctttctttctttctttcttccttccttccttccttcctttctttctttctttctttcggatttatttatttatttaatgtatatgagcacaatgtagttgtcttcagacacaccagaagagggcaagagggcatcagatctcataacagatggttgtgagccaccatgtggttgctgggaattgaactcaggacctctggaagagcagtcagtgctcttaaccactgagccatctctccagcccctctttcttttttttttattgtttgtttgttttgtttttgttttgttttttgtttttttgttgttgttgttgaggcagagcttctctgtgtagctctggctgtcctggaactcactctctagaccagaagtcaaaaattctcctgcctctgctttccatgcgctgggattcagggcgtgtgccgccacttaaccccaagccacctctccaactctgagaaaggctctacttatttatttaattttgtaattatttgtttgtttgtttgcttgcttttcgaggaagggtttctctgtgtagccctggctgtcctggaactcacactgtatgtaatccaggctgccctcaaactttcagagatccacctgcctcagcctcccaagtggggattaaaggcatgcgccaccactgcccagcgatttgtctatttttattttatgtgcattggtgttttgcctgtgtgcatgtctatgggagggtgttggatcccctggaactggagttacagacagttgtgagctgccacgtgggtgctgggaattgaggtccgctggaagagcagccattactcttaaccactgagccatctcttgagcccccgagagaacccccaacaacaaaccttttatttttgagaatggagctgtctgggcaagagcgtgtgcacaagtgtgaggtctgacgttctgaggattgagctctgctcattcagactcggcagtgagtgccctcatccaccgagcatccatcagcccaagacagccttttgaaggatatctctgggctgaaacttggctgtcaaggtgtcagctttgcaaatgcctaggtgatcatgttccaggcacaggaaacagctcagggtgaggccttatgtgagaaaaggagcctggttgttctagaaatgaaaggcttagccagagtctgggatggagtcggtagataaaagctgtaccctgatctatagtcttgctcattaaaaaaaaaaaaaaaaagttttataagtatatgtgtgtatatacatatgtatacagcgcgaagtttaccattttagcgactactaagtgtgcactaagtggcatttaaatccacacactgctgtgctggctggttgcttgctgcatgctggcaccttagctaagcgttcagcgtgcaacctcctccccttccccctgctctgtaggtgccagctgggacctctccctccaggaaaggtggacagtgttcaaggtttgaatggtggctgcgggtgccccgtgaggatcctctgcctcttgccttccacttacatctcccgactcttccagggtctcttgtggtacatcatccctctggtgctggtctactttgcagaatactttatcaaccagggacttgtgagtgaggggcgccggggagggatgtgggaggagagatagggctgaggttccgccatctccatctgtctggtcttcgctgcagttcgagctcctgtttttccggaacacttccctaagccatgctcagcagtaccgatggtgagagaagttggcaggtgggcagtaggctgggaggatccctgatgttggggtcctggggttgaccccaaaaagtgccctgagagcctcttcctcttctctgccccaaccttaggtaccagatgctataccaggctggtgtgttcgcctcccgctcttctctccaatgttgccgaatacggttcacctgggtcctagccctgctccaggtactgagtgctgccctgtttgttcacacccacctcggcctcctgagtttcttacattagtgtactccagcgttcctaagtacagcactaaaggccagctggactccatataaaatgtgtggtggcaccaggaggaaccagtcatacttgtgggtaaattccagtttctgttctcaagagctgtgtgactcagagcagtctctgaacctctattgagcctcagttttcctatcaggaagtcagatggggtggagcaggattgtgcacctagcctgtctgaggctgtgggtcccatccctagtactgcaaaaattaaaatatttaatagctgtgggcaacacctatttctcagtgtttgtatagcaatgttgagaaaaaaaaggaagaacaagatattgttttgatttattttggttttgttcttgtctgctttatttttttttctttgtttgtttttaaggtagggtctcactgtgtagtcctgggtggcctggaatccgatatgtagactaggctagcctgaagctcacagatctgcttcatgaatgcgggcgttgagggcctgtgcctcgtgcctggccgtcgtgctttgttctcacagtcgttctgactgcacctggggccttgttcctgctggtccatgctgattgttacgaaacttcttaagccctcaagcttgctttcatttacttacttatttactttaaagatttgtttgtttatatataaacaaactgtagctgtgttcagacacaccagaagagggcatcagctagccgggcgtggtggcgcacgcctttaatcccagcactcgggaggcagaggcaggcggatttctgagttcgaggccagcctggtctgaaaagtgagctccaggacagccagggctacacagagaaaccctgtctcgaacccccccccaaaaaaaaaaaacaaaaaaaaacaaaaaaaaaaaacagaagagggcatcagatcccatcatagatggttgtgagccaccatgtggttgctgggaatcgaactcaggacctctggcagagcagtcagtgctcttaaccgctgagccatcgctctagccctatttatttattttatacaatttaaataaacacacttatatttatatttatttattggtgggagggagacacttgccaagccgtgtgtatggaggtcagaggacagcgtgcaggattggttcccttcttcaacgtgtgggttctggtgatcaaactcaggtcatacaggcaagtacctgtagctgagccatctcttcagcccaaacagggtttctctgtatagccctggctgtcctggaactcactctgtggaccaggctggcctcgaactcagaaatctgcctgcctctgcctcccaagtgctgggtttaaaggcgtgcgccaccactgcccagctcagcccaaactttttatttggaggcaaagtcttcaaagctggccttgaacctacatctgaggcccacataagccttgaccattgtttagtttatatatacacaaacctttgtctatgtgtatatatgtgaaccacctgtgtgcctggtgcccatggaggccagagagggtatcagatccctggaactggaactacagataggtgtggaccgccctgtgggtgctgtggaagaacagcgggtgcctttaactactgagccatctttccagtccaaaattttgtactcttgacatttgcagctagatcattctgatgtgttacttgctgtaggtgtttagcagtatccctagctggtgagacggcaagtaggtctccagacattgtcaaatgacccattgatttcagtgcagtaattaggggagaacaggtgatgttcagggggtccttgggtgctgaggataggactctggtagagcatttgcctctgaatcacaagctctcgtgttagctctgcagtgtatgtgtgtgtgtgtgtgtgtgtgtgtgtgtgtgtgtgtgtgtgtgtttgtgtgtgtgatacacatggtaggggtgtctaggtgttaggattcatgccattaaaaaagaaacaaaaaggcaagaaggagaggaggtgactgatgaccacagtggaatgaccctggtccaactgagagctcaccagtgatgggaagttgaggaacattacaggctaagagtgacagggacaagtcaaggaaggggagaaacccacaaggcatgtgaggatgaggatggggtcatcgagggcctaccaagatgtcggggtcccacaggcccagctgggcactgggaccctacacagccctctgcttcagcggcatctcctataacctggcagctctgagacctgctgcctccctgccgtattgttgcccagtctcacgcctcccttccctctgcccacagtgcctcaacctggccctcctgctggcagatgtctgcttgaacttcttgcccagcatctacctcatcttcatcatcattctgtacgaagggctcctgggtggggccgcttacgtgaataccttccacaacattgctctggaggtctgtgccagttggacaagggctgtgggtggccttaggagccttgtggatggaagccctaacccttgttctttgctctcccagaccagtgacaagcaccgagagtttgccatggaagctgcctgtatctctgacaccttgggaatctccctgtcgggggtcctggccctgcctctgcatgacttcctctgtcacctcccttgacaggagttgctcgacacacactgatctgcaggcacatgagcagatcacacatcttcgagctctgccacagcctttccctgccccactgcagcaaggagcccctgatgtttcccactcctgagctggcctcagagttttctcctaccctctgcccttctaataaatgcttattttaacagtttcctttttgtgcagtctctgaaacggaaagtttacataagcccactgtgaaagatatatgtatttatgaatgtataaagtatttcagacaaggtgtggcacacacctgtaattccagcactcagcagactggagaatccaagttcacgtctgactgggatatacggtgagaccctgaggactagggctatagttcagtggtagagtacttgcccaggatgtcctgggctctcagttttatccctggaaaacaattcaaaagcagcgtatgctaatataagcctgtagtcccaggattggggaggtagagacaggaggatccagagttcatggtcctcagctacataggaaaccaaggtcagcctggcctacataagatcttgttttacaataattacattgtgtatttctggcttctttataaaaaaaaatcaaatgtccataggtatgtagatttatatctgggtcttcgattccattgatcaacttgtctgtttttatgccaatactgtgtgggttttattactataactctgtagtagtgcttgaaataaggatggtgatacctccaggagttctattactgtacagagttgttttagcaatcctgtgtttcttgtttttttcatatgaaattgagtattgttctttcaaggtctataaagaattgtgttggaattttgatggacaactgatttctttcttcaatgtcttaaagtttttatcatataactctttgacttgcttggttagattgaccctacaatattcatagaagacgaagtgggtagtagccctgaacgaatgtcacaggagacaacttcctgaacagaacaccaatagttcaggcactaaaattgacaataaaccagatctcatgaaactgaaaagcttttgtgaggcaaatgatattgtcatctggcagcctacagaatagaaaaagatttctaccaactcaatatctgatagaaggctaatatccgtaatatataaagaactcaaagccgggcggtggtggcgcacgcctttaatcctagcactcaggaggcagaggcaggtggatttctgagttcaaggccagcctggtctacaaagtgagttccaggacagccagagctacacagagaaaccctgtctcgaaaaaaaaacaaagaacaaaaaacacgagggagagagaaagagaaagagaactcaagaagctagacatcaataaggcaaatagcccaaataaaaaatggggtacagagctgaacagagacttctcaacaggaatctcaaatggctgagaaacacttagaacttcatcatccttatccagcagggaaatgcaaatcaaaaccactctgagattccatctttaacacctgtcaggatggctaagatcaaaactcaagtaacagctcatgctggcgaggatgtggagaaaggggagcacttgggagcaaggggagcactcttcggttgttggtgagaatgcaaatttgtacagccactttggagatcaatatggtagtttcttagaaaattggaacctccagacccagctataccacttctggggatatacccaaaggatgtcccatcctaccacagggacacttgctcaattatgttcacagcagctttatttataataaccagaaactggaaacaacctcgatgtcccttaactaaggaatggataagaaa

SEQ ID NO: 3 - DNA, 25 bp, Artificial sequence
  Synthetic oligonucleotide
acacagaaccacacactcaccacac

SEQ ID NO: 4 - DNA, 18 bp, Artificial sequence
  Synthetic oligonucleotide
gagaagagatgaggagga

SEQ ID NO: 5 - DNA, 18 bp, Artificial sequence
  Synthetic oligonucleotide
ggctgagaagagatgagg

SEQ ID NO: 6 - DNA, 18 bp, Artificial sequence
  Synthetic oligonucleotide
gggctgagaagagatgag

SEQ ID NO: 7 - DNA, 18 bp, Artificial sequence
  Synthetic oligonucleotide
cactgacgagcacccggg

SEQ ID NO: 8 - DNA, 18 bp, Artificial sequence
  Synthetic oligonucleotide
ccactgacgagcacccgg

SEQ ID NO: 9 - DNA, 18 bp, Artificial sequence
  Synthetic oligonucleotide
tccactgacgagcacccg

SEQ ID NO: 10 - DNA, 18 bp, Artificial sequence
  Synthetic oligonucleotide
aaactccactgacgagca

SEQ ID NO: 11 - DNA, 18 bp, Artificial sequence
  Synthetic oligonucleotide
gaacaaactccactgacg

SEQ ID NO: 12 - DNA, 18 bp, Artificial sequence
  Synthetic oligonucleotide
agcagaacaaactccact

SEQ ID NO: 13 - DNA, 18 bp, Artificial sequence
  Synthetic oligonucleotide
tcccagcagaacaaactc

SEQ ID NO: 14 - DNA, 18 bp, Artificial sequence
  Synthetic oligonucleotide
aagctcccagcagaacaa

SEQ ID NO: 15 - DNA, 18 bp, Artificial sequence
  Synthetic oligonucleotide
aacaaagctcccagcaga

SEQ ID NO: 16 - DNA, 18 bp, Artificial sequence
  Synthetic oligonucleotide
ccagaacaaagctcccag

SEQ ID NO: 17 - DNA, 18 bp, Artificial sequence
  Synthetic oligonucleotide
gcaaccagaacaaagctc

SEQ ID NO: 18 - DNA, 18 bp, Artificial sequence
  Synthetic oligonucleotide
gaaggcaaccagaacaaa

SEQ ID NO: 19 - DNA, 18 bp, Artificial sequence
  Synthetic oligonucleotide
gagagaaggcaaccagaa

SEQ ID NO: 20 - DNA, 18 bp, Artificial sequence
  Synthetic oligonucleotide
gactgagagaaggcaacc

SEQ ID NO: 21 - DNA, 18 bp, Artificial sequence
  Synthetic oligonucleotide
cactgactgagagaaggc

SEQ ID NO: 22 - DNA, 18 bp, Artificial sequence
  Synthetic oligonucleotide
accccactgactgagaga

SEQ ID NO: 23 - DNA, 18 bp, Artificial sequence
  Synthetic oligonucleotide
cttaaccccactgactga

SEQ ID NO: 24 - DNA, 18 bp, Artificial sequence
  Synthetic oligonucleotide
caggcttaaccccactga

SEQ ID NO: 25 - DNA, 18 bp, Artificial sequence
  Synthetic oligonucleotide
cacacaggcttaacccca

SEQ ID NO: 26 - DNA, 18 bp, Artificial sequence
  Synthetic oligonucleotide
tcaccacacaggcttaac

SEQ ID NO: 27 - DNA, 18 bp, Artificial sequence
  Synthetic oligonucleotide
gtactcaccacacaggct

SEQ ID NO: 28 - DNA, 18 bp, Artificial sequence
  Synthetic oligonucleotide
cttcgtactcaccacaca

SEQ ID NO: 29 - DNA, 18 bp, Artificial sequence
  Synthetic oligonucleotide
ccagcttcgtactcacca

SEQ ID NO: 30 - DNA, 18 bp, Artificial sequence
  Synthetic oligonucleotide
tctcccagcttcgtactc

SEQ ID NO: 31 - DNA, 18 bp, Artificial sequence
  Synthetic oligonucleotide
gggatggaatggagaagc

SEQ ID NO: 32 - DNA, 18 bp, Artificial sequence
  Synthetic oligonucleotide
agctgggatggaatggag

SEQ ID NO: 33 - DNA, 18 bp, Artificial sequence
  Synthetic oligonucleotide
aaatagctgggatggaat

SEQ ID NO: 34 - DNA, 18 bp, Artificial sequence
  Synthetic oligonucleotide
caagaaatagctgggatg

SEQ ID NO: 35 - DNA, 18 bp, Artificial sequence
  Synthetic oligonucleotide
gcaacaagaaatagctgg

SEQ ID NO: 36 - DNA, 18 bp, Artificial sequence
  Synthetic oligonucleotide
gtgagcaacaagaaatag

SEQ ID NO: 37 - DNA, 18 bp, Artificial sequence
  Synthetic oligonucleotide
agacgtgagcaacaagaa

SEQ ID NO: 38 - DNA, 18 bp, Artificial sequence
  Synthetic oligonucleotide
caggagacgtgagcaaca

SEQ ID NO: 39 - DNA, 18 bp, Artificial sequence
  Synthetic oligonucleotide
ggttcaggagacgtgagc

SEQ ID NO: 40 - DNA, 18 bp, Artificial sequence
  Synthetic oligonucleotide
gggttcaggagacgtgag

SEQ ID NO: 41 - DNA, 18 bp, Artificial sequence
  Synthetic oligonucleotide
cccctccagggtccaggg

SEQ ID NO: 42 - DNA, 18 bp, Artificial sequence
  Synthetic oligonucleotide
tttcccctccagggtcca

SEQ ID NO: 43 - DNA, 18 bp, Artificial sequence
  Synthetic oligonucleotide
tcgttttcccctccaggg

SEQ ID NO: 44 - DNA, 18 bp, Artificial sequence
  Synthetic oligonucleotide
tgcctcgttttcccctcc

SEQ ID NO: 45 - DNA, 18 bp, Artificial sequence
  Synthetic oligonucleotide
tctctgcctcgttttccc

SEQ ID NO: 46 - DNA, 18 bp, Artificial sequence
  Synthetic oligonucleotide
gcagtctctgcctcgttt

SEQ ID NO: 47 - DNA, 18 bp, Artificial sequence
  Synthetic oligonucleotide
ggcagcagtctctgcctc

SEQ ID NO: 48 - DNA, 18 bp, Artificial sequence
  Synthetic oligonucleotide
gccgggcagcagtctctg

SEQ ID NO: 49 - DNA, 18 bp, Artificial sequence
  Synthetic oligonucleotide
ggctgccgggcagcagtc

SEQ ID NO: 50 - DNA, 18 bp, Artificial sequence
  Synthetic oligonucleotide
gagaggctgccgggcagc

SEQ ID NO: 51 - DNA, 18 bp, Artificial sequence
  Synthetic oligonucleotide
ctatgagaggctgccggg

SEQ ID NO: 52 - DNA, 18 bp, Artificial sequence
  Synthetic oligonucleotide
gtgcctatgagaggctgc

SEQ ID NO: 53 - DNA, 18 bp, Artificial sequence
  Synthetic oligonucleotide
ctcggtgcctatgagagg

SEQ ID NO: 54 - DNA, 18 bp, Artificial sequence
  Synthetic oligonucleotide
gggtctcggtgcctatga

SEQ ID NO: 55 - DNA, 18 bp, Artificial sequence
  Synthetic oligonucleotide
cctggctttgactctggg

SEQ ID NO: 56 - DNA, 18 bp, Artificial sequence
  Synthetic oligonucleotide
cctacctggctttgactc

SEQ ID NO: 57 - DNA, 18 bp, Artificial sequence
  Synthetic oligonucleotide
gcatcctacctggctttg

SEQ ID NO: 58 - DNA, 18 bp, Artificial sequence
  Synthetic oligonucleotide
ctttgcatcctacctggc

SEQ ID NO: 59 - DNA, 18 bp, Artificial sequence
  Synthetic oligonucleotide
gggcctttgcatcctacc

SEQ ID NO: 60 - DNA, 18 bp, Artificial sequence
  Synthetic oligonucleotide
gggagggcctttgcatcc

SEQ ID NO: 61 - DNA, 18 bp, Artificial sequence
  Synthetic oligonucleotide
agctgatcatattctacc

SEQ ID NO: 62 - DNA, 25 bp, Artificial sequence
  Synthetic oligonucleotide
agctgatcatattctacctggtgct

SEQ ID NO: 63 - DNA, 18 bp, Artificial sequence
  Synthetic oligonucleotide
aaaggcaaccaggacgaa

SEQ ID NO: 64 - DNA, 18 bp, Artificial sequence
  Synthetic oligonucleotide
aaatgctctgatgtggtt

SEQ ID NO: 65 - DNA, 18 bp, Artificial sequence
  Synthetic oligonucleotide
acaaatcccactgacgag

SEQ ID NO: 66 - DNA, 18 bp, Artificial sequence
  Synthetic oligonucleotide
acaggctggtccccacag

SEQ ID NO: 67 - DNA, 18 bp, Artificial sequence
  Synthetic oligonucleotide
accacacaggctggtccc

SEQ ID NO: 68 - DNA, 18 bp, Artificial sequence
  Synthetic oligonucleotide
actctcagcatctcagcc

SEQ ID NO: 69 - DNA, 18 bp, Artificial sequence
  Synthetic oligonucleotide
aggacgaagcttccagca

SEQ ID NO: 70 - DNA, 18 bp, Artificial sequence
  Synthetic oligonucleotide
caaccaggacgaagcttc

SEQ ID NO: 71 - DNA, 18 bp, Artificial sequence
  Synthetic oligonucleotide
cactcaccacacaggctg

SEQ ID NO: 72 - DNA, 18 bp, Artificial sequence
  Synthetic oligonucleotide
cagaaccacacactcacc

SEQ ID NO: 73 - DNA, 18 bp, Artificial sequence
  Synthetic oligonucleotide
cagaatgagaaaaggcaa

SEQ ID NO: 74 - DNA, 18 bp, Artificial sequence
  Synthetic oligonucleotide
ccacacactcaccacaca

SEQ ID NO: 75 - DNA, 18 bp, Artificial sequence
  Synthetic oligonucleotide
ccatctgacacagaacca

SEQ ID NO: 76 - DNA, 18 bp, Artificial sequence
  Synthetic oligonucleotide
cccacaaatgctctgatg

SEQ ID NO: 77 - DNA, 18 bp, Artificial sequence
  Synthetic oligonucleotide
ccccacagaatgagaaaa

SEQ ID NO: 78 - DNA, 18 bp, Artificial sequence
  Synthetic oligonucleotide
ctctgatgtggttcctcg

SEQ ID NO: 79 - DNA, 18 bp, Artificial sequence
  Synthetic oligonucleotide
ctggtccccacagaatga

SEQ ID NO: 80 - DNA, 18 bp, Artificial sequence
  Synthetic oligonucleotide
gaagcttccagcagcaca

SEQ ID NO: 81 - DNA, 18 bp, Artificial sequence
  Synthetic oligonucleotide
gcagcacaaatcccactg

SEQ ID NO: 82 - DNA, 18 bp, Artificial sequence
  Synthetic oligonucleotide
gctccccatctgacacag

SEQ ID NO: 83 - DNA, 18 bp, Artificial sequence
  Synthetic oligonucleotide
ggaaggtcggtctctact

SEQ ID NO: 84 - DNA, 18 bp, Artificial sequence
  Synthetic oligonucleotide
ggtgagaagggaagggag

SEQ ID NO: 85 - DNA, 18 bp, Artificial sequence
  Synthetic oligonucleotide
gtcggtctctactctcag

SEQ ID NO: 86 - DNA, 18 bp, Artificial sequence
  Synthetic oligonucleotide
tcccactgacgagaaccc

SEQ ID NO: 87 - DNA, 18 bp, Artificial sequence
  Synthetic oligonucleotide
tctctactctcagcatct

SEQ ID NO: 88 - DNA, 18 bp, Artificial sequence
  Synthetic oligonucleotide
tgacacagaaccacacac

SEQ ID NO: 89 - DNA, 18 bp, Artificial sequence
  Synthetic oligonucleotide
tgagaaaaggcaaccagg

SEQ ID NO: 90 - DNA, 18 bp, Artificial sequence
  Synthetic oligonucleotide
ttccagcagcacaaatcc

SEQ ID NO: 91 - DNA, 18 bp, Artificial sequence
  Synthetic oligonucleotide
gggaaggtccccagggac

SEQ ID NO: 92 - DNA, 18 bp, Artificial sequence
  Synthetic oligonucleotide
tggctgggaaggtcccca

SEQ ID NO: 93 - DNA, 18 bp, Artificial sequence
  Synthetic oligonucleotide
cctactggctgggaaggt

SEQ ID NO: 94 - DNA, 18 bp, Artificial sequence
  Synthetic oligonucleotide
aaaaccctactggctggg

SEQ ID NO: 95 - DNA, 18 bp, Artificial sequence
  Synthetic oligonucleotide
gtcagaaaaccctactgg

SEQ ID NO: 96 - DNA, 18 bp, Artificial sequence
  Synthetic oligonucleotide
gcagggtcagaaaaccct

SEQ ID NO: 97 - DNA, 18 bp, Artificial sequence
  Synthetic oligonucleotide
tgaaggcagggtcagaaa

SEQ ID NO: 98 - DNA, 18 bp, Artificial sequence
  Synthetic oligonucleotide
taggatgaaggcagggtc

SEQ ID NO: 99 - DNA, 18 bp, Artificial sequence
  Synthetic oligonucleotide
aggagtaggatgaaggca

SEQ ID NO: 100 - DNA, 18 bp, Artificial sequence
  Synthetic oligonucleotide
tagctaggagtaggatga

SEQ ID NO: 101 - DNA, 18 bp, Artificial sequence
  Synthetic oligonucleotide
agaaatagctaggagtag

SEQ ID NO: 102 - DNA, 18 bp, Artificial sequence
  Synthetic oligonucleotide
caacaagaaatagctagg

SEQ ID NO: 103 - DNA, 18 bp, Artificial sequence
  Synthetic oligonucleotide
gagatgtgagcaacaaga

SEQ ID NO: 104 - DNA, 18 bp, Artificial sequence
  Synthetic oligonucleotide
ctcaggagatgtgagcaa

SEQ ID NO: 105 - DNA, 18 bp, Artificial sequence
  Synthetic oligonucleotide
tgggcctcaggagatgtg

SEQ ID NO: 106 - DNA, 18 bp, Artificial sequence
  Synthetic oligonucleotide
ggtcctgggcctcaggag

SEQ ID NO: 107 - DNA, 18 bp, Artificial sequence
  Synthetic oligonucleotide
tccagggtcctgggcctc

SEQ ID NO: 108 - DNA, 18 bp, Artificial sequence
  Synthetic oligonucleotide
tcccctccagggtcctgg

SEQ ID NO: 109 - DNA, 18 bp, Artificial sequence
  Synthetic oligonucleotide
cttcttcccctccagggt

SEQ ID NO: 110 - DNA, 18 bp, Artificial sequence
  Synthetic oligonucleotide
tgcttcttcttcccctcc

SEQ ID NO: 111 - DNA, 18 bp, Artificial sequence
  Synthetic oligonucleotide
ctctctgcttcttcttcc

SEQ ID NO: 112 - DNA, 18 bp, Artificial sequence
  Synthetic oligonucleotide
ctgcgctctctgcttctt

SEQ ID NO: 113 - DNA, 18 bp, Artificial sequence
  Synthetic oligonucleotide
ccgggctgcgctctctgc

SEQ ID NO: 114 - DNA, 18 bp, Artificial sequence
  Synthetic oligonucleotide
ggctgccgggctgcgctc

SEQ ID NO: 115 - DNA, 18 bp, Artificial sequence
  Synthetic oligonucleotide
gggcctcggttcttatga

SEQ ID NO: 116 - DNA, 18 bp, Artificial sequence
  Synthetic oligonucleotide
cctacctggcttcgactc

SEQ ID NO: 117 - DNA, 18 bp, Artificial sequence
  Synthetic oligonucleotide
tgtctcctacctggcttc

SEQ ID NO: 118 - DNA, 18 bp, Artificial sequence
  Synthetic oligonucleotide
gtctgtgtctcctacctg

SEQ ID NO: 119 - DNA, 18 bp, Artificial sequence
  Synthetic oligonucleotide
tgagggtctgtgtctcct

SEQ ID NO: 120 - DNA, 18 bp, Artificial sequence
  Synthetic oligonucleotide
ctctctgagggtctgtgt

SEQ ID NO: 121 - DNA, 18 bp, Artificial sequence
  Synthetic oligonucleotide
gtgacctctctgagggtc

SEQ ID NO: 122 - DNA, 18 bp, Artificial sequence
  Synthetic oligonucleotide
agaaagtgacctctctga

SEQ ID NO: 123 - DNA, 18 bp, Artificial sequence
  Synthetic oligonucleotide
gagaaagaaagtgacctc

SEQ ID NO: 124 - DNA, 18 bp, Artificial sequence
  Synthetic oligonucleotide
ccagagagaaagaaagtg

SEQ ID NO: 125 - DNA, 18 bp, Artificial sequence
  Synthetic oligonucleotide
caaacccagagagaaaga

SEQ ID NO: 126 - DNA, 18 bp, Artificial sequence
  Synthetic oligonucleotide
aaggccaaacccagagag

SEQ ID NO: 127 - DNA, 18 bp, Artificial sequence
  Synthetic oligonucleotide
aggaaaaggccaaaccca

(19) United States
(12) Patent Application Publication
(10) Pub. No.: US 2017/0268000 A1
(43) Pub. Date: Sep. 21, 2017

(54) ANTISENSE COMPOUNDS AND USES THEREOF

(71) Applicants: IONIS PHARMACEUTICALS ,INC., Carlsbad, CA (US); ROSALIND FRANKLIN UNIVERSITY OF MEDICINE AND SCIENCE, North Chicago, IL (US)

(72) Inventors: Frank RIGO, Carlsbad, CA (US); Michelle L. HASTINGS, North Chicago, IL (US)

(73) Assignees: Ionis Pharmaceuticals, Inc., Carlsbad, CA (US); Rosalind Franklin University of Medicine and Science, North Chicago, CA (US)

(21) Appl. No.: 15/039,188

(22) PCT Filed: Dec. 2, 2014
(86) PCT No.: PCT/US2014/068225

§ 371 Date: May 25, 2016

Related U.S. Application Data
(60) Provisional application No. 61/910,871, filed on Dec. 2, 2013.

(51) Int. Cl. C12N 15/113

(52) U.S. Cl. CPC C12N 15/113; C12N 2310/11; C12N 2310/322; C12N 2310/3231

(57) ABSTRACT

The present invention provides compounds comprising oligonucleotides complementary to a CLN3 transcript. Certain such compounds are useful for hybridizing to a CLN3 transcript, including but not limited to a CLN3 transcript in a cell. In certain embodiments, such hybridization results in modulation of splicing of the CLN3 transcript. In certain embodiments, such compounds are used to treat one or more symptoms associated with Batten Disease.

### SEQUENCE LISTING

The present application is being filed along with a Sequence Listing in electronic format. The Sequence Listing is provided as a file entitled CORE0120WOSEQ_ST25.txt, created Nov. 25, 2014, which is 68 kb in size. The information in the electronic format of the sequence listing is incorporated herein by reference in its entirety.

### BACKGROUND

Juvenile neuronal ceroid lipofuscinosis (JNCL), also known as Batten Disease, is the most common of the NCL disorders, a group of childhood neurodegenerative diseases. JNCL occurs in approximately 1 in 25,000 births in the United States and Europe and has been reported in many other countries worldwide. Onset occurs between five and eight years of age and symptoms include progressive loss of motor function, seizures, vision loss, and loss of cognitive function, resulting in death before age 30. JNCL is an autosomal recessive disorder caused by mutations of the CLN3 gene. There are forty-nine known mutations of CLN3, but approximately 80% of JNCL cases result from a particular deletion of the CLN3 gene spanning exons 7 and 8 (CLN3Δ78). The CLN3Δ78 deletion causes a frameshift that results in a premature stop codon in exon 9. The truncated protein product of CLN3Δ78 is 33% of the length of the wild type. The function of the CLN3 protein is not well understood, but it is implicated in many important processes, for example, membrane trafficking, phospholipid distribution, and response to oxidative stress. Currently, there are no treatments for any of the NCL disorders, and patient options are limited to remedial management of symptoms (see Bennett and Rakheja, Dev. Disabil. Res. Rev. 2013, 17, 254-259).

Antisense compounds have been used to modulate target nucleic acids. Antisense compounds comprising a variety of chemical modifications and motifs have been reported. In certain instances, such compounds are useful as research tools, diagnostic reagents, and as therapeutic agents. In certain instances antisense compounds have been shown to modulate protein expression by binding to a target messenger RNA (mRNA) encoding the protein. In certain instances, such binding of an antisense compound to its target mRNA results in cleavage of the mRNA. Antisense compounds that modulate processing of a pre-mRNA have also been reported. Such antisense compounds alter splicing, interfere with polyadenlyation or prevent formation of the 5′-cap of a pre-mRNA.

Certain antisense compounds have been described previously. See for example U.S. Pat. No. 7,399,845 and published International Patent Application No. WO 2008/049085, which are hereby incorporated by reference herein in their entirety.

### SUMMARY

Many JNCL cases result from a particular deletion of the CLN3 gene spanning exons 7 and 8 (CLN3Δ78). The CLN3Δ78 deletion causes a frameshift that results in a premature stop codon in exon 9. The truncated protein product of CLN3Δ78 is 33% of the length of the wild type. In certain embodiments, the truncated CLN3Δ78 protein causes a variety of cellular defects, including lysosomal and transporter dysfunction. In certain embodiments, modified oligonucleotides targeted to CLN3 pre-mRNA are able to induce skipping of one or more exons and thereby prevent the frameshift that results in a premature stop codon in exon 9.

For example, in certain embodiments, modified oligonucleotides targeted to exon 6 CLN3 pre-mRNA may induce skipping on exon 6 and prevent recognition of the premature stop codon in exon 9, thereby producing a truncated CLN3 protein having restored functionality compared to the CLN3Δ78 isoform. In certain embodiments, modified oligonucleotides targeted to exon 9 CLN3 pre-mRNA may induce skipping on exon 9 and prevent recognition of the premature stop codon in exon 9, thereby producing a truncated CLN3 protein having restored functionality compared to the CLN3Δ78 isoform.

In certain embodiments, the present disclosure provides compounds comprising oligonucleotides. In certain embodiments, such oligonucleotides are complementary to a ceroid-lipofuscinosis, neuronal 3 (“CLN3”) transcript. In certain such embodiments, oligonucleotides are complementary to a target region of the CLN3 transcript comprising exon 6. In certain such embodiments, oligonucleotides are complementary to a target region of the CLN3 transcript comprising an intron adjacent to exon 6. In certain such embodiments, oligonucleotides are complementary to a target region of the CLN3 transcript comprising an intron adjacent to exon 6 and downstream of exon 6. In certain such embodiments, oligonucleotides are complementary to a target region of the CLN3 transcript comprising an intron adjacent to exon 6 and upstream of exon 6.

In certain such embodiments, oligonucleotides are complementary to a target region of the CLN3 transcript comprising exon 9. In certain such embodiments, oligonucleotides are complementary to a target region of the CLN3 transcript comprising an intron adjacent to exon 9. In certain such embodiments, oligonucleotides are complementary to a target region of the CLN3 transcript comprising an intron adjacent to exon 9 and downstream of exon 9. In certain such embodiments, oligonucleotides are complementary to a target region of the CLN3 transcript comprising an intron adjacent to exon 9 and upstream of exon 9.

In certain embodiments, oligonucleotides promote skipping of exon 6. In certain embodiments, oligonucleotides promote skipping of exon 6 of a CLN3Δ78 transcript. In certain embodiments, oligonucleotides promote skipping of exon 9. In certain embodiments, oligonucleotides promote skipping of exon 9 of a CLN3Δ78 transcript.

In certain embodiments, oligonucleotides promote skipping of exons 6, 7, and 8. In certain embodiments, oligonucleotides promote skipping of exons 7, 8, and 9. In certain embodiments, oligonucleotides promote skipping of exon 6. In certain embodiments, oligonucleotides promote skipping of exon 9.

In certain embodiments, including, but not limited to any of the above numbered embodiments, the CLN3 transcript is in a human. In certain embodiments, including, but not limited to any of the above numbered embodiments, the CLN3 transcript is in a mouse.

The present disclosure provides the following non-limiting numbered embodiments:

### Embodiment 1

A compound comprising a modified oligonucleotide consisting of 8 to 30 linked nucleosides and having a nucleobase sequence comprising a complementary region, wherein the complementary region comprises at least 8 contiguous nucleobases and is complementary to an equal-length portion of a target region of a CLN3 transcript.

### Embodiment 2

The compound of embodiment 1, wherein the target region of the CLN3 transcript comprises at least a portion of exon 6 of the CLN3 transcript.

### Embodiment 3

The compound of embodiment 1, wherein the target region of the CLN3 transcript comprises at least a portion of exon 9 of the CLN3 transcript.

### Embodiment 4

The compound of embodiment 1, wherein the target region of the CLN3 transcript comprises at least a portion of intron 5 of the CLN3 transcript.

### Embodiment 5

The compound of embodiment 1, wherein the target region of the CLN3 transcript comprises at least a portion of intron 6 of the CLN3 transcript.

### Embodiment 6

The compound of embodiment 1, wherein the target region of the CLN3 transcript comprises at least a portion of intron 9 of the CLN3 transcript.

### Embodiment 7

The compound of embodiment 1, wherein the target region of the CLN3 transcript comprises at least a portion of intron 10 of the CLN3 transcript.

### Embodiment 8

The compound of any of embodiments 1 to 7, wherein the complementary region of the modified oligonucleotide is 100% complementary to the target region.

### Embodiment 9

The compound of any of embodiments 1 to 8, wherein the complementary region of the modified oligonucleotide comprises at least 10 contiguous nucleobases.

### Embodiment 10

The compound of any of embodiments 1 to 8, wherein the complementary region of the modified oligonucleotide comprises at least 12 contiguous nucleobases.

### Embodiment 11

The compound of any of embodiments 1 to 8, wherein the complementary region of the modified oligonucleotide comprises at least 14 contiguous nucleobases.

### Embodiment 12

The compound of any of embodiments 1 to 8, wherein the complementary region of the modified oligonucleotide comprises at least 15 contiguous nucleobases.

### Embodiment 13

The compound of any of embodiments 1 to 8, wherein the complementary region of the modified oligonucleotide comprises at least 16 contiguous nucleobases.

### Embodiment 14

The compound of any of embodiments 1 to 8, wherein the complementary region of the modified oligonucleotide comprises at least 17 contiguous nucleobases.

### Embodiment 15

The compound of any of embodiments 1 to 8, wherein the complementary region of the modified oligonucleotide comprises at least 18 contiguous nucleobases.

### Embodiment 16

The compound of any of embodiments 1 to 8, wherein the complementary region of the modified oligonucleotide comprises at least 19 contiguous nucleobases.

### Embodiment 17

The compound of any of embodiments 1 to 8, wherein the complementary region of the modified oligonucleotide comprises at least 20 contiguous nucleobases.

### Embodiment 18

The compound of any of embodiments 1 to 17, wherein the nucleobase sequence of the oligonucleotide is at least 80% complementary to an equal-length region of the CLN3 transcript, as measured over the entire length of the oligonucleotide.

### Embodiment 19

The compound of any of embodiments 1 to 17, wherein the nucleobase sequence of the oligonucleotide is at least 90% complementary to an equal-length region of the CLN3 transcript, as measured over the entire length of the oligonucleotide.

### Embodiment 20

The compound of any of embodiments 1 to 17, wherein the nucleobase sequence of the oligonucleotide is 100% complementary to an equal-length region of the CLN3 transcript, as measured over the entire length of the oligonucleotide.

### Embodiment 21

The compound of any of embodiments 1-2 or 8 to 20, wherein the target region is within nucleobase 5053 and nucleobase 5070 of SEQ ID NO.: 2.

### Embodiment 22

The compound of any of embodiments 1-2 or 8 to 20, wherein the target region is within nucleobase 5082 and nucleobase 5119 of SEQ ID NO.: 2.

### Embodiment 23

The compound of any of embodiments 1-2 or 8 to 20, wherein the target region is within nucleobase 5082 and nucleobase 5099 of SEQ ID NO.: 2.

### Embodiment 24

The compound of any of embodiments 1-2 or 8 to 20, wherein the target region is within nucleobase 5086 and nucleobase 5103 of SEQ ID NO.: 2.

### Embodiment 25

The compound of any of embodiments 1-2 or 8 to 20, wherein the target region is within nucleobase 5090 and nucleobase 5107 of SEQ ID NO.: 2.

### Embodiment 26

The compound of any of embodiments 1-2 or 8 to 20, wherein the target region is within nucleobase 5094 and nucleobase 5111 of SEQ ID NO.: 2.

### Embodiment 27

The compound of any of embodiments 1-2 or 8 to 20, wherein the target region is within nucleobase 5098 and nucleobase 5115 of SEQ ID NO.: 2.

### Embodiment 28

The compound of any of embodiments 1-2 or 8 to 20, wherein the target region is within nucleobase 5102 and nucleobase 5119 of SEQ ID NO.: 2.

### Embodiment 29

The compound of any of embodiments 1-2, 5, or 8 to 20, wherein the target region is within nucleobase 5126 and nucleobase 5143 of SEQ ID NO.: 2.

### Embodiment 30

The compound of any of embodiments 1-2, 5 or 8 to 20, wherein the target region is within nucleobase 5134 and nucleobase 5155 of SEQ ID NO.: 2.

### Embodiment 31

The compound of any of embodiments 1-2, 5, or 8 to 20, wherein the target region is within nucleobase 5134 and nucleobase 5151 of SEQ ID NO.: 2.

### Embodiment 32

The compound of any of embodiments 1-2, 5 or 8 to 20, wherein the target region is within nucleobase 5138 and nucleobase 5155 of SEQ ID NO.: 2.

### Embodiment 33

The compound of any of embodiments 1, 3, or 8 to 20, wherein the target region is within nucleobase 7366 and nucleobase 7411 of SEQ ID NO.: 2.

### Embodiment 34

The compound of any of embodiments 1, 3, or 8 to 20, wherein the target region is within nucleobase 7366 and nucleobase 7383 of SEQ ID NO.: 2.

### Embodiment 35

The compound of any of embodiments 1, 3, or 8 to 20, wherein the target region is within nucleobase 7370 and nucleobase 7387 of SEQ ID NO.: 2.

### Embodiment 36

The compound of any of embodiments 1, 3, or 8 to 20, wherein the target region is within nucleobase 7371 and nucleobase 7388 of SEQ ID NO.: 2.

### Embodiment 37

The compound of any of embodiments 1, 3, or 8 to 20, wherein the target region is within nucleobase 7387 and nucleobase 7404 of SEQ ID NO.: 2.

### Embodiment 38

The compound of any of embodiments 1, 3, or 8 to 20, wherein the target region is within nucleobase 7394 and nucleobase 7411 of SEQ ID NO.: 2.

### Embodiment 39

The compound of any of embodiments 1, 3, 6, or 8 to 20, wherein the target region is within nucleobase 7454 and nucleobase 7471 of SEQ ID NO.: 2.

### Embodiment 40

The compound of any of embodiments 1, 3, 6, or 8 to 20, wherein the target region is within nucleobase 7462 and nucleobase 7483 of SEQ ID NO.: 2.

### Embodiment 41

The compound of any of embodiments 1, 3, 6, or 8 to 20, wherein the target region is within nucleobase 7462 and nucleobase 7479 of SEQ ID NO.: 2.

### Embodiment 42

The compound of any of embodiments 1, 3, 6, or 8 to 20, wherein the target region is within nucleobase 7466 and nucleobase 7483 of SEQ ID NO.: 2.

### Embodiment 43

The compound of any of embodiments 1-42, wherein the nucleobase sequence of the antisense oligonucleotide comprises any one of SEQ ID NOs: 3 to 60.

### Embodiment 44

The compound of any of embodiments 1-43, wherein the modified oligonucleotide comprises at least one modified nucleoside.

### Embodiment 45

The compound of embodiment 44, wherein at least one modified nucleoside comprises a modified sugar moiety.

### Embodiment 46

The compound of embodiment 45, wherein at least one modified sugar moiety is a 2′-substituted sugar moiety.

### Embodiment 47

The compound of embodiment 46, wherein the 2′-substitutent of at least one 2′-substituted sugar moiety is selected from among: 2′-OMe, 2′-F, and 2′-MOE.

### Embodiment 48

The compound of any of embodiments 44-47, wherein the 2′-substituent of at least one 2′-substituted sugar moiety is a 2′-MOE.

### Embodiment 49

The compound of any of embodiments 1-48, wherein at least one modified sugar moiety is a bicyclic sugar moiety.

### Embodiment 50

The compound of embodiment 49, wherein at least one bicyclic sugar moiety is LNA or cEt.

### Embodiment 51

The compound of any of embodiments 1-51, wherein at least one sugar moiety is a sugar surrogate.

### Embodiment 52

The compound of embodiment 51, wherein at least one sugar surrogate is a morpholino.

### Embodiment 53

The compound of embodiment 51, wherein at least one sugar surrogate is a modified morpholino.

### Embodiment 54

The compound of any of embodiment 1-53, wherein the modified oligonucleotide comprises at least 5 modified nucleosides, each independently comprising a modified sugar moiety.

### Embodiment 55

The compound of embodiment 54, wherein the modified oligonucleotide comprises at least 10 modified nucleosides, each independently comprising a modified sugar moiety.

### Embodiment 56

The compound of embodiment 54, wherein the modified oligonucleotide comprises at least 15 modified nucleosides, each independently comprising a modified sugar moiety.

### Embodiment 57

The compound of embodiment 54, wherein each nucleoside of the modified oligonucleotide is a modified nucleoside, each independently comprising a modified sugar moiety.

### Embodiment 58

The compound of any of embodiments 1-57, wherein the modified oligonucleotide comprises at least two modified nucleosides comprising modified sugar moieties that are the same as one another.

### Embodiment 59

The compound of any of embodiments 1-57, wherein the modified oligonucleotide comprises at least two modified nucleosides comprising modified sugar moieties that are different from one another.

### Embodiment 60

The compound of any of embodiments 1-59, wherein the modified oligonucleotide comprises a modified region of at least 5 contiguous modified nucleosides.

### Embodiment 61

The compound of any of embodiments 1 to 60, wherein the modified oligonucleotide comprises a modified region of at least 10 contiguous modified nucleosides.

### Embodiment 62

The compound of any of embodiments 1 to 61, wherein the modified oligonucleotide comprises a modified region of at least 15 contiguous modified nucleosides.

### Embodiment 63

The compound of any of embodiments 1 to 61, wherein the modified oligonucleotide comprises a modified region of at least 20 contiguous modified nucleosides.

### Embodiment 64

The compound of any of embodiments 58 to 63, wherein each modified nucleoside of the modified region has a modified sugar moiety independently selected from among: 2′-F, 2′-OMe, 2′-MOE, cEt, LNA, morpholino, and modified morpholino.

### Embodiment 65

The compound of any of embodiments 58 to 64, wherein the modified nucleosides of the modified region each comprise the same modification as one another.

### Embodiment 66

The compound of embodiment 65, wherein the modified nucleosides of the modified region each comprise the same 2′-substituted sugar moiety.

### Embodiment 67

The compound of embodiment 65, wherein the 2′-substituted sugar moiety of the modified nucleosides of the region of modified nucleosides is selected from 2′-F, 2′-OMe, and 2′-MOE.

### Embodiment 68

The compound of embodiment 67, wherein the 2′-substituted sugar moiety of the modified nucleosides of the region of modified nucleosides is 2′-MOE.

### Embodiment 69

The compound of embodiment 65, wherein the modified nucleosides of the region of modified nucleosides each comprise the same bicyclic sugar moiety.

### Embodiment 70

The compound of embodiment 69, wherein the bicyclic sugar moiety of the modified nucleosides of the region of modified nucleosides is selected from LNA and cEt.

### Embodiment 71

The compound of embodiment 65, wherein the modified nucleosides of the region of modified nucleosides each comprises a sugar surrogate.

### Embodiment 72

The compound of embodiment 71, wherein the sugar surrogate of the modified nucleosides of the region of modified nucleosides is a morpholino.

### Embodiment 73

The compound of embodiment 71, wherein the sugar surrogate of the modified nucleosides of the region of modified nucleosides is a modified morpholino.

### Embodiment 74

The compound of any of embodiments 1 to 73, wherein the modified nucleotide comprises no more than 4 contiguous naturally occurring nucleosides.

### Embodiment 75

The compound of any of embodiments 1 to 74, wherein each nucleoside of the modified oligonucleotide is a modified nucleoside.

### Embodiment 76

The compound of embodiment 75 wherein each modified nucleoside comprises a modified sugar moiety.

### Embodiment 77

The compound of embodiment 76, wherein the modified nucleosides of the modified oligonucleotide comprise the same modification as one another.

### Embodiment 78

The compound of embodiment 77, wherein the modified nucleosides of the modified oligonucleotide each comprise the same 2′-substituted sugar moiety.

### Embodiment 79

The compound of embodiment 78, wherein the 2′-substituted sugar moiety of the modified oligonucleotide is selected from 2′-F, 2′-OMe, and 2′-MOE.

### Embodiment 80

The compound of embodiment 78, wherein the 2′-substituted sugar moiety of the modified oligonucleotide is 2′-MOE.

### Embodiment 81

The compound of embodiment 77, wherein the modified nucleosides of the modified oligonucleotide each comprise the same bicyclic sugar moiety.

### Embodiment 82

The compound of embodiment 81, wherein the bicyclic sugar moiety of the modified oligonucleotide is selected from LNA and cEt.

### Embodiment 83

The compound of embodiment 77, wherein the modified nucleosides of the modified oligonucleotide each comprises a sugar surrogate.

### Embodiment 84

The compound of embodiment 83, wherein the sugar surrogate of the modified oligonucleotide is a morpholino.

### Embodiment 85

The compound of embodiment 83, wherein the sugar surrogate of the modified oligonucleotide is a modified morpholino.

### Embodiment 86

The compound of any of embodiments 1 to 85, wherein the modified oligonucleotide comprises at least one modified internucleoside linkage.

### Embodiment 87

The compound of embodiment 86, wherein each internucleoside linkage is a modified internucleoside linkage.

### Embodiment 88

The compound of embodiment 86 or 87, comprising at least one phosphorothioate internucleoside linkage.

### Embodiment 89

The compound of embodiment 86, wherein each internucleoside linkage is a modified internucleoside linkage and wherein each internucleoside linkage comprises the same modification.

### Embodiment 90

The compound of embodiment 89, wherein each internucleoside linkage is a phosphorothioate internucleoside linkage.

### Embodiment 91

The compound of any of embodiments 1 to 90, comprising at least one conjugate.

### Embodiment 92

The compound of any of embodiments 1 to 91 consisting of the modified oligonucleotide.

### Embodiment 93

The compound of any of embodiments 1 to 92, wherein the compound modulates splicing of the CLN3 transcript.

### Embodiment 94

The compound of any of embodiments 1 to 93, having a nucleobase sequence comprising any of the sequences as set forth in SEQ ID NOs. 3 to 60.

### Embodiment 95

The compound of any of embodiments 1, 2, 4 to 5, or 8 to 93, having a nucleobase sequence comprising any of the sequences as set forth in SEQ ID NOs. 8, 16, 17, 18, 19, 20, 21, 27, 29, or 30.

### Embodiment 96

The compound of any of embodiments 1, 3, or 6 to 93, having a nucleobase sequence comprising any of the sequences as set forth in SEQ ID NOs. 38, 39, 40, 41, 43, 55, 57, or 58.

### Embodiment 97

The compound of any of embodiments 1, 2, or 8 to 93, having a nucleobase sequence comprising SEQ ID NO. 20.

### Embodiment 98

A pharmaceutical composition comprising a compound according to any of embodiments 1-97 and a pharmaceutically acceptable carrier or diluent.

### Embodiment 99

The pharmaceutical composition of embodiment 98, wherein the pharmaceutically acceptable carrier or diluent is sterile saline.

### Embodiment 100

A method of modulating splicing of a CLN3 transcript in a cell comprising contacting the cell with a compound according to any of embodiments 1-99.

### Embodiment 101

The method of embodiment 100, wherein the cell is in vitro.

### Embodiment 102

The method of embodiment 100, wherein the cell is in an animal.

### Embodiment 103

The method of any of embodiments 100 to 102, wherein the amount of CLN3 mRNA without exon 6 is increased.

### Embodiment 104

The method of any of embodiments 100 to 102, wherein the amount of CLN3 mRNA without exon 9 is increased.

### Embodiment 105

The method of any of embodiments 100 to 102, wherein the amount of CLN3 mRNA with exon 10 is increased.

### Embodiment 106

The method of any of embodiments 100 to 105, wherein the CLN3 transcript is transcribed from a CLN3Δ78 gene.

### Embodiment 107

A method of modulating the expression of CLN3 in a cell, comprising contacting the cell with a compound according to any of embodiments 1-99.

### Embodiment 108

The method of embodiment 84, wherein the cell is in vitro.

### Embodiment 109

The method of embodiment 84, wherein the cell is in an animal.

### Embodiment 110

A method comprising administering the compound according to any of embodiments 1-97 or the pharmaceutical composition of embodiments 98 or 99 to an animal.

### Embodiment 111

The method of embodiment 108, wherein the administration is intracerebroventricular.

### Embodiment 112

The method of embodiment 108, wherein the administration is into the central nervous system.

### Embodiment 113

The method of any of embodiments 108-110, wherein the animal has one or more symptoms associated with Batten Disease.

### Embodiment 114

The method of any of embodiments 108-110, wherein the administration results in amelioration of at least one symptom of Batten Disease.

### Embodiment 115

The method of any of embodiments 108 to 112, wherein the animal is a mouse.

### Embodiment 116

The method of any of embodiments 108 to 112, wherein the animal is a human.

### Embodiment 117

A method of preventing or slowing one or more symptoms Batten Disease, comprising administering the compound according to any of embodiments 1-97 or the pharmaceutical composition of embodiments 98 or 99 to an animal in need thereof.

### Embodiment 118

Use of the compound according to any of embodiments 1-97 or the pharmaceutical composition of embodiments 98 or 99 for the preparation of a medicament for use in the treatment of Batten Disease.

### Embodiment 119

Use of the compound according to any of embodiments 1-97 or the pharmaceutical composition of embodiments 98 or 99 for the preparation of a medicament for use in the amelioration of one or more symptoms Batten Disease.

### Embodiment 120

A compound comprising a modified oligonucleotide consisting of 8 to 30 linked nucleosides and having a nucleobase sequence comprising a complementary region, wherein the complementary region comprises at least 8 contiguous nucleobases and is complementary to an equal-length portion of a target region of a CLN3 transcript.

### Embodiment 121

The compound of embodiment 120, wherein the CLN3 transcript comprises the nucleobase sequence of SEQ ID NO. 1.

### Embodiment 122

The compound of embodiment 120 or 121, wherein the complementary region of the modified oligonucleotide is 100% complementary to the target region.

### Embodiment 123

The compound of any of embodiments 120 to 122, wherein the complementary region of the modified oligonucleotide comprises at least 10 contiguous nucleobases.

### Embodiment 124

The compound of any of embodiments 120 to 122, wherein the complementary region of the modified oligonucleotide comprises at least 12 contiguous nucleobases.

### Embodiment 125

The compound of any of embodiments 120 to 122, wherein the complementary region of the modified oligonucleotide comprises at least 14 contiguous nucleobases.

### Embodiment 126

The compound of any of embodiments 120 to 122, wherein the complementary region of the modified oligonucleotide comprises at least 15 contiguous nucleobases.

### Embodiment 127

The compound of any of embodiments 120 to 122, wherein the complementary region of the modified oligonucleotide comprises at least 16 contiguous nucleobases.

### Embodiment 128

The compound of any of embodiments 120 to 122, wherein the complementary region of the modified oligonucleotide comprises at least 17 contiguous nucleobases.

### Embodiment 129

The compound of any of embodiments 120 to 122, wherein the complementary region of the modified oligonucleotide comprises at least 18 contiguous nucleobases.

### Embodiment 130

The compound of any of embodiments 120 to 129, wherein the nucleobase sequence of the modified oligonucleotide is at least 80% complementary to an equal-length region of the CLN3 transcript, as measured over the entire length of the oligonucleotide.

### Embodiment 131

The compound of any of embodiments 120 to 129, wherein the nucleobase sequence of the modified oligonucleotide is at least 90% complementary to an equal-length region of the CLN3 transcript, as measured over the entire length of the oligonucleotide.

### Embodiment 132

The compound of any of embodiments 120 to 129, wherein the nucleobase sequence of the modified oligonucleotide is 100% complementary to an equal-length region of the CLN3 transcript, as measured over the entire length of the oligonucleotide.

### Embodiment 133

The compound of any of embodiments 120-132, wherein the target region is within nucleobase 5704-5721, 5709-5726, 5714-5731, 5734-5751, 5764-5781, 5769-5786, 5774-5791, 5779-5796, 5784-5801, 5789-5806, 5794-5811, 5799-5816, 5804-5821, 5809-5826, 5814-5831, 5819-5836, 5824-5841, 5829-5846, 5834-5851, 5839-5856, 5844-5861, 5849-5866, 5854-5871, 5859-5876, 5879-5896, 5884-5901, or 5889-5906 of SEQ ID NO.: 1.

### Embodiment 134

The compound of any of embodiments 120-133, wherein the target region is within nucleobase 5784-5801 of SEQ ID NO.: 1.

### Embodiment 135

The compound of any of embodiments 120-132, wherein the target region is within nucleobase 5804-5821 of SEQ ID NO.: 1.

### Embodiment 136

The compound of any of embodiments 120-132, wherein the target region is within nucleobase 9122-9139, 9127-9144, 9132-9149, 9137-9154, 9142-9159, 9147-9164, 9152-9169, 9157-9174, 9162-9179, 9167-9184, 9172-9189, 9177-9194, 9182-9199, 9187-9204, 9192-9209, 9197-9214, 9202-9219, 9207-9224, 9212-9229, 9217-9234, 9222-9239, 9227-9244, 9232-9249, 9237-9254, 9242-9259, 9262-9279, 9282-9299, 9287-9304, 9292-9309, 9297-9314, 9302-9319, 9307-9324, 9312-9329, 9317-9334, 9322-9339, 9327-9344, 9332-9349, or 9337-9354 of SEQ ID NO.: 1.

### Embodiment 137

The compound of any of embodiments 120-132, wherein the modified oligonucleotide has a nucleobase sequence comprising any of the sequences as set forth in SEQ ID NOs: 63, 64, 65, 66, 67, 68, 69, 70, 71, 72, 73, 74, 75, 76, 77, 78, 79, 80, 81, 82, 83, 84, 85, 86, 87, 88, 89, or 90.

### Embodiment 138

The compound of any of embodiments 120-132, wherein the modified oligonucleotide has a nucleobase sequence consisting of the nucleobase sequence of any one of SEQ ID NOs: 63, 64, 65, 66, 67, 68, 69, 70, 71, 72, 73, 74, 75, 76, 77, 78, 79, 80, 81, 82, 83, 84, 85, 86, 87, 88, 89, or 90.

### Embodiment 139

The compound of any of embodiments 120-132, wherein the modified oligonucleotide has a nucleobase sequence comprising the nucleobase sequence of SEQ ID NO. 72.

### Embodiment 140

The compound of embodiment 138, wherein the modified oligonucleotide has a nucleobase sequence consisting of the nucleobase sequence of SEQ ID NO. 72.

### Embodiment 141

The compound of any of embodiments 120-132, wherein the modified oligonucleotide has a nucleobase sequence comprising the nucleobase sequence of SEQ ID NO. 73.

### Embodiment 142

The compound of embodiment 138, wherein the modified oligonucleotide has a nucleobase sequence consisting of the nucleobase sequence of SEQ ID NO. 73.

### Embodiment 143

The compound of any of embodiments 120-132, wherein the modified oligonucleotide has a nucleobase sequence comprising any of the sequences as set forth in SEQ ID NOs: 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, or 127.

### Embodiment 144

The compound of any of embodiments 120-132, wherein the modified oligonucleotide has a nucleobase sequence consisting of the nucleobase sequence of any one of SEQ ID NOs: 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, or 127.

### Embodiment 145

The compound of any of embodiments 120-132, wherein the modified oligonucleotide has a nucleobase sequence comprising the nucleobase sequence of SEQ ID NO. 112.

### Embodiment 146

The compound of embodiment 144, wherein the modified oligonucleotide has a nucleobase sequence consisting of the nucleobase sequence of SEQ ID NO. 112.

### Embodiment 147

The compound of any of embodiments 120-146, wherein the modified oligonucleotide comprises at least one modified nucleoside.

### Embodiment 148

The compound of any of embodiments 120-146, wherein each nucleoside of the modified oligonucleotide is a modified nucleoside selected from among: 2′-OMe, 2′-F, and 2′-MOE or a sugar surrogate.

### Embodiment 149

The compound of embodiment 148, wherein the modified nucleoside is 2′-MOE.

### Embodiment 150

The compound of embodiment 148, wherein the modified nucleoside is a morpholino.

### Embodiment 151

The compound of embodiment 147, wherein at least one modified nucleoside comprises a modified sugar moiety.

### Embodiment 152

The compound of embodiment 151, wherein at least one modified sugar moiety is a 2′-substituted sugar moiety.

### Embodiment 153

The compound of embodiment 152, wherein the 2′-substitutent of at least one 2′-substituted sugar moiety is selected from among: 2′-OMe, 2′-F, and 2′-MOE.

### Embodiment 154

The compound of any of embodiments 152-153, wherein the 2′-substituent of at least one 2′-substituted sugar moiety is a 2′-MOE.

### Embodiment 155

The compound of any of embodiments 120-154, wherein at least one modified sugar moiety is a bicyclic sugar moiety.

### Embodiment 156

The compound of embodiment 155, wherein at least one bicyclic sugar moiety is LNA or cEt.

### Embodiment 157

The compound of any of embodiments 120-1563, wherein at least one sugar moiety is a sugar surrogate.

### Embodiment 158

The compound of embodiment 157, wherein at least one sugar surrogate is a morpholino.

### Embodiment 159

The compound of embodiment 158, wherein at least one sugar surrogate is a modified morpholino.

### Embodiment 160

The compound of any of embodiments 120-159, wherein the modified oligonucleotide comprises at least 5 modified nucleosides, each independently comprising a modified sugar moiety.

### Embodiment 161

The compound of any of embodiments 120-159, wherein the modified oligonucleotide comprises at least 10 modified nucleosides, each independently comprising a modified sugar moiety.

### Embodiment 162

The compound of any of embodiments 120-159, wherein the modified oligonucleotide comprises at least 15 modified nucleosides, each independently comprising a modified sugar moiety.

### Embodiment 163

The compound of any of embodiments 120-159, wherein each nucleoside of the modified oligonucleotide is a modified nucleoside, each independently comprising a modified sugar moiety.

### Embodiment 164

The compound of any of embodiments 120-163, wherein the modified oligonucleotide comprises at least two modified nucleosides comprising modified sugar moieties that are the same as one another.

### Embodiment 165

The compound of any of embodiments 120-163, wherein the modified oligonucleotide comprises at least two modified nucleosides comprising modified sugar moieties that are different from one another.

### Embodiment 166

The compound of any of embodiments 120-165, wherein the modified oligonucleotide comprises a modified region of at least 5 contiguous modified nucleosides.

### Embodiment 167

The compound of any of embodiments 120-165, wherein the modified oligonucleotide comprises a modified region of at least 10 contiguous modified nucleosides.

### Embodiment 168

The compound of any of embodiments 120-165, wherein the modified oligonucleotide comprises a modified region of at least 15 contiguous modified nucleosides.

### Embodiment 169

The compound of any of embodiments 120-165, wherein the modified oligonucleotide comprises a modified region of at least 16 contiguous modified nucleosides.

### Embodiment 170

The compound of any of embodiments 120-165, wherein the modified oligonucleotide comprises a modified region of at least 17 contiguous modified nucleosides.

### Embodiment 171

The compound of any of embodiments 120-165, wherein the modified oligonucleotide comprises a modified region of at least 18 contiguous modified nucleosides.

### Embodiment 172

The compound of any of embodiments 120-165, wherein the modified oligonucleotide comprises a modified region of at least 20 contiguous modified nucleosides.

### Embodiment 173

The compound of any of embodiments 166 to 172, wherein each modified nucleoside of the modified region has a modified sugar moiety independently selected from among: 2′-F, 2′-OMe, 2′-MOE, cEt, LNA, morpholino, and modified morpholino.

### Embodiment 174

The compound of any of embodiments 166 to 173, wherein the modified nucleosides of the modified region each comprise the same modification as one another.

### Embodiment 175

The compound of embodiment 174, wherein the modified nucleosides of the modified region each comprise the same 2′-substituted sugar moiety.

### Embodiment 176

The compound of embodiment 174, wherein the 2′-substituted sugar moiety of the modified nucleosides of the region of modified nucleosides is selected from 2′-F, 2′-OMe, and 2′-MOE.

### Embodiment 177

The compound of embodiment 174, wherein the 2′-substituted sugar moiety of the modified nucleosides of the region of modified nucleosides is 2′-MOE.

### Embodiment 178

The compound of embodiment 174, wherein the modified nucleosides of the region of modified nucleosides each comprise the same bicyclic sugar moiety.

### Embodiment 179

The compound of embodiment 178, wherein the bicyclic sugar moiety of the modified nucleosides of the region of modified nucleosides is selected from LNA and cEt.

### Embodiment 180

The compound of embodiment 174, wherein the modified nucleosides of the region of modified nucleosides each comprises a sugar surrogate.

### Embodiment 181

The compound of embodiment 180, wherein the sugar surrogate of the modified nucleosides of the region of modified nucleosides is a morpholino.

### Embodiment 182

The compound of embodiment 180, wherein the sugar surrogate of the modified nucleosides of the region of modified nucleosides is a modified morpholino.

### Embodiment 183

The compound of any of embodiments 120 to 182, wherein the modified nucleotide comprises no more than 4 contiguous naturally occurring nucleosides.

### Embodiment 184

The compound of any of embodiments 120 to 183, wherein each nucleoside of the modified oligonucleotide is a modified nucleoside.

### Embodiment 185

The compound of embodiment 184 wherein each modified nucleoside comprises a modified sugar moiety.

### Embodiment 186

The compound of embodiment 185, wherein the modified nucleosides of the modified oligonucleotide comprise the same modification as one another.

### Embodiment 187

The compound of embodiment 186, wherein the modified nucleosides of the modified oligonucleotide each comprise the same 2′-substituted sugar moiety.

### Embodiment 188

The compound of embodiment 187, wherein the 2′-substituted sugar moiety of the modified oligonucleotide is selected from 2′-F, 2′-OMe, and 2′-MOE.

### Embodiment 189

The compound of embodiment 187, wherein the 2′-substituted sugar moiety of the modified oligonucleotide is 2′-MOE.

### Embodiment 190

The compound of embodiment 186, wherein the modified nucleosides of the modified oligonucleotide each comprise the same bicyclic sugar moiety.

### Embodiment 191

The compound of embodiment 190, wherein the bicyclic sugar moiety of the modified oligonucleotide is selected from LNA and cEt.

### Embodiment 192

The compound of embodiment 186, wherein the modified nucleosides of the modified oligonucleotide each comprises a sugar surrogate.

### Embodiment 193

The compound of embodiment 192, wherein the sugar surrogate of the modified oligonucleotide is a morpholino.

### Embodiment 194

The compound of embodiment 192, wherein the sugar surrogate of the modified oligonucleotide is a modified morpholino.

### Embodiment 195

The compound of any of embodiments 120 to 194, wherein the modified oligonucleotide comprises at least one modified internucleoside linkage.

### Embodiment 196

The compound of embodiment 195, wherein each internucleoside linkage is a modified internucleoside linkage.

### Embodiment 197

The compound of embodiment 195 or 196, comprising at least one phosphorothioate internucleoside linkage.

### Embodiment 198

The compound of embodiment 196, wherein each internucleoside linkage is a modified internucleoside linkage and wherein each internucleoside linkage comprises the same modification.

### Embodiment 199

The compound of embodiment 198, wherein each internucleoside linkage is a phosphorothioate internucleoside linkage.

### Embodiment 200

The compound of any of embodiments 120 to 200, comprising at least one conjugate.

### Embodiment 201

The compound of any of embodiments 120 to 200, consisting of the modified oligonucleotide.

### Embodiment 202

The compound of any of embodiments 120 to 201, wherein the compound modulates splicing of the CLN3 transcript.

### Embodiment 203

A pharmaceutical composition comprising a compound according to any of embodiments 120-202 and a pharmaceutically acceptable carrier or diluent.

### Embodiment 204

The pharmaceutical composition of embodiment 202, wherein the pharmaceutically acceptable carrier or diluent is sterile saline.

### Embodiment 205

A method of modulating splicing of a CLN3 transcript in a cell comprising contacting the cell with a compound according to any of embodiments 120-204.

### Embodiment 206

The method of embodiment 205, wherein the cell is in vitro.

### Embodiment 207

The method of embodiment 205, wherein the cell is in an animal.

### Embodiment 208

The method of any of embodiments 205 to 207, wherein the amount of CLN3 mRNA without exon 6 is increased.

### Embodiment 209

The method of any of embodiments 205 to 207, wherein the amount of CLN3 mRNA without exon 9 is increased.

### Embodiment 210

The method of any of embodiments 205 to 209, wherein the amount of CLN3 mRNA with exon 10 is increased.

### Embodiment 211

The method of any of embodiments 205 to 210, wherein the CLN3 transcript is transcribed from a CLN3Δ78 gene.

### Embodiment 212

A method of modulating the expression of CLN3 in a cell, comprising contacting the cell with a compound according to any of embodiments 120-204.

### Embodiment 213

The method of embodiment 212, wherein the cell is in vitro.

### Embodiment 214

The method of embodiment 212, wherein the cell is in an animal.

### Embodiment 215

A method comprising administering the compound of any of embodiments 120-204 to an animal.

### Embodiment 216

The method of embodiment 215, wherein the administration is intracerebroventricular.

### Embodiment 217

The method of embodiment 215, wherein the administration is into the central nervous system.

### Embodiment 218

The method of any of embodiments 215 to 217, wherein the animal has one or more symptoms associated with Batten Disease.

### Embodiment 219

The method of any of embodiments 215 to 217, wherein the administration results in amelioration of at least one symptom of Batten Disease.

### Embodiment 220

The method of any of embodiments 215 to 219, wherein the animal is a mouse.

### Embodiment 221

The method of any of embodiments 215 to 219, wherein the animal is a human.

### Embodiment 222

A method of preventing or slowing one or more symptoms Batten Disease, comprising administering the compound according to any of embodiments 120-204 to an animal in need thereof.

### Embodiment 223

The method of embodiment 222, wherein the animal is a human.

### Embodiment 224

Use of the compound according to any of embodiments 120-204 for the preparation of a medicament for use in the treatment of Batten Disease.

### Embodiment 225

Use of the compound according to any of embodiments 120-204 for the preparation of a medicament for use in the amelioration of one or more symptoms Batten Disease.

### BRIEF DESCRIPTION OF THE FIGURES

FIG. 1 is a schematic of the human WT CLN3 and mutant CLN3Δ78 RNA and proteins. The splice products, CLN3Δ678 and CLN3Δ789, are depicted as well. Exons are depicted as boxes, introns are depicted as lines. In certain embodiments, ASOs are used to alter the splicing of CLN3Δ78 pre-mRNA.

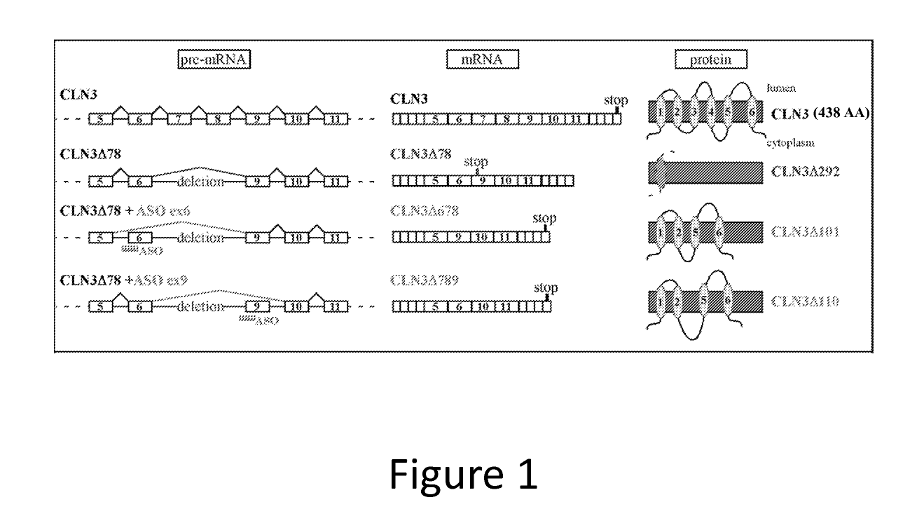

FIG. 2 shows splice products produced following treatment of WT, heterozygous, and homozygous mutant CLN3Δ78 fibroblasts with an ASO targeted to exon 6 of CLN3 and analysis by RT-PCR.

FIG. 3 shows splice products produced following treatment of homozygous mutant CLN3Δ78 fibroblasts with varying concentrations of an ASO targeted to exon 6 of CLN3 and analysis by RT-PCR.

FIG. 4 shows a western blot illustrating protein products produced following treatment of WT, heterozygous, and homozygous mutant CLN3Δ78 fibroblasts with an ASO targeted to exon 6 and a control ASO.

FIG. 5 shows the RT-PCR results of a screen of ASOs targeted to exon 6 of mouse CLN3.

FIG. 6 shows the RT-PCR results of a screen of ASOs targeted to exon 9 of mouse CLN3.

FIG. 7 shows splice products produced following treatment of homozygous mutant CLN3Δ78 Batten mice with an ASO targeted to exon 6 of CLN3 (616709) or a control ASO and analysis by RT-PCR.

FIG. 8 shows the RT-PCR results of a screen of ASOs targeted to exon 6 of human CLN3.

FIG. 9 shows the RT-PCR results of a screen of ASOs targeted to exon 9 of human CLN3.

FIG. 10 shows splice products produced following treatment of homozygous mutant CLN3Δ78 fibroblasts with ASOs targeted to exon 6 or exon 9 of human CLN3.

### DETAILED DESCRIPTION

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “chemical modification” means a chemical difference in a compound when compared to a naturally occurring counterpart. In reference to an oligonucleotide, chemical modification does not include differences only in nucleobase sequence. Chemical modifications of oligonucleotides include nucleoside modifications (including sugar moiety modifications and nucleobase modifications) and internucleoside linkage modifications.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA.

As used herein, “sugar moiety” means a naturally occurring sugar moiety or a modified sugar moiety of a nucleoside.

As used herein, “modified sugar moiety” means a substituted sugar moiety, a bicyclic or tricyclic sugar moiety, or a sugar surrogate.

As used herein, “substituted sugar moiety” means a furanosyl comprising at least one substituent group that differs from that of a naturally occurring sugar moiety. Substituted sugar moieties include, but are not limited to furanosyls comprising substituents at the 2′-position, the 3′-position, the 5′-position and/or the 4′-position.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

As used herein, “MOE” means —OCH₂CH₂OCH₃.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

As used herein the term “sugar surrogate” means a structure that does not comprise a furanosyl and that is capable of replacing the naturally occurring sugar moiety of a nucleoside, such that the resulting nucleoside is capable of (1) incorporation into an oligonucleotide and (2) hybridization to a complementary nucleoside. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholino, modified morpholinos, cyclohexenyls and cyclohexitols.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein, “heterocyclic base” or “heterocyclic nucleobase” means a nucleobase comprising a heterocyclic structure.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “bicyclic nucleoside” or “BNA” means a nucleoside comprising a bicyclic sugar moiety.

As used herein, “constrained ethyl nucleoside” or “cEt” means a nucleoside comprising a bicyclic sugar moiety comprising a 4′-CH(CH₃)—O-2′bridge.

As used herein, “locked nucleic acid nucleoside” or “LNA” means a nucleoside comprising a bicyclic sugar moiety comprising a 4′-CH₂—O-2′bridge.

As used herein, “2′-substituted nucleoside” means a nucleoside comprising a substituent at the 2′-position other than H or OH. Unless otherwise indicated, a 2′-substituted nucleoside is not a bicyclic nucleoside.

As used herein, “2′-deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein, “modified oligonucleotide” means an oligonucleotide comprising at least one modified nucleoside and/or at least one modified internucleoside linkage.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage” means any internucleoside linkage other than a naturally occurring internucleoside linkage.

As used herein, “oligomeric compound” means a polymeric structure comprising two or more sub-structures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide.

As used herein, “terminal group” means one or more atom attached to either, or both, the 3′ end or the 5′ end of an oligonucleotide. In certain embodiments a terminal group is a conjugate group. In certain embodiments, a terminal group comprises one or more terminal group nucleosides.

As used herein, “conjugate” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

As used herein, “conjugate linking group” means any atom or group of atoms used to attach a conjugate to an oligonucleotide or oligomeric compound.

As used herein, “antisense compound” means a compound comprising or consisting of an oligonucleotide at least a portion of which is complementary to a target nucleic acid to which it is capable of hybridizing, resulting in at least one antisense activity.

As used herein, “antisense activity” means any detectable and/or measurable change attributable to the hybridization of an antisense compound to its target nucleic acid.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measureable activity” means a statistically significant activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “target nucleic acid” means a nucleic acid molecule to which an antisense compound hybridizes.

As used herein, “mRNA” means an RNA molecule that encodes a protein.

As used herein, “pre-mRNA” means an RNA transcript that has not been fully processed into mRNA. Pre-RNA includes one or more intron.

As used herein, “transcript” means an RNA molecule transcribed from DNA. Transcripts include, but are not limited to mRNA, pre-mRNA, and partially processed RNA.

As used herein, “CLN3” means ceroid-lipofuscinosis, neuronal 3.

As used herein, “CLN3 transcript” means a transcript transcribed from a CLN3 gene. In certain embodiments, a CLN3 transcript comprises SEQ ID NO: 1: the complement of GENBANK accession number NT_010393.16 truncated from nucleotides 28427600 to 28444620. In certain embodiments, a CLN3 transcript comprises SEQ ID NO: 2: the complement of GENBANK accession number NT_039433.8, truncated from nucleotides 44319075 to 44333955.

As used herein, “CLN3 gene” means a gene that encodes a ceroid-lipofuscinosis, neuronal 3 protein and any ceroid-lipofuscinosis, neuronal 3 protein isoforms. In certain embodiments, a CLN3 gene is represented by GENBANK accession number NT_010393.16 truncated from nucleotides 28427600 to 28444620, or a variant thereof. In certain embodiments, a CLN3 gene is at least 95% identical to GENBANK accession number NT_010393.16 truncated from nucleotides 28427600 to 28444620. In certain embodiments, a CLN3 gene is at least 90% identical to GENBANK accession number NT_010393.16 truncated from nucleotides 28427600 to 28444620. In certain embodiments, a CLN3 gene is represented by GENBANK accession number NT_039433.8, truncated from nucleotides 44319075 to 44333955, or a variant thereof. In certain embodiments, a CLN3 gene is at least 95% identical to GENBANK accession number NT_039433.8, truncated from nucleotides 44319075 to 44333955. In certain embodiments, a CLN3 gene is at least 90% identical to GENBANK accession number NT_039433.8, truncated from nucleotides 44319075 to 44333955. In certain embodiments, a CLN3 gene encodes a wild-type CLN3 protein. In certain embodiments, a CLN3 gene encodes a CLN3Δ78 protein.

As used herein, “CLN3Δ78” means a CLN3 gene having a deletion spanning all or part of exons 7 and 8. In certain embodiments, the CLN3Δ78 deletion causes a frameshift that result in a premature stop codon in exon 9. In certain embodiments, the truncated protein product of CLN3Δ78 is 33% of the length of the wild type.

As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

As used herein, “motif” means a pattern of chemical modifications in an oligomeric compound or a region thereof. Motifs may be defined by modifications at certain nucleosides and/or at certain linking groups of an oligomeric compound.

As used herein, “nucleoside motif” means a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligomeric compound or a region thereof.

As used herein, “linkage motif” means a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

As used herein, “nucleobase modification motif” means a pattern of modifications to nucleobases along an oligonucleotide. Unless otherwise indicated, a nucleobase modification motif is independent of the nucleobase sequence.

As used herein, “sequence motif” means a pattern of nucleobases arranged along an oligonucleotide or portion thereof. Unless otherwise indicated, a sequence motif is independent of chemical modifications and thus may have any combination of chemical modifications, including no chemical modifications.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

As used herein, “the same type of modifications” refers to modifications that are the same as one another, including absence of modifications. Thus, for example, two unmodified DNA nucleoside have “the same type of modification,” even though the DNA nucleoside is unmodified. Such nucleosides having the same type modification may comprise different nucleobases.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present invention have substituents at one or at more than one position of the parent compound. Substituents may also be further substituted with other substituent groups and may be attached directly or via a linking group such as an alkyl or hydrocarbyl group to a parent compound.

Likewise, as used herein, “substituent” in reference to a chemical functional group means an atom or group of atoms differs from the atom or a group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group). Unless otherwise indicated, groups amenable for use as substituents include without limitation, halogen, hydroxyl, alkyl, alkenyl, alkynyl, acyl (—C(O)Rₐₐ), carboxyl (—C(O)O—Rₐₐ), aliphatic groups, alicyclic groups, alkoxy, substituted oxy (—O—Rₐₐ), aryl, aralkyl, heterocyclic radical, heteroaryl, heteroarylalkyl, amino (—N(R_{bb})(R_{cc})), imino(═NR_{bb}), amido (—C(O)N(R_{bb})(R_{cc}) or —N(R_{bb})C(O)Rₐₐ), azido (—N₃), nitro (—NO₂), cyano (—CN), carbamido (—OC(O)N(R_{bb})(R_{cc}) or —N(R_{bb})C(O)ORₐₐ), ureido (—N(R_{bb})C(O)N(R_{bb})(R_{cc})), thioureido (—N(R_{bb})C(S)N(R_{bb})—(R_{cc})), guanidinyl (—N(R_{bb})C(═NR_{bb})N(R_{bb})(R_{cc})), amidinyl (—C(═NR_{bb})N(R_{bb})(R_{cc}) or —N(R_{bb})C(═NR_{bb})(Rₐₐ)), thiol (—SR_{bb}), sulfinyl (—S(O)R_{bb}), sulfonyl (—S(O)₂R_{bb}) and sulfonamidyl (—S(O)₂N(R_{bb})(R_{cc}) or —N(R_{bb})S—(O)₂R_{bb}). Wherein each Rₐₐ, R_{bb} and R_{cc} is, independently, H, an optionally linked chemical functional group or a further substituent group with a preferred list including without limitation, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

As used herein, “alkyl,” as used herein, means a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred.

As used herein, “alkenyl,” means a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, “alkynyl,” means a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, “acyl,” means a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

As used herein, “alicyclic” means a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, “aliphatic” means a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines. Aliphatic groups as used herein may optionally include further substituent groups.

As used herein, “alkoxy” means a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, “aminoalkyl” means an amino substituted C₁-C₁₂ alkyl radical. The alkyl portion of the radical forms a covalent bond with a parent molecule. The amino group can be located at any position and the aminoalkyl group can be substituted with a further substituent group at the alkyl and/or amino portions.

As used herein, “aralkyl” and “arylalkyl” mean an aromatic group that is covalently linked to a C₁-C₁₂ alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

As used herein, “aryl” and “aromatic” mean a mono- or polycyclic carbocyclic ring system radicals having one or more aromatic rings. Examples of aryl groups include without limitation, phenyl, naphthyl, tetrahydronaphthyl, indanyl, indenyl and the like. Preferred aryl ring systems have from about 5 to about 20 carbon atoms in one or more rings. Aryl groups as used herein may optionally include further substituent groups.

As used herein, “halo” and “halogen,” mean an atom selected from fluorine, chlorine, bromine and iodine.

As used herein, “heteroaryl,” and “heteroaromatic,” mean a radical comprising a mono- or polycyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

### Oligomeric Compounds

In certain embodiments, the present invention provides oligomeric compounds. In certain embodiments, such oligomeric compounds comprise oligonucleotides optionally comprising one or more conjugate and/or terminal groups. In certain embodiments, an oligomeric compound consists of an oligonucleotide. In certain embodiments, oligonucleotides comprise one or more chemical modifications. Such chemical modifications include modifications one or more nucleoside (including modifications to the sugar moiety and/or the nucleobase) and/or modifications to one or more internucleoside linkage.

Certain Sugar Moieties

In certain embodiments, oligomeric compounds of the invention comprise one or more modified nucleosides comprising a modified sugar moiety. Such oligomeric compounds comprising one or more sugar-modified nucleosides may have desirable properties, such as enhanced nuclease stability or increased binding affinity with a target nucleic acid relative to oligomeric compounds comprising only nucleosides comprising naturally occurring sugar moieties. In certain embodiments, modified sugar moieties are substituted sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of substituted sugar moieties.

In certain embodiments, modified sugar moieties are substituted sugar moieties comprising one or more substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; O—C₁-C₁₀ alkoxy; O—C₁-C₁₀ substituted alkoxy, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In certain embodiments, substituted sugars comprise more than one non-bridging sugar substituent, for example, 2′-F-5′-methyl sugar moieties (see, e.g., PCT International Application WO 2008/101157, for additional 5′, 2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, O—C₁-C₁₀ alkoxy; O—C₁-C₁₀ substituted alkoxy, SH, CN, OCN, CF₃, OCF₃, O-alkyl, S-alkyl, N(Rₘ)-alkyl; O-alkenyl, S-alkenyl, or N(Rₘ)-alkenyl; O-alkynyl, S-alkynyl, N(Rₘ)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)₃-2′, 4′-(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845, issued on Jul. 15, 2008); 4′-C(CH₃)(CH₃)—O-2′ and analogs thereof, (see, e.g., WO2009/006478, published Jan. 8, 2009); 4′-CH₂—N(OCH₃)-2′ and analogs thereof (see, e.g., WO2008/150729, published Dec. 11, 2008); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see, e.g., Chattopadhyaya, et al., J. Org. Chem., 2009, 74, 118-134); and 4′-CH₂—C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH2-N(R)-2′) BNA, (I) methyl carbocyclic (4′-CH₂—CH(CH₃)-2′) BNA, and (J) propylene carbocyclic (4′-(CH₂)₃-2′) BNA as depicted below.

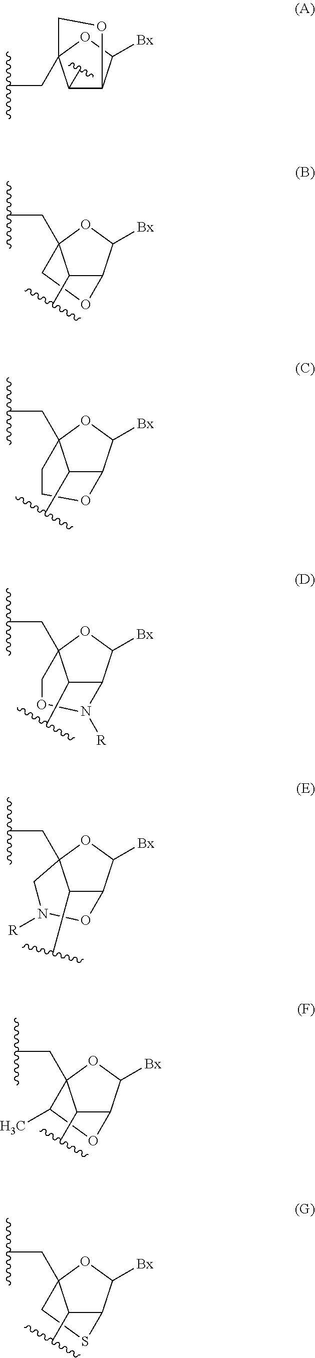

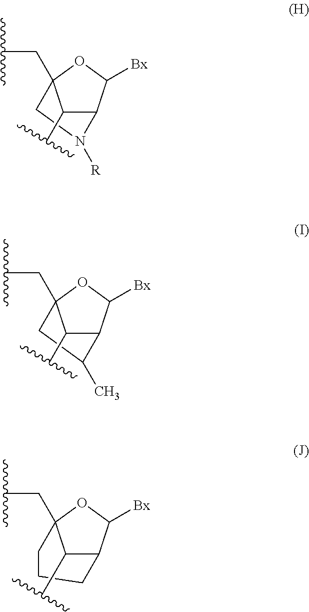

wherein Bx is a nucleobase moiety and R is, independently, H, a protecting group, or C₁-C₁₂ alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars). (see, PCT International Application WO 2007/134181, published on Nov. 22, 2007, wherein LNA is substituted with, for example, a 5′-methyl or a 5′-vinyl group).

In certain embodiments, modified sugar moieties are sugar surrogates. In certain such embodiments, the oxygen atom of the naturally occurring sugar is substituted, e.g., with a sulfur, carbon or nitrogen atom. In certain such embodiments, such modified sugar moiety also comprises bridging and/or non-bridging substituents as described above. For example, certain sugar surrogates comprise a 4′-sulfur atom and a substitution at the 2′-position (see, e.g., published U.S. Patent Application US2005/0130923, published on Jun. 16, 2005) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In certain embodiments, sugar surrogates comprise rings having other than 5-atoms. For example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), mannitol nucleic acid (MNA) (see Leumann, C J. Bioorg. & Med. Chem. (2002) 10:841-854), fluoro HNA (F-HNA), and those compounds having Formula VII:

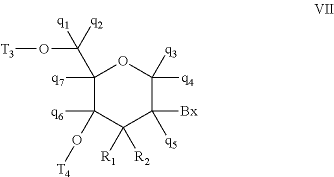

wherein independently for each of said at least one tetrahydropyran nucleoside analog of Formula VII:

Bx is a nucleobase moiety;

T₃ and T₄ are each, independently, an internucleoside linking group linking the tetrahydropyran nucleoside analog to the antisense compound or one of T₃ and T₄ is an internucleoside linking group linking the tetrahydropyran nucleoside analog to the antisense compound and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclic and tricyclic sugar and sugar surrogate ring systems are known in the art that can be used to modify nucleosides (see, e.g., review article: Leumann, J. C, Bioorganic & Medicinal Chemistry, 2002, 10, 841-854).

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following structure:

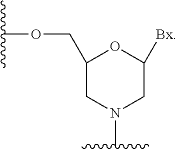

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157 Published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

Certain Nucleobases

In certain embodiments, nucleosides of the present invention comprise one or more unmodified nucleobases. In certain embodiments, nucleosides of the present invention comprise one or more modified nucleobases.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, certain of which are commonly owned with the instant application, and each of which is herein incorporated by reference in its entirety.

Certain Internucleoside Linkages

In certain embodiments, the present invention provides oligomeric compounds comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligomeric compound. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), α or β such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Certain Motifs

In certain embodiments, the present invention provides oligomeric compounds comprising oligonucleotides. In certain embodiments, such oligonucleotides comprise one or more chemical modification. In certain embodiments, chemically modified oligonucleotides comprise one or more modified nucleosides. In certain embodiments, chemically modified oligonucleotides comprise one or more modified nucleosides comprising modified sugars. In certain embodiments, chemically modified oligonucleotides comprise one or more modified nucleosides comprising one or more modified nucleobases. In certain embodiments, chemically modified oligonucleotides comprise one or more modified internucleoside linkages. In certain embodiments, the chemically modifications (sugar modifications, nucleobase modifications, and/or linkage modifications) define a pattern or motif. In certain embodiments, the patterns of chemical modifications of sugar moieties, internucleoside linkages, and nucleobases are each independent of one another. Thus, an oligonucleotide may be described by its sugar modification motif, internucleoside linkage motif and/or nucleobase modification motif (as used herein, nucleobase modification motif describes the chemical modifications to the nucleobases independent of the sequence of nucleobases).

Certain Sugar Motifs

In certain embodiments, oligonucleotides comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having a gapmer sugar modification motif, which comprises two external regions or “wings” and an internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar modification motifs of the two wings are the same as one another (symmetric gapmer). In certain embodiments, the sugar modification motifs of the 5′-wing differs from the sugar modification motif of the 3′-wing (asymmetric gapmer). In certain embodiments, oligonucleotides comprise 2′-MOE modified nucleosides in the wings and 2′-F modified nucleosides in the gap.

In certain embodiments, oligonucleotides are fully modified. In certain such embodiments, oligonucleotides are uniformly modified. In certain embodiments, oligonucleotides are uniform 2′-MOE. In certain embodiments, oligonucleotides are uniform 2′-F. In certain embodiments, oligonucleotides are uniform morpholino. In certain embodiments, oligonucleotides are uniform BNA. In certain embodiments, oligonucleotides are uniform LNA. In certain embodiments, oligonucleotides are uniform cEt.

In certain embodiments, oligonucleotides comprise a uniformly modified region and additional nucleosides that are unmodified or differently modified. In certain embodiments, the uniformly modified region is at least 5, 10, 15, or 20 nucleosides in length. In certain embodiments, the uniform region is a 2′-MOE region. In certain embodiments, the uniform region is a 2′-F region. In certain embodiments, the uniform region is a morpholino region. In certain embodiments, the uniform region is a BNA region. In certain embodiments, the uniform region is a LNA region. In certain embodiments, the uniform region is a cEt region.

In certain embodiments, the oligonucleotide does not comprise more than 4 contiguous unmodified 2′-deoxynucleosides. In certain circumstances, antisesense oligonucleotides comprising more than 4 contiguous 2′-deoxynucleosides activate RNase H, resulting in cleavage of the target RNA. In certain embodiments, such cleavage is avoided by not having more than 4 contiguous 2′-deoxynucleosides, for example, where alteration of splicing and not cleavage of a target RNA is desired.

Certain Internucleoside Linkage Motifs

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif, as described above for sugar modification motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The sugar modification motif is independently selected, so such oligonucleotides having a gapped internucleoside linkage motif may or may not have a gapped sugar modification motif and if it does have a gapped sugar motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

Certain Nucleobase Modification Motifs

In certain embodiments, oligonucleotides comprise chemical modifications to nucleobases arranged along the oligonucleotide or region thereof in a defined pattern or nucleobases modification motif. In certain such embodiments, nucleobase modifications are arranged in a gapped motif. In certain embodiments, nucleobase modifications are arranged in an alternating motif. In certain embodiments, each nucleobase is modified. In certain embodiments, none of the nucleobases is chemically modified.

In certain embodiments, oligonucleotides comprise a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5′-end of the oligonucleotide. In certain embodiments the block is within 3 nucleotides of the 5′-end of the oligonucleotide.

In certain embodiments, nucleobase modifications are a function of the natural base at a particular position of an oligonucleotide. For example, in certain embodiments each purine or each pyrimidine in an oligonucleotide is modified. In certain embodiments, each adenine is modified. In certain embodiments, each guanine is modified. In certain embodiments, each thymine is modified. In certain embodiments, each cytosine is modified. In certain embodiments, each uracil is modified.

In certain embodiments, some, all, or none of the cytosine moieties in an oligonucleotide are 5-methyl cytosine moieties. Herein, 5-methyl cytosine is not a “modified nucleobase.” Accordingly, unless otherwise indicated, unmodified nucleobases include both cytosine residues having a 5-methyl and those lacking a 5 methyl. In certain embodiments, the methylation state of all or some cytosine nucleobases is specified.

Certain Overall Lengths

In certain embodiments, the present invention provides oligomeric compounds including oligonucleotides of any of a variety of ranges of lengths. In certain embodiments, the invention provides oligomeric compounds or oligonucleotides consisting of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≦Y. For example, in certain embodiments, the invention provides oligomeric compounds which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents. In certain embodiments, a gapmer oligonucleotide has any of the above lengths.

One of skill in the art will appreciate that certain lengths may not be possible for certain motifs. For example: a gapmer having a 5′-wing region consisting of four nucleotides, a gap consisting of at least six nucleotides, and a 3′-wing region consisting of three nucleotides cannot have an overall length less than 13 nucleotides. Thus, one would understand that the lower length limit is 13 and that the limit of 10 in “10-20” has no effect in that embodiment.

Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range. For example, an oligonucleotide consisting of 20-25 linked nucleosides comprising a 5′-wing consisting of 5 linked nucleosides; a 3′-wing consisting of 5 linked nucleosides and a central gap consisting of 10 linked nucleosides (5+5+10=20) may have up to 5 nucleosides that are not part of the 5′-wing, the 3′-wing, or the gap (before reaching the overall length limitation of 25). Such additional nucleosides may be 5′ of the 5′-wing and/or 3′ of the 3′ wing.

Certain Oligonucleotides

In certain embodiments, oligonucleotides of the present invention are characterized by their sugar motif, internucleoside linkage motif, nucleobase modification motif and overall length. In certain embodiments, such parameters are each independent of one another. Thus, each internucleoside linkage of an oligonucleotide having a gapmer sugar motif may be modified or unmodified and may or may not follow the gapmer modification pattern of the sugar modifications. Thus, the internucleoside linkages within the wing regions of a sugar-gapmer may be the same or different from one another and may be the same or different from the internucleoside linkages of the gap region. Likewise, such sugar-gapmer oligonucleotides may comprise one or more modified nucleobase independent of the gapmer pattern of the sugar modifications. Herein if a description of an oligonucleotide or oligomeric compound is silent with respect to one or more parameter, such parameter is not limited. Thus, an oligomeric compound described only as having a gapmer sugar motif without further description may have any length, internucleoside linkage motif, and nucleobase modification motif. Unless otherwise indicated, all chemical modifications are independent of nucleobase sequence.

Certain Conjugate Groups

In certain embodiments, oligomeric compounds are modified by attachment of one or more conjugate groups. In general, conjugate groups modify one or more properties of the attached oligomeric compound including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In certain embodiments, conjugate groups are directly attached to oligonucleotides in oligomeric compounds. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some nonlimiting examples of conjugate linking moieties include pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an oligomeric compound. In certain embodiments, conjugate groups are near the 3′-end. In certain embodiments, conjugates are attached at the 3′end of an oligomeric compound, but before one or more terminal group nucleosides. In certain embodiments, conjugate groups are placed within a terminal group.

In certain embodiments, the present invention provides oligomeric compounds. In certain embodiments, oligomeric compounds comprise an oligonucleotide. In certain embodiments, an oligomeric compound comprises an oligonucleotide and one or more conjugate and/or terminal groups. Such conjugate and/or terminal groups may be added to oligonucleotides having any of the chemical motifs discussed above. Thus, for example, an oligomeric compound comprising an oligonucleotide having region of alternating nucleosides may comprise a terminal group.

### Antisense Compounds

In certain embodiments, oligomeric compounds of the present invention are antisense compounds. Such antisense compounds are capable of hybridizing to a target nucleic acid, resulting in at least one antisense activity. In certain embodiments, antisense compounds specifically hybridize to one or more target nucleic acid. In certain embodiments, a specifically hybridizing antisense compound has a nucleobase sequence comprising a region having sufficient complementarity to a target nucleic acid to allow hybridization and result in antisense activity and insufficient complementarity to any non-target so as to avoid non-specific hybridization to any non-target nucleic acid sequences under conditions in which specific hybridization is desired (e.g., under physiological conditions for in vivo or therapeutic uses, and under conditions in which assays are performed in the case of in vitro assays).

In certain embodiments, the present invention provides antisense compounds comprising oligonucleotides that are fully complementary to the target nucleic acid over the entire length of the oligonucleotide. In certain embodiments, oligonucleotides are 99% complementary to the target nucleic acid. In certain embodiments, oligonucleotides are 95% complementary to the target nucleic acid. In certain embodiments, such oligonucleotides are 90% complementary to the target nucleic acid.

In certain embodiments, such oligonucleotides are 85% complementary to the target nucleic acid. In certain embodiments, such oligonucleotides are 80% complementary to the target nucleic acid. In certain embodiments, an antisense compound comprises a region that is fully complementary to a target nucleic acid and is at least 80% complementary to the target nucleic acid over the entire length of the oligonucleotide. In certain such embodiments, the region of full complementarity is from 6 to 14 nucleobases in length.

In certain embodiments antisense compounds and antisense oligonucleotides comprise single-strand compounds. In certain embodiments antisense compounds and antisense oligonucleotides comprise double-strand compounds.

Certain Pathways and Mechanisms Associated with Neuronal Ceroid Lipofuscinoses

Neuronal ceroid lipofuscinoses (NCL) is the general name for a family of neurodegenerative disorders that result from excessive accumulation of lipopigments, e.g. lipofuscin, in the body's tissues. Juvenile neuronal ceroid lipofuscinosis (JNCL), also known as Batten Disease, is the most common of the NCL disorders. In certain embodiments, JNCL onset occurs between five and eight years of age and symptoms include progressive loss of motor function, seizures, vision loss, and loss of cognitive function, resulting in death before age 30.

JNCL is an autosomal recessive disorder caused by mutations of the CLN3 gene. Approximately 80% of JNCL cases result from a particular deletion of the CLN3 gene spanning exons 7 and 8 (CLN3Δ78). In certain embodiments, the CLN3Δ78 deletion causes a frameshift that results in a premature stop codon in exon 9. In certain embodiments, the truncated protein product of CLN3Δ78 is 33% of the length of the wild type CLN3 protein. In certain embodiments, the truncated protein product of CLN3Δ78 is non-functional. In certain embodiments, the truncated protein product of CLN3Δ78 is partially functional.

In certain embodiments, antisense oligonucleotides complementary to the mutant CLN3Δ78 transcript eliminate the frameshift that results in a premature stop codon in exon 9. In certain embodiments, for example, antisense oligonucleotides complementary to the mutant CLN3Δ78 transcript produce CLN3Δ78 mRNA having exons downstream of exon 9. For example, in certain embodiments, antisense oligonucleotides complementary to the mutant CLN3Δ78 transcript produce CLN3Δ78 mRNA having exons 10 and/or 11. In certain embodiments, for example, antisense oligonucleotides complementary to the mutant CLN3Δ78 transcript produce a partially deleted, but still functional CLN3Δ78 protein.

In certain embodiments, antisense oligonucleotides complementary to exon 6 of a CLN3Δ78 transcript induces skipping of exon 6 and eliminates the frameshift in CLN3Δ78 pre-mRNA that results in a premature stop codon in exon 9. In such embodiments, oligonucleotides complementary to exon 6 of a CLN3Δ78 transcript produce CLN3Δ78 mRNA without exon 6, but having the remaining exons (e.g. exons 1, 2, 3, 4, 5, 9, 10, 11 . . . ). In certain embodiments, CLN3Δ78 mRNA having the remaining exons is translated into a CLN3Δ78 protein that is partially functional. In certain embodiments, CLN3Δ78 mRNA having the remaining exons is translated into a CLN3Δ78 protein that functions in a similar manner as wild type CLN3 protein. In certain embodiments, CLN3Δ78 mRNA having the remaining exons is translated into a CLN3Δ78 protein that functions better than the truncated protein product of CLN3Δ78 containing the premature stop codon. In certain embodiments, CLN3Δ78 mRNA having the remaining exons is translated into a CLN3Δ78 protein that does not produce the excessive accumulation of lipopigments in the body tissue.

In certain embodiments, antisense oligonucleotides complementary to exon 9 of a CLN3Δ78 transcript induce skipping of exon 9 and eliminate the frameshift in CLN3Δ78 pre-mRNA that results in a premature stop codon in exon 9. In such embodiments, oligonucleotides complementary to exon 9 of a CLN3Δ78 transcript produce CLN3Δ78 mRNA without exon 9, but having the remaining exons (e.g. exons 1, 2, 3, 4, 5, 6, 10, 11 . . . ). In certain embodiments, CLN3Δ78 mRNA having the remaining exons, e.g. exons 1, 2, 3, 4, 5, 6, 10, 11 . . . , is translated into a CLN3Δ78 protein that is partially functional. In certain embodiments, CLN3Δ78 mRNA missing exons 7, 8, and 9 is translated into a CLN3Δ78 protein that functions in a similar manner as wild type CLN3 protein. In certain embodiments, CLN3Δ78 mRNA missing exons 7, 8, and 9 is translated into a CLN3Δ78 protein that functions better than the truncated protein product of CLNΔ78 containing the premature stop codon. In certain embodiments, CLN3Δ78 mRNA missing exons 7, 8, and 9 is translated into a CLN3Δ78 protein that does not produce the excessive accumulation of lipopigments in the body tissue.

Certain Target Nucleic Acids and Mechanisms

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid is a pre-mRNA. In certain embodiments, the target nucleic acid is a CLN3 transcript. In certain embodiments, the target RNA is a CLN3 pre-mRNA.

In certain embodiments, an antisense compound is complementary to a region of CLN3 pre-mRNA. In certain embodiments, an antisense compound is complementary to a region of CLN3 pre-mRNA comprising an intron-exon splice junction. In certain embodiments, an antisense compound is complementary to a region of CLN3 pre-mRNA comprising the intron-exon splice junction adjacent to exon 6. In certain embodiments, an antisense compound is complementary within a region of CLN3 pre-mRNA consisting of exon 6. In certain embodiments, an antisense compound is complementary within a region of CLN3 pre-mRNA comprising an exonic splicing silencer within an exon 6. In certain embodiments, an antisense compound is complementary to a region of CLN3 pre-mRNA comprising the intron-exon splice junction adjacent to exon 9. In certain embodiments, an antisense compound is complementary within a region of CLN3 pre-mRNA consisting of exon 9. In certain embodiments, an antisense compound is complementary within a region of CLN3 pre-mRNA comprising an exonic splicing silencer within an exon 9.

In certain embodiments, an antisense compound comprises a modified oligonucleotide consisting of 8 to 30 linked nucleosides and having a nucleobase sequence comprising a complementary region comprising at least 8 contiguous nucleobases complementary to a target region of equal length of a CLN3 transcript. In certain embodiments, the target region is within nucleobase 5082 and nucleobase 5119 of SEQ ID NO.: 2. In certain embodiments, the target region is within nucleobase 5082 and nucleobase 5099 of SEQ ID NO.: 2. In certain embodiments, the target region is within nucleobase 5053 and nucleobase 5070 of SEQ ID NO.: 2. In certain embodiments, the target region is within nucleobase 5086 and nucleobase 5103 of SEQ ID NO.: 2. In certain embodiments, the target region is within nucleobase 5090 and nucleobase 5107 of SEQ ID NO.: 2. In certain embodiments, the target region is within nucleobase 5094 and nucleobase 5111 of SEQ ID NO.: 2. In certain embodiments, the target region is within nucleobase 5098 and nucleobase 5115 of SEQ ID NO.: 2. In certain embodiments, the target region is within nucleobase 5102 and nucleobase 5119 of SEQ ID NO.: 2. In certain embodiments, the target region is within nucleobase 5126 and nucleobase 5143 of SEQ ID NO.: 2. In certain embodiments, the target region is within nucleobase 5134 and nucleobase 5155 of SEQ ID NO.: 2. In certain embodiments, the target region is within nucleobase 5134 and nucleobase 5151 of SEQ ID NO.: 2. In certain embodiments, the target region is within nucleobase 5138 and nucleobase 5155 of SEQ ID NO.: 2.

In certain embodiments, an antisense compound comprises a modified oligonucleotide consisting of 8 to 30 linked nucleosides and having a nucleobase sequence comprising a complementary region comprising at least 8 contiguous nucleobases complementary to a target region of equal length of a CLN3 transcript. In certain embodiments, the target region is within nucleobase 7366 and nucleobase 7411 of SEQ ID NO.: 2. In certain embodiments, the target region is within nucleobase 7366 and nucleobase 7383 of SEQ ID NO.: 2. In certain embodiments, the target region is within nucleobase 7370 and nucleobase 7387 of SEQ ID NO.: 2. In certain embodiments, the target region is within nucleobase 7371 and nucleobase 7388 of SEQ ID NO.: 2. In certain embodiments, the target region is within nucleobase 7387 and nucleobase 7404 of SEQ ID NO.: 2. In certain embodiments, the target region is within nucleobase 7394 and nucleobase 7411 of SEQ ID NO.: 2. In certain embodiments, the target region is within nucleobase 7454 and nucleobase 7471 of SEQ ID NO.: 2. In certain embodiments, the target region is within nucleobase 7462 and nucleobase 7483 of SEQ ID NO.: 2. In certain embodiments, the target region is within nucleobase 7462 and nucleobase 7479 of SEQ ID NO.: 2. In certain embodiments, the target region is within nucleobase 7466 and nucleobase 7483 of SEQ ID NO.: 2.

In certain embodiments, an antisense compound has a nucleobase sequence comprising SEQ ID NO. 3. In certain embodiments, an antisense compound has a nucleobase sequence comprising SEQ ID NO. 4. In certain embodiments, an antisense compound has a nucleobase sequence comprising SEQ ID NO. 5. In certain embodiments, an antisense compound has a nucleobase sequence comprising SEQ ID NO. 6. In certain embodiments, an antisense compound has a nucleobase sequence comprising SEQ ID NO. 7. In certain embodiments, an antisense compound has a nucleobase sequence comprising SEQ ID NO. 8. In certain embodiments, an antisense compound has a nucleobase sequence comprising SEQ ID NO. 9. In certain embodiments, an antisense compound has a nucleobase sequence comprising SEQ ID NO. 10. In certain embodiments, an antisense compound has a nucleobase sequence comprising SEQ ID NO. 11. In certain embodiments, an antisense compound has a nucleobase sequence comprising SEQ ID NO. 12. In certain embodiments, an antisense compound has a nucleobase sequence comprising SEQ ID NO. 13. In certain embodiments, an antisense compound has a nucleobase sequence comprising SEQ ID NO. 14. In certain embodiments, an antisense compound has a nucleobase sequence comprising SEQ ID NO. 15. In certain embodiments, an antisense compound has a nucleobase sequence comprising SEQ ID NO. 16. In certain embodiments, an antisense compound has a nucleobase sequence comprising SEQ ID NO. 17.

In certain embodiments, an antisense compound has a nucleobase sequence comprising SEQ ID NO. 18. In certain embodiments, an antisense compound has a nucleobase sequence comprising SEQ ID NO. 19. In certain embodiments, an antisense compound has a nucleobase sequence comprising SEQ ID NO. 20. In certain embodiments, an antisense compound has a nucleobase sequence comprising SEQ ID NO. 21. In certain embodiments, an antisense compound has a nucleobase sequence comprising SEQ ID NO. 22. In certain embodiments, an antisense compound has a nucleobase sequence comprising SEQ ID NO. 23. In certain embodiments, an antisense compound has a nucleobase sequence comprising SEQ ID NO. 24. In certain embodiments, an antisense compound has a nucleobase sequence comprising SEQ ID NO. 25. In certain embodiments, an antisense compound has a nucleobase sequence comprising SEQ ID NO. 26. In certain embodiments, an antisense compound has a nucleobase sequence comprising SEQ ID NO. 27. In certain embodiments, an antisense compound has a nucleobase sequence comprising SEQ ID NO. 28. In certain embodiments, an antisense compound has a nucleobase sequence comprising SEQ ID NO. 29. In certain embodiments, an antisense compound has a nucleobase sequence comprising SEQ ID NO. 30. In certain embodiments, an antisense compound has a nucleobase sequence comprising SEQ ID NO. 31. In certain embodiments, an antisense compound has a nucleobase sequence comprising SEQ ID NO. 32.

In certain embodiments, an antisense compound has a nucleobase sequence comprising SEQ ID NO. 33. In certain embodiments, an antisense compound has a nucleobase sequence comprising SEQ ID NO. 34. In certain embodiments, an antisense compound has a nucleobase sequence comprising SEQ ID NO. 35. In certain embodiments, an antisense compound has a nucleobase sequence comprising SEQ ID NO. 36. In certain embodiments, an antisense compound has a nucleobase sequence comprising SEQ ID NO. 37. In certain embodiments, an antisense compound has a nucleobase sequence comprising SEQ ID NO. 38. In certain embodiments, an antisense compound has a nucleobase sequence comprising SEQ ID NO. 39. In certain embodiments, an antisense compound has a nucleobase sequence comprising SEQ ID NO. 40. In certain embodiments, an antisense compound has a nucleobase sequence comprising SEQ ID NO. 41. In certain embodiments, an antisense compound has a nucleobase sequence comprising SEQ ID NO. 42. In certain embodiments, an antisense compound has a nucleobase sequence comprising SEQ ID NO. 43. In certain embodiments, an antisense compound has a nucleobase sequence comprising SEQ ID NO. 44. In certain embodiments, an antisense compound has a nucleobase sequence comprising SEQ ID NO. 45. In certain embodiments, an antisense compound has a nucleobase sequence comprising SEQ ID NO. 46. In certain embodiments, an antisense compound has a nucleobase sequence comprising SEQ ID NO. 47.

In certain embodiments, an antisense compound has a nucleobase sequence comprising SEQ ID NO. 48. In certain embodiments, an antisense compound has a nucleobase sequence comprising SEQ ID NO. 49. In certain embodiments, an antisense compound has a nucleobase sequence comprising SEQ ID NO. 50. In certain embodiments, an antisense compound has a nucleobase sequence comprising SEQ ID NO. 51. In certain embodiments, an antisense compound has a nucleobase sequence comprising SEQ ID NO. 52. In certain embodiments, an antisense compound has a nucleobase sequence comprising SEQ ID NO. 53. In certain embodiments, an antisense compound has a nucleobase sequence comprising SEQ ID NO. 54. In certain embodiments, an antisense compound has a nucleobase sequence comprising SEQ ID NO. 55. In certain embodiments, an antisense compound has a nucleobase sequence comprising SEQ ID NO. 56. In certain embodiments, an antisense compound has a nucleobase sequence comprising SEQ ID NO. 57. In certain embodiments, an antisense compound has a nucleobase sequence comprising SEQ ID NO. 58. In certain embodiments, an antisense compound has a nucleobase sequence comprising SEQ ID NO. 59. In certain embodiments, an antisense compound has a nucleobase sequence comprising SEQ ID NO. 60.

In certain embodiments, an antisense compound has a nucleobase sequence consisting of SEQ ID NO. 3. In certain embodiments, an antisense compound has a nucleobase sequence consisting of SEQ ID NO. 4. In certain embodiments, an antisense compound has a nucleobase sequence consisting of SEQ ID NO. 5. In certain embodiments, an antisense compound has a nucleobase sequence consisting of SEQ ID NO. 6. In certain embodiments, an antisense compound has a nucleobase sequence consisting of SEQ ID NO. 7. In certain embodiments, an antisense compound has a nucleobase sequence consisting of SEQ ID NO. 8. In certain embodiments, an antisense compound has a nucleobase sequence consisting of SEQ ID NO. 9. In certain embodiments, an antisense compound has a nucleobase sequence consisting of SEQ ID NO. 10. In certain embodiments, an antisense compound has a nucleobase sequence consisting of SEQ ID NO. 11. In certain embodiments, an antisense compound has a nucleobase sequence consisting of SEQ ID NO. 12. In certain embodiments, an antisense compound has a nucleobase sequence consisting of SEQ ID NO. 13. In certain embodiments, an antisense compound has a nucleobase sequence consisting of SEQ ID NO. 14. In certain embodiments, an antisense compound has a nucleobase sequence consisting of SEQ ID NO. 15. In certain embodiments, an antisense compound has a nucleobase sequence consisting of SEQ ID NO. 16. In certain embodiments, an antisense compound has a nucleobase sequence consisting of SEQ ID NO. 17.

In certain embodiments, an antisense compound has a nucleobase sequence consisting of SEQ ID NO. 18. In certain embodiments, an antisense compound has a nucleobase sequence consisting of SEQ ID NO. 19. In certain embodiments, an antisense compound has a nucleobase sequence consisting of SEQ ID NO. 20. In certain embodiments, an antisense compound has a nucleobase sequence consisting of SEQ ID NO. 21. In certain embodiments, an antisense compound has a nucleobase sequence consisting of SEQ ID NO. 22. In certain embodiments, an antisense compound has a nucleobase sequence consisting of SEQ ID NO. 23. In certain embodiments, an antisense compound has a nucleobase sequence consisting of SEQ ID NO. 24. In certain embodiments, an antisense compound has a nucleobase sequence consisting of SEQ ID NO. 25. In certain embodiments, an antisense compound has a nucleobase sequence consisting of SEQ ID NO. 26. In certain embodiments, an antisense compound has a nucleobase sequence consisting of SEQ ID NO. 27. In certain embodiments, an antisense compound has a nucleobase sequence consisting of SEQ ID NO. 28. In certain embodiments, an antisense compound has a nucleobase sequence consisting of SEQ ID NO. 29. In certain embodiments, an antisense compound has a nucleobase sequence consisting of SEQ ID NO. 30. In certain embodiments, an antisense compound has a nucleobase sequence consisting of SEQ ID NO. 31. In certain embodiments, an antisense compound has a nucleobase sequence consisting of SEQ ID NO. 32.

In certain embodiments, an antisense compound has a nucleobase sequence consisting of SEQ ID NO. 33. In certain embodiments, an antisense compound has a nucleobase sequence consisting of SEQ ID NO. 34. In certain embodiments, an antisense compound has a nucleobase sequence consisting of SEQ ID NO. 35. In certain embodiments, an antisense compound has a nucleobase sequence consisting of SEQ ID NO. 36. In certain embodiments, an antisense compound has a nucleobase sequence consisting of SEQ ID NO. 37. In certain embodiments, an antisense compound has a nucleobase sequence consisting of SEQ ID NO. 38. In certain embodiments, an antisense compound has a nucleobase sequence consisting of SEQ ID NO. 39. In certain embodiments, an antisense compound has a nucleobase sequence consisting of SEQ ID NO. 40. In certain embodiments, an antisense compound has a nucleobase sequence consisting of SEQ ID NO. 41. In certain embodiments, an antisense compound has a nucleobase sequence consisting of SEQ ID NO. 42. In certain embodiments, an antisense compound has a nucleobase sequence consisting of SEQ ID NO. 43. In certain embodiments, an antisense compound has a nucleobase sequence consisting of SEQ ID NO. 44. In certain embodiments, an antisense compound has a nucleobase sequence consisting of SEQ ID NO. 45. In certain embodiments, an antisense compound has a nucleobase sequence consisting of SEQ ID NO. 46. In certain embodiments, an antisense compound has a nucleobase sequence consisting of SEQ ID NO. 47.

In certain embodiments, an antisense compound has a nucleobase sequence consisting of SEQ ID NO. 48. In certain embodiments, an antisense compound has a nucleobase sequence consisting of SEQ ID NO. 49. In certain embodiments, an antisense compound has a nucleobase sequence consisting of SEQ ID NO. 50. In certain embodiments, an antisense compound has a nucleobase sequence consisting of SEQ ID NO. 51. In certain embodiments, an antisense compound has a nucleobase sequence consisting of SEQ ID NO. 52. In certain embodiments, an antisense compound has a nucleobase sequence consisting of SEQ ID NO. 53. In certain embodiments, an antisense compound has a nucleobase sequence consisting of SEQ ID NO. 54. In certain embodiments, an antisense compound has a nucleobase sequence consisting of SEQ ID NO. 55. In certain embodiments, an antisense compound has a nucleobase sequence consisting of SEQ ID NO. 56. In certain embodiments, an antisense compound has a nucleobase sequence consisting of SEQ ID NO. 57. In certain embodiments, an antisense compound has a nucleobase sequence consisting of SEQ ID NO. 58. In certain embodiments, an antisense compound has a nucleobase sequence consisting of SEQ ID NO. 59. In certain embodiments, an antisense compound has a nucleobase sequence consisting of SEQ ID NO. 60.

In certain embodiments, an antisense oligonucleotide modulates splicing of a pre-mRNA. In certain embodiments, an antisense oligonucleotide modulates splicing a CLN3 pre-mRNA. In certain embodiments, an antisense oligonucleotide increases the amount of CLN3 mRNA. In certain embodiments, an antisense oligonucleotide increases the exclusion of exon 6 in CLN3 mRNA. In certain embodiments, an antisense oligonucleotide decreases the inclusion of exon 6 in CLN3 mRNA. In certain embodiments, an antisense oligonucleotide increases the exclusion of exon 9 in CLN3 mRNA. In certain embodiments, an antisense oligonucleotide decreases the inclusion of exon 9 in CLN3 mRNA.

In certain embodiments, an antisense oligonucleotide comprises ISIS NO. 616709 (SEQ ID NO: 20). In certain embodiments, an antisense oligonucleotide consists of ISIS NO. 616709.

In certain embodiments, an antisense oligonucleotide promotes skipping of exon 6. In certain embodiments, an antisense oligonucleotide promotes skipping of exon 6 of a CLN3Δ78 transcript. In certain embodiments, an antisense oligonucleotide promotes skipping of exon 9. In certain embodiments, an antisense oligonucleotide promotes skipping of exon 9 of a CLN3Δ78 transcript.

In certain embodiments, an antisense oligonucleotide comprises the nucleobase sequence and modification motif of ISIS NO. 688555. In certain embodiments, an antisense oligonucleotide consists of the nucleobase sequence and modification motif of ISIS NO. 688555. In certain embodiments, an antisense oligonucleotide comprises the nucleobase sequence and modification motif of ISIS NO. 688559. In certain embodiments, an antisense oligonucleotide consists of the nucleobase sequence and modification motif of ISIS NO. 688559. In certain embodiments, an antisense oligonucleotide comprises the nucleobase sequence and modification motif of ISIS NO. 688595. In certain embodiments, an antisense oligonucleotide consists of the nucleobase sequence and modification motif of ISIS NO. 688595.

In certain embodiments, an antisense compound has a nucleobase sequence consisting of SEQ ID NO. 63. In certain embodiments, an antisense compound has a nucleobase sequence consisting of SEQ ID NO. 64. In certain embodiments, an antisense compound has a nucleobase sequence consisting of SEQ ID NO. 65. In certain embodiments, an antisense compound has a nucleobase sequence consisting of SEQ ID NO. 66. In certain embodiments, an antisense compound has a nucleobase sequence consisting of SEQ ID NO. 67. In certain embodiments, an antisense compound has a nucleobase sequence consisting of SEQ ID NO. 68. In certain embodiments, an antisense compound has a nucleobase sequence consisting of SEQ ID NO. 69. In certain embodiments, an antisense compound has a nucleobase sequence consisting of SEQ ID NO. 70. In certain embodiments, an antisense compound has a nucleobase sequence consisting of SEQ ID NO. 71. In certain embodiments, an antisense compound has a nucleobase sequence consisting of SEQ ID NO. 72. In certain embodiments, an antisense compound has a nucleobase sequence consisting of SEQ ID NO. 73. In certain embodiments, an antisense compound has a nucleobase sequence consisting of SEQ ID NO. 74. In certain embodiments, an antisense compound has a nucleobase sequence consisting of SEQ ID NO. 75. In certain embodiments, an antisense compound has a nucleobase sequence consisting of SEQ ID NO. 76. In certain embodiments, an antisense compound has a nucleobase sequence consisting of SEQ ID NO. 77. In certain embodiments, an antisense compound has a nucleobase sequence consisting of SEQ ID NO. 78. In certain embodiments, an antisense compound has a nucleobase sequence consisting of SEQ ID NO. 79. In certain embodiments, an antisense compound has a nucleobase sequence consisting of SEQ ID NO. 80. In certain embodiments, an antisense compound has a nucleobase sequence consisting of SEQ ID NO. 81. In certain embodiments, an antisense compound has a nucleobase sequence consisting of SEQ ID NO. 82. In certain embodiments, an antisense compound has a nucleobase sequence consisting of SEQ ID NO. 83 In certain embodiments, an antisense compound has a nucleobase sequence consisting of SEQ ID NO. 84. In certain embodiments, an antisense compound has a nucleobase sequence consisting of SEQ ID NO. 85. In certain embodiments, an antisense compound has a nucleobase sequence consisting of SEQ ID NO. 86. In certain embodiments, an antisense compound has a nucleobase sequence consisting of SEQ ID NO. 87. In certain embodiments, an antisense compound has a nucleobase sequence consisting of SEQ ID NO. 88. In certain embodiments, an antisense compound has a nucleobase sequence consisting of SEQ ID NO. 89. In certain embodiments, an antisense compound has a nucleobase sequence consisting of SEQ ID NO. 90.

In certain embodiments, an antisense compound has a nucleobase sequence consisting of SEQ ID NO. 91. In certain embodiments, an antisense compound has a nucleobase sequence consisting of SEQ ID NO. 92. In certain embodiments, an antisense compound has a nucleobase sequence consisting of SEQ ID NO. 93. In certain embodiments, an antisense compound has a nucleobase sequence consisting of SEQ ID NO. 94. In certain embodiments, an antisense compound has a nucleobase sequence consisting of SEQ ID NO. 95. In certain embodiments, an antisense compound has a nucleobase sequence consisting of SEQ ID NO. 96. In certain embodiments, an antisense compound has a nucleobase sequence consisting of SEQ ID NO. 97. In certain embodiments, an antisense compound has a nucleobase sequence consisting of SEQ ID NO. 98. In certain embodiments, an antisense compound has a nucleobase sequence consisting of SEQ ID NO. 99. In certain embodiments, an antisense compound has a nucleobase sequence consisting of SEQ ID NO. 100. In certain embodiments, an antisense compound has a nucleobase sequence consisting of SEQ ID NO. 101.

In certain embodiments, an antisense compound has a nucleobase sequence consisting of SEQ ID NO. 102. In certain embodiments, an antisense compound has a nucleobase sequence consisting of SEQ ID NO. 103. In certain embodiments, an antisense compound has a nucleobase sequence consisting of SEQ ID NO. 104. In certain embodiments, an antisense compound has a nucleobase sequence consisting of SEQ ID NO. 105. In certain embodiments, an antisense compound has a nucleobase sequence consisting of SEQ ID NO. 106. In certain embodiments, an antisense compound has a nucleobase sequence consisting of SEQ ID NO. 107. In certain embodiments, an antisense compound has a nucleobase sequence consisting of SEQ ID NO. 108. In certain embodiments, an antisense compound has a nucleobase sequence consisting of SEQ ID NO. 109. In certain embodiments, an antisense compound has a nucleobase sequence consisting of SEQ ID NO. 110.

In certain embodiments, an antisense compound has a nucleobase sequence consisting of SEQ ID NO. 111. In certain embodiments, an antisense compound has a nucleobase sequence consisting of SEQ ID NO. 112. In certain embodiments, an antisense compound has a nucleobase sequence consisting of SEQ ID NO. 113. In certain embodiments, an antisense compound has a nucleobase sequence consisting of SEQ ID NO. 114. In certain embodiments, an antisense compound has a nucleobase sequence consisting of SEQ ID NO. 115. In certain embodiments, an antisense compound has a nucleobase sequence consisting of SEQ ID NO. 116. In certain embodiments, an antisense compound has a nucleobase sequence consisting of SEQ ID NO. 117. In certain embodiments, an antisense compound has a nucleobase sequence consisting of SEQ ID NO. 118. In certain embodiments, an antisense compound has a nucleobase sequence consisting of SEQ ID NO. 119. In certain embodiments, an antisense compound has a nucleobase sequence consisting of SEQ ID NO. 120. In certain embodiments, an antisense compound has a nucleobase sequence consisting of SEQ ID NO. 121. In certain embodiments, an antisense compound has a nucleobase sequence consisting of SEQ ID NO. 122. In certain embodiments, an antisense compound has a nucleobase sequence consisting of SEQ ID NO. 123. In certain embodiments, an antisense compound has a nucleobase sequence consisting of SEQ ID NO. 124. In certain embodiments, an antisense compound has a nucleobase sequence consisting of SEQ ID NO. 125. In certain embodiments, an antisense compound has a nucleobase sequence consisting of SEQ ID NO. 126. In certain embodiments, an antisense compound has a nucleobase sequence consisting of SEQ ID NO. 127.

### Certain Pharmaceutical Compositions

In certain embodiments, the present invention provides pharmaceutical compositions comprising one or more antisense compound. In certain embodiments, such pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one or more antisense compound and sterile water. In certain embodiments, the sterile saline is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile phosphate-buffered saline (PBS). In certain embodiments, the sterile saline is pharmaceutical grade PBS.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising antisense compounds encompass any pharmaceutically acceptable salts, esters, or salts of such esters. In certain embodiments, pharmaceutical compositions comprising antisense compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligomeric compound which are cleaved by endogenous nucleases within the body, to form the active antisense oligomeric compound.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions provided herein comprise one or more modified oligonucleotides and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition provided herein comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition provided herein comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition provided herein is prepared for oral administration. In certain embodiments, pharmaceutical compositions are prepared for buccal administration.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition provided herein comprises an oligonucleotide in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more modified oligonucleotide provided herein is formulated as a prodrug. In certain embodiments, upon in vivo administration, a prodrug is chemically converted to the biologically, pharmaceutically or therapeutically more active form of an oligonucleotide. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, the present invention provides compositions and methods for reducing the amount or activity of a target nucleic acid in a cell. In certain embodiments, the cell is in an animal. In certain embodiments, the animal is a mammal. In certain embodiments, the animal is a rodent. In certain embodiments, the animal is a primate. In certain embodiments, the animal is a non-human primate. In certain embodiments, the animal is a human.

In certain embodiments, the present invention provides methods of administering a pharmaceutical composition comprising an oligomeric compound of the present invention to an animal. Suitable administration routes include, but are not limited to, oral, rectal, transmucosal, intestinal, enteral, topical, suppository, through inhalation, intrathecal, intracerebroventricular, intraperitoneal, intranasal, intratumoral, and parenteral (e.g., intravenous, intramuscular, intramedullary, and subcutaneous). In certain embodiments, pharmaceutical intrathecals are administered to achieve local rather than systemic exposures. For example, pharmaceutical compositions may be injected directly in the area of desired effect (e.g., into the ears).

In certain embodiments, a pharmaceutical composition is administered to an animal having at least one symptom associated with Batten Disease. In certain embodiments, such administration results in amelioration of at least one symptom. In certain embodiments, administration of a pharmaceutical composition to an animal results in an increase in functional CLN3 protein in a cell. In certain embodiments, the administration of certain antisense oligonucleotides delays the onset of Batten Disease. In certain embodiments, the administration of certain antisense oligonucleotides prevents the onset of Batten Disease. In certain embodiments, the administration of certain antisense oligonucleotides rescues cellular phenotype.

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified or naturally occurring bases, such as “AT^{me}CGAUCG,” wherein ^{me}C indicates a cytosine base comprising a methyl group at the 5-position.

### EXAMPLES

The following examples illustrate certain embodiments of the present invention and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Antisense Oligonucleotide Synthesis

The preparation of nucleoside phosphoramidites is performed following procedures that are extensively illustrated in the art such as but not limited to U.S. Pat. No. 6,426,220 and published PCT application WO 02/36743.

### Oligonucleotide and Oligonucleoside Synthesis

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270.

Alkyl phosphonate oligonucleotides are prepared as described in U.S. Pat. No. 4,469,863.

3′-Deoxy-3′-methylene phosphonate oligonucleotides are prepared as described in U.S. Pat. No. 5,610,289 or 5,625,050.

Phosphoramidite oligonucleotides are prepared as described in U.S. Pat. No. 5,256,775 or U.S. Pat. No. 5,366,878.

Alkylphosphonothioate oligonucleotides are prepared as described in published PCT applications PCT/US94/00902 and PCT/US93/06976 (published as WO 94/17093 and WO 94/02499, respectively).

3′-Deoxy-3′-amino phosphoramidate oligonucleotides are prepared as described in U.S. Pat. No. 5,476,925.

Phosphotriester oligonucleotides are prepared as described in U.S. Pat. No. 5,023,243.

Borano phosphate oligonucleotides are prepared as described in U.S. Pat. Nos. 5,130,302 and 5,177,198.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone oligomeric compounds having, for instance, alternating MMI and P═O or P═S linkages are prepared as described in U.S. Pat. Nos. 5,378,825, 5,386,023, 5,489,677, 5,602,240 and 5,610,289.

Formacetal and thioformacetal linked oligonucleosides are prepared as described in U.S. Pat. Nos. 5,264,562 and 5,264,564.

Ethylene oxide linked oligonucleosides are prepared as described in U.S. Pat. No. 5,223,618.

### Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32 +/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Oligonucleotide Synthesis—96 Well Plate Format

Oligonucleotides can be synthesized via solid phase P(III) phosphoramidite chemistry on an automated synthesizer capable of assembling 96 sequences simultaneously in a 96-well format. Phosphodiester internucleotide linkages are afforded by oxidation with aqueous iodine. Phosphorothioate internucleotide linkages are generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites are purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides are cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

### Oligonucleotide Analysis Using 96-Well Plate Format

The concentration of oligonucleotide in each well is assessed by dilution of samples and UV absorption spectroscopy. The full-length integrity of the individual products is evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition is confirmed by mass analysis of the oligomeric compounds utilizing electrospray-mass spectroscopy. All assay test plates are diluted from the master plate using single and multi-channel robotic pipettors. Plates are judged to be acceptable if at least 85% of the oligomeric compounds on the plate are at least 85% full length.

### Example 2: Expression of CLN3Δ678 and CLN3Δ78 In Vitro

Plasmids comprising a C-terminal FLAG-tagged WT or mutant CLN3 gene were prepared using standard molecular biology techniques. The mutant CLN3 genes contained a deletion of exons 6, 7, and 8 (CLN3Δ678) or deletion of exons 7, 8, and 9 (CLN3Δ789). The WT and mutant CLN3 genes were under control of the CMV promoter. HeLa cells were transfected with plasmids containing WT CLN3, CLN3Δ78, CLN3Δ678, or empty plasmids (mock) using standard methods. 48 hours after transfection, the cells were fixed and stained with anti-FLAG M2 FITC antibody (F4049, Sigma), mounted with Prolong antifade with DAPI (Invitrogen) and visualized under an Olympus FV10i confocal microscope.

While none of the mock or CLN3Δ78 transfected cells exhibited FITC fluorescence, many of the WT CLN3 and CLN3Δ678 transfected cells were fluorescent in the FITC channel. These results indicate that the CLN3Δ78 protein was not visibly expressed, but the wild type CLN3 and CLN3Δ678 proteins were visibly expressed.

### Example 3: Antisense Modulation of CLN3 Transcript Splicing

An antisense oligonucleotide targeted to the exon 6/intron 6 junction of human CLN3 pre-mRNA (MO-ASO ACACAGAACCACACACTCACCACAC, SEQ ID NO: 3) was synthesized by Gene Tools, LLC, and tested for its ability to modulate splicing of CLN3. The ASO targets the exon 6/intron 6 junction of the complement of GENBANK accession number NT_010393.16 truncated from nucleotides 28427600 to 28444620 (SEQ ID NO: 1). The ASO is a uniform morpholino ASO.

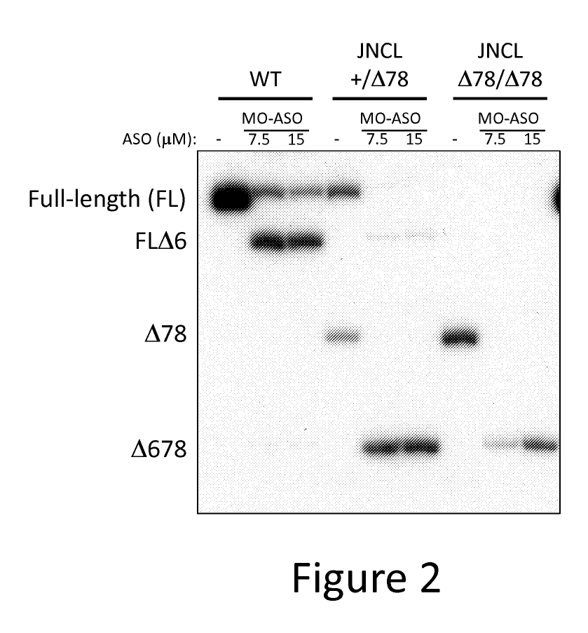

0 μM, 7.5 μM, 15 μM of the morpholino ASO targeted to the exon 6/intron 6 junction (MO-ASO) and Endo-porter transfection reagent (Gene Tools, LLC) were added to fibroblast cells of a JNCL patient homozygous for the CLN3Δ78 mutation, a heterozygous carrier of the CLN3Δ78 mutation, and a family member of the JNCL patient who is homozygous for WT CLN3. After 48 hours, total RNA was then isolated from the cells and CLN3 mRNA was detected via radioactive RT-PCR and PAGE. The gel is shown in FIG. 2. Bands were quantitated by densitometry analysis using Image J software, and the results are shown in Table 1 below. In Table 1, ‘Ht’ means heterozygote, and ‘Hm’ means homozygote.

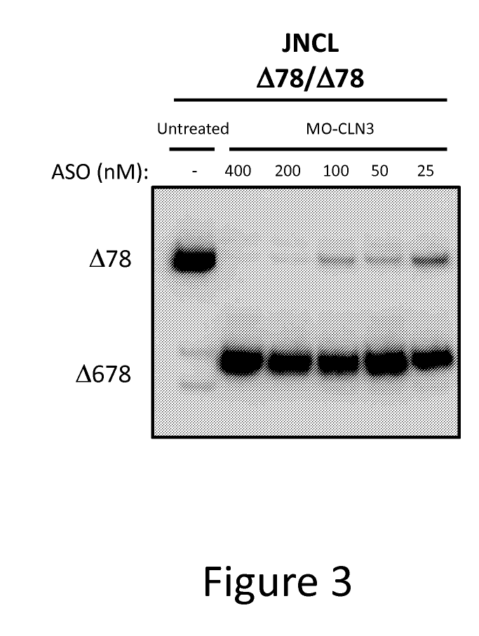

In a similar experiment, increasing concentrations of MO-ASO were incubated with homozygous CLN3Δ78/Δ78 patient fibroblasts. The lowest dose of 25 nM induced % exon 6 skipping, and the highest doses tested induced % exon 6 skipping. The results are shown in Table 2 below, and the gel is shown in FIG. 3.

TABLE 1
Modulation of Splicing of CLN3 pre-mRNA by MO-ASO as
analyzed by RT-PCR

| | | % of all Isoforms |
|---|---|---|
| Genotype | MO-ASO Concentration | Lacking Exon 6 |
| WT | 0 μM (untreated control) | 0.0 |
| WT | 7.5 μM | 67.9 |
| WT | 15 μM | 73.7 |
| Ht | 0 μM (untreated control) | 0.0 |
| Ht | 7.5 μM | 92.8 |
| Ht | 15 μM | 93.4 |
| Hm | 0 μM (untreated control) | 0.0 |
| Hm | 7.5 μM | 74.6 |
| Hm | 15 μM | 93.3 |

TABLE 2
MO-ASO Dose Response Analysis of CLN3 pre-mRNA
Splicing in CLN3Δ78/Δ78 cells

| MO-ASO concentration | % of all Isoforms Lacking Exon 6 |
|---|---|
| 0 nM (untreated control) | 4.0 |
| 25 nM | 82.9 |
| 50 nM | 94.5 |
| 100 nM | 89.7 |
| 200 nM | 98.4 |
| 400 nM | 99.4 |

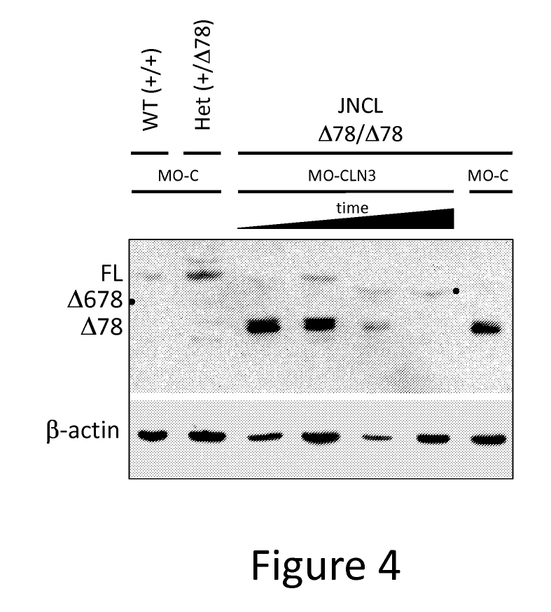

CLN3 protein levels were measured in a similar experiment in which a single concentration (7.5 μM) of the MO-ASO or a control morpholino oligonucleotide (MO-C, AGCTGATCATATTCTACCTGGTGCT SEQ ID NO: 62) was used and protein levels in homozygous mutant CLN3Δ78 cells were analyzed at the following 0, 56, 144, or 192 hours following incubation. Protein levels were analyzed by western blot using a rabbit poly-clonal anti-CLN3 antibody (Proteintech) and anti-β-actin as a loading control. The blot is shown in FIG. 4.

### Example 4: Design of Antisense Oligonucleotides for Induction of CLN3 Exon 6 Skipping

Antisense oligonucleotides complementary to exon 6 and/or the introns flanking exon 6 of the mouse CLN3 pre-mRNA transcript were synthesized and tested for their ability to modulate splicing of CLN3 by skipping exon 6. The oligonucleotides in Table 3 below are uniformly 2′-MOE modified, and all internucleoside linkages are phosphorothioate linkages. The cytosine bases are 5-methylcytosine. The ASOs target the complement of GENBANK accession number NT_039433.8, truncated from nucleotides 44319075 to 44333955 (SEQ ID NO: 2), and the ASO sequences are listed in Table 3 below. The start and stop sites associated with each oligonucleotide are the 5′- and 3′-positions, respectively, of the portion of SEQ ID NO: 2 that is complementary to the ASO.

TABLE 3
Antisense Oligonucleotides for Induction of CLN3 Exon 6 Skipping

| ISIS | Start | Stop | | CLN3 | |
|---|---|---|---|---|---|
| No. | Site | Site | Sequence (5′ to 3′) | Target Region | SEQ ID NO |
| 616693 | 5030 | 5047 | GAGAAGAGATGAGGAGGA | Intron 5 | 4 |
| 616694 | 5034 | 5051 | GGCTGAGAAGAGATGAGG | Intron 5/Exon 6 | 5 |
| 616695 | 5035 | 5052 | GGGCTGAGAAGAGATGAG | Intron 5/Exon 6 | 6 |
| 616696 | 5052 | 5069 | CACTGACGAGCACCCGGG | Exon 6 | 7 |
| 616697 | 5053 | 5070 | CCACTGACGAGCACCCGG | Exon 6 | 8 |
| 616698 | 5054 | 5071 | TCCACTGACGAGCACCCG | Exon 6 | 9 |
| 616699 | 5058 | 5075 | AAACTCCACTGACGAGCA | Exon 6 | 10 |
| 616700 | 5062 | 5079 | GAACAAACTCCACTGACG | Exon 6 | 11 |
| 616701 | 5066 | 5083 | AGCAGAACAAACTCCACT | Exon 6 | 12 |
| 616702 | 5070 | 5087 | TCCCAGCAGAACAAACTC | Exon 6 | 13 |
| 616703 | 5074 | 5091 | AAGCTCCCAGCAGAACAA | Exon 6 | 14 |
| 616704 | 5078 | 5095 | AACAAAGCTCCCAGCAGA | Exon 6 | 15 |
| 616705 | 5082 | 5099 | CCAGAACAAAGCTCCCAG | Exon 6 | 16 |
| 616706 | 5086 | 5103 | GCAACCAGAACAAAGCTC | Exon 6 | 17 |
| 616707 | 5090 | 5107 | GAAGGCAACCAGAACAAA | Exon 6 | 18 |
| 616708 | 5094 | 5111 | GAGAGAAGGCAACCAGAA | Exon 6 | 19 |
| 616709 | 5098 | 5115 | GACTGAGAGAAGGCAACC | Exon 6 | 20 |
| 616710 | 5102 | 5119 | CACTGACTGAGAGAAGGC | Exon 6 | 21 |
| 616711 | 5106 | 5123 | ACCCCACTGACTGAGAGA | Exon 6 | 22 |
| 616712 | 5110 | 5127 | CTTAACCCCACTGACTGA | Exon 6 | 23 |
| 616713 | 5114 | 5131 | CAGGCTTAACCCCACTGA | Exon 6 | 24 |
| 616714 | 5118 | 5135 | CACACAGGCTTAACCCCA | Exon 6 | 25 |
| 616715 | 5122 | 5139 | TCACCACACAGGCTTAAC | Exon 6/Intron 6 | 26 |
| 616716 | 5126 | 5143 | GTACTCACCACACAGGCT | Exon 6/Intron 6 | 27 |
| 616717 | 5130 | 5147 | CTTCGTACTCACCACACA | Exon 6/Intron 6 | 28 |
| 616718 | 5134 | 5151 | CCAGCTTCGTACTCACCA | Exon 6/Intron 6 | 29 |
| 616719 | 5138 | 5155 | TCTCCCAGCTTCGTACTC | Intron 6 | 30 |

### Example 5: Antisense Oligonucleotide Induction of CLN3 Exon 6 Skipping In Vitro

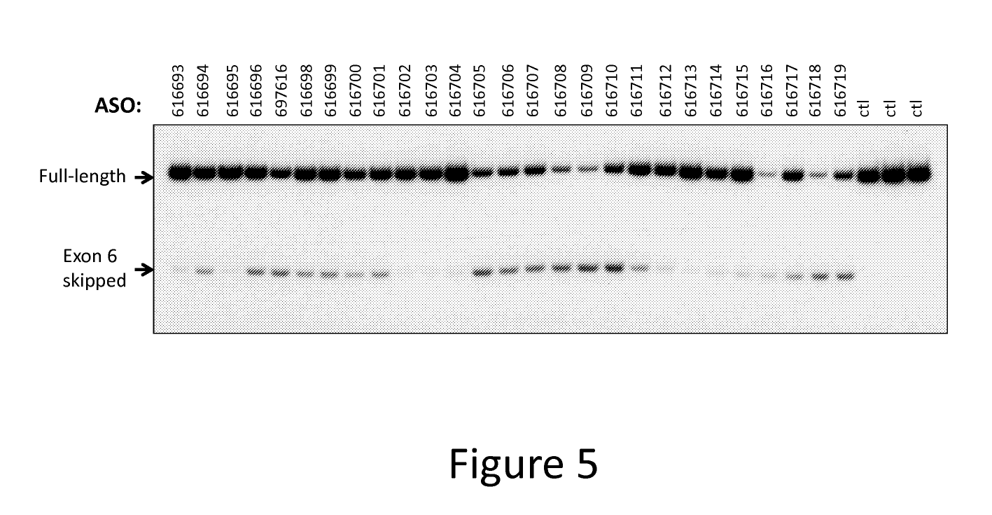

To test the ability of the antisense oligonucleotides to modulate splicing by skipping exon 6, mouse cell line 208ee, derived from C57/Bl6 mouse kidney, was transfected with 50 nM of the ASOs listed in Table 3 using Lipofectamine 2000 (Invitrogen). Untreated control cells received neither ASO nor Lipofectamine, while mock transfected cells received only Lipofectamine. After 48 hours, total RNA was collected from the cells and RT-PCR was used to identify CLN3 full-length and CLN3Δ6 transcripts. The PCR products were analyzed by PAGE and quantitated by phosphorimager analysis (Typhoon 9400, GE Healthcare). The gel is shown in FIG. 5, and the results are shown in Table 4 below as the percentage of exon 6 skipped (Δ6) transcript out of the total full-length (FL) plus exon 6 skipped transcripts. Band intensities are reported in relative fluorescence units (RFU). This example illustrates that many ASOs induce CLN3 exon 6 skipping.

TABLE 4
Induction of CLN3 Exon 6 Skipping in vitro

| | Full-length CLN3 | | Δ6/(FL + Δ6) |
|---|---|---|---|
| ISIS NO | (RFU) | CLN3Δ6 (RFU) | (%) |
| untreated control | 6,823,783 | 762 | 0.01 |
| mock | 5,188,149 | 2,110 | 0.04 |
| 616693 | 6,723,467 | 253,542 | 3.63 |
| 616694 | 5,083,972 | 703,577 | 12.16 |
| 616695 | 6,278,156 | 225,133 | 3.46 |
| 616696 | 5,488,331 | 941,697 | 14.65 |
| 616697 | 3,262,413 | 1,067,086 | 24.65 |
| 616698 | 5,125,340 | 688,277 | 11.84 |
| 616699 | 4,999,204 | 727,472 | 12.70 |
| 616700 | 3,286,457 | 523332 | 13.74 |
| 616701 | 4,054,613 | 653,463 | 13.88 |
| 616702 | 4,047,520 | 73,846 | 1.79 |
| 616703 | 4,452,892 | 96,627 | 2.12 |
| 616704 | 7,603,794 | 152,903 | 1.97 |
| 616705 | 2,177,616 | 1,254,116 | 36.54 |
| 616706 | 2,222,239 | 903,504 | 28.91 |
| 616707 | 2,432,817 | 1,009,358 | 29.32 |
| 616708 | 1,182,124 | 1,120,453 | 48.66 |
| 616709 | 894,622 | 1,501,534 | 62.66 |
| 616710 | 2,315,933 | 1,597,682 | 40.82 |
| 616711 | 4,134,353 | 640,071 | 13.41 |
| 616712 | 3,928,470 | 229,565 | 5.52 |
| 616713 | 5,499,952 | 56,355 | 1.01 |
| 616714 | 3,618,665 | 180,643 | 4.75 |
| 616715 | 5,176,194 | 235,685 | 4.35 |
| 616716 | 692,245 | 335,115 | 32.62 |
| 616717 | 2,855,386 | 512,449 | 15.22 |
| 616718 | 730,706 | 832,631 | 53.26 |
| 616719 | 1,738,032 | 856,673 | 33.02 |

### Example 6: Design of Antisense Oligonucleotides for Induction of CLN3 Exon 9 Skipping

Antisense oligonucleotides complementary to exon 9 and/or the introns flanking exon 9 of the mouse CLN3 pre-mRNA transcript were synthesized and tested for their ability to modulate splicing of CLN3 by skipping exon 9. The oligonucleotides were uniformly modified to contain 2′-MOE, and all internucleoside linkages are phosphorothioate linkages. The cytosine bases are 5-methylcytosine. The ASOs target the complement of GENBANK accession number NT_039433.8 truncated from nucleotides 44319075 to 44333955 (SEQ ID NO: 2), and the ASO sequences are listed in Table 5 below. The start and stop sites associated with each oligonucleotide are the 5′- and 3′-positions, respectively, of the portion of SEQ ID NO: 2 that is complementary to the ASO.

TABLE 5
Antisense Oligonucleotides for Induction of CLN3 Exon 9 Skipping

| | Start | Stop | | CLN3 | |
|---|---|---|---|---|---|
| 3 | Site | Site | Sequence (5′ to 3′) | Target Region | SEQ ID NO |
| 616720 | 7338 | 7355 | GGGATGGAATGGAGAAGC | Intron 8 | 31 |
| 616721 | 7342 | 7359 | AGCTGGGATGGAATGGAG | Intron 8/Exon 9 | 32 |
| 616722 | 7346 | 7363 | AAATAGCTGGGATGGAAT | Intron 8/Exon 9 | 33 |
| 616723 | 7350 | 7367 | CAAGAAATAGCTGGGATG | Intron 8/Exon 9 | 34 |
| 616724 | 7354 | 7371 | GCAACAAGAAATAGCTGG | Intron 8/Exon 9 | 35 |
| 616725 | 7358 | 7375 | GTGAGCAACAAGAAATAG | Exon 9 | 36 |
| 616726 | 7362 | 7379 | AGACGTGAGCAACAAGAA | Exon 9 | 37 |
| 616727 | 7366 | 7383 | CAGGAGACGTGAGCAACA | Exon 9 | 38 |
| 616728 | 7370 | 7387 | GGTTCAGGAGACGTGAGC | Exon 9 | 39 |
| 616729 | 7371 | 7388 | GGGTTCAGGAGACGTGAG | Exon 9 | 40 |
| 616730 | 7387 | 7404 | CCCCTCCAGGGTCCAGGG | Exon 9 | 41 |
| 616731 | 7390 | 7407 | TTTCCCCTCCAGGGTCCA | Exon 9 | 42 |
| 616732 | 7394 | 7411 | TCGTTTTCCCCTCCAGGG | Exon 9 | 43 |
| 616733 | 7398 | 7415 | TGCCTCGTTTTCCCCTCC | Exon 9 | 44 |
| 616734 | 7402 | 7419 | TCTCTGCCTCGTTTTCCC | Exon 9 | 45 |
| 616735 | 7406 | 7423 | GCAGTCTCTGCCTCGTTT | Exon 9 | 46 |
| 616736 | 7410 | 7427 | GGCAGCAGTCTCTGCCTC | Exon 9 | 47 |
| 616737 | 7414 | 7431 | GCCGGGCAGCAGTCTCTG | Exon 9 | 48 |
| 616738 | 7418 | 7435 | GGCTGCCGGGCAGCAGTC | Exon 9 | 49 |
| 616739 | 7422 | 7439 | GAGAGGCTGCCGGGCAGC | Exon 9 | 50 |
| 616740 | 7426 | 7443 | CTATGAGAGGCTGCCGGG | Exon 9 | 51 |
| 616741 | 7430 | 7447 | GTGCCTATGAGAGGCTGC | Exon 9 | 52 |
| 616742 | 7434 | 7451 | CTCGGTGCCTATGAGAGG | Exon 9 | 53 |
| 616743 | 7438 | 7455 | GGGTCTCGGTGCCTATGA | Exon 9 | 54 |
| 616744 | 7454 | 7471 | CCTGGCTTTGACTCTGGG | Exon 9/Intron 9 | 55 |
| 616745 | 7458 | 7475 | CCTACCTGGCTTTGACTC | Exon 9/Intron 9 | 56 |
| 616746 | 7462 | 7479 | GCATCCTACCTGGCTTTG | Exon 9/Intron 9 | 57 |
| 616747 | 7466 | 7483 | CTTTGCATCCTACCTGGC | Exon 9/Intron 9 | 58 |
| 616748 | 7470 | 7487 | GGGCCTTTGCATCCTACC | Exon 9/Intron 9 | 59 |
| 616749 | 7474 | 7491 | GGGAGGGCCTTTGCATCC | Intron 9 | 60 |

### Example 7: Antisense Oligonucleotide Induction of CLN3 Exon 9 Skipping In Vitro

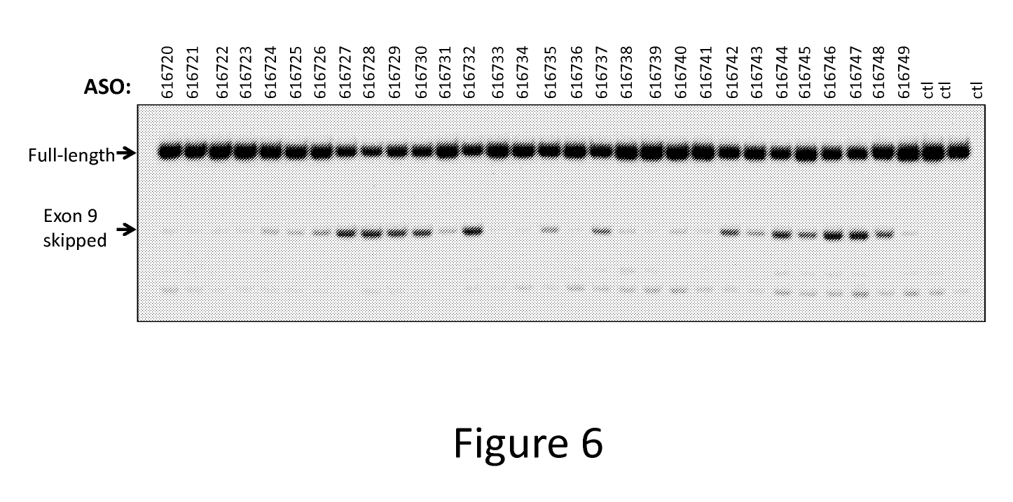

To test the ability of the antisense oligonucleotides to modulate splicing by skipping exon 9, mouse cell line 208ee, derived from C57/Bl6 mouse kidney, was transfected with 50 nM of the ASOs listed in Table 5 using Lipofectamine 2000 (Invitrogen). Untreated controls cells received neither ASO nor Lipofectamine, while mock transfected cells received only Lipofectamine. After 48 hours, total RNA was collected from the cells and RT-PCR was used to identify CLN3 full-length and CLN3Δ9 transcripts. The PCR products were analyzed by PAGE and quantitated by phosphorimager analysis (Typhoon 9400, GE Healthcare). The gel is shown in FIG. 6, and the results are shown in Table 6 as the percentage of exon 9 skipped (Δ9) transcript out of the total full-length (FL) plus exon 9 skipped transcripts. Band intensities are reported in relative fluorescence units (RFU). This example illustrates that many ASOs induce CLN3 exon 9 skipping.

TABLE 6
Induction of CLN3 Exon 9 Skipping in vitro

| | Full-length CLN3 | | Δ9/(FL + Δ9) |
|---|---|---|---|
| ISIS NO | (RFU) | CLN3Δ9 (RFU) | (%) |
| untreated control | 9,181,862 | 123,203 | 1.32 |
| mock | 11,512,749 | 112,061 | 0.96 |
| 616720 | 8,594,979 | 360,150 | 4.02 |
| 616721 | 8,065,276 | 236,557 | 2.85 |
| 616722 | 9,180,522 | 336,620 | 3.54 |
| 616723 | 9,434,304 | 341,373 | 3.49 |
| 616724 | 8,253,838 | 790,632 | 8.74 |
| 616725 | 7,304,964 | 704,209 | 8.79 |
| 616726 | 7,557,368 | 1,175,984 | 13.47 |
| 616727 | 5,415,612 | 3,045,762 | 36.00 |
| 616728 | 4,294,429 | 3,391,413 | 44.13 |
| 616729 | 5,530,907 | 2,760,074 | 33.29 |
| 616730 | 5,419,114 | 2,431,785 | 30.97 |
| 616731 | 8,003,964 | 792,062 | 9.00 |
| 616732 | 5,661,965 | 3,374,571 | 37.34 |
| 616733 | 8,423,448 | 221,903 | 2.57 |
| 616734 | 7,865,161 | 226,765 | 2.80 |
| 616735 | 7,533,174 | 1,036,100 | 12.09 |
| 616736 | 8,566,723 | 261,101 | 2.96 |
| 616737 | 7,177,269 | 1,539,887 | 17.67 |
| 616738 | 9,536,684 | 433,505 | 4.35 |
| 616739 | 10,062,225 | 213,983 | 2.08 |
| 616740 | 9,458,260 | 522,205 | 5.23 |
| 616741 | 9,741,862 | 294,485 | 2.93 |
| 616742 | 6,988,392 | 1,994,800 | 22.21 |
| 616743 | 7,293,927 | 1,125,023 | 13.36 |
| 616744 | 6,063,867 | 2,719,387 | 30.96 |
| 616745 | 7,885,435 | 1,812,122 | 18.69 |
| 616746 | 6,220,132 | 3,850,348 | 38.23 |
| 616747 | 6,181,617 | 3,699,891 | 37.44 |
| 616748 | 7,778,346 | 2,210,461 | 22.13 |
| 616749 | 10,815,904 | 467,192 | 4.14 |

### Example 8: Antisense Oligonucleotide Induction of CLN3 Exon 6 Skipping in Batten Mice In Vivo

A mouse model of Batten Disease, in which the CLN3Δ78 deletion is knocked-in to C57/BL6J mice, has been described (Cotman et al., Hum. Molec. Genet. (2002) 11, 2709-2721). The CLN3Δ78/Δ78 homozygous mice exhibit gliosis in the CNS, abnormal gait traces, clasping behavior indicating neurodegeneration, and early PT-10.

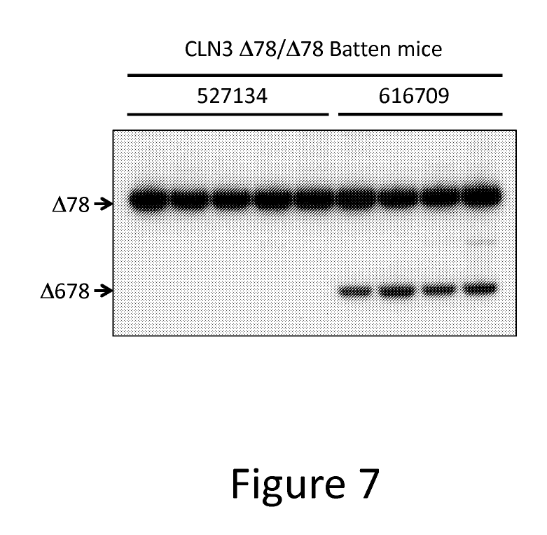

20 μg of Isis No. 616709 (see Table 3) or a control ASO, Isis No. 527134 (AGCTGATCATATTCTACC SEQ ID NO: 61) that is uniform 2′-MOE and contains uniform phosphorothioate internucleoside linkages, was administered to groups of six CLN3Δ78/Δ78 mice to test the ability of Isis No. 616709 to induce exon 6 skipping in vivo. The ASOs were administered by intracerebroventricular injection (ICV injection) on either post-natal day one or two. Four months after the single ICV injection, the mice were euthanized and RNA was isolated from brain tissue. CLN3Δ78 and CLN3Δ678 transcripts were analyzed by radiolabeled RT-PCR. The PCR products were separated by PAGE and quantitated by phosphorimager analysis (Typhoon 9400, GE Healthcare). A gel is shown in FIG. 7, and the results for each group are presented in Table 7 below. As indicated by the results in Table 7 below, ISIS NO. 616709 induced skipping of CLN3 exon 6 in a mouse model of Batten Disease in vivo.

TABLE 7
Induction of CLN3 Exon 6 Skipping in CLN3Δ78/Δ78 mice in vivo

| | Δ678/ | |
|---|---|---|
| | (Δ678 + Δ78) | |
| ISIS NO. | (%) | Std Dev |
| 527134 | 3.70 | 5.30 |
| 616709 | 33.72 | 7.18 |

### Example 9: Antisense Oligonucleotide Rescue of Motor Coordination in CLN3Δ78/Δ78 Mice

In order to test whether ASOs that induce exon 6 skipping relieve symptoms of Batten Disease, homozygous CLN3Δ78/Δ78 and heterozygous CLN3+/Δ78 mice were divided into mixed gender and male only groups and treated with 20 μg of Isis NO. 616709 (SEQ ID NO. 20; see Table 3), control ASO, Isis No. 527134 (SEQ ID NO: 61; see Example 8), or no ASO (untreated). The ASOs were administered by intracerebroventricular injection on post-natal day one or two. Two or three months after the single injection, motor coordination of each mouse was measured based on the time each mouse spent on an accelerating rotarod. The results are presented in Tables 8-12 below.

TABLE 8
Time Spent on an Accelerating Rotarod by Mixed Gender
CLN3Δ78/Δ78 Mice, Age 2 Months

| | | No. | Average Time Spent | |
|---|---|---|---|---|
| | | of Mice in | on Rotarod | |
| Isis No. | Genotype | Group | (seconds) | Std Dev. |
| Untreated | CLN3Δ78/Δ78 | 28 | 82.7 | 25.8 |
| 527134 | CLN3Δ78/Δ78 | 21 | 81.5 | 20.8 |
| 616709 | CLN3Δ78/Δ78 | 21 | 84.7 | 14.5 |
| Untreated | CLN3+/Δ78 | 22 | 91.6 | 19.7 |
| 527134 | CLN3+/Δ78 | 17 | 98.2 | 39.2 |
| 616709 | CLN3+/Δ78 | 31 | 99.8 | 22.9 |

TABLE 9
Time Spent on an Accelerating Rotarod by Mixed Gender
CLN3Δ78/Δ78 Mice, Age 3 Months

| | | No. | Average Time Spent | |
|---|---|---|---|---|
| | | of Mice in | on Rotarod | |
| Isis No. | Genotype | Group | (seconds) | Std Dev. |
| Untreated | CLN3Δ78/Δ78 | 9 | 51.8 | 17.6 |
| 527134 | CLN3Δ78/Δ78 | 8 | 66.1 | 15.3 |
| 616709 | CLN3Δ78/Δ78 | 9 | 81.2 | 12.8 |
| Untreated | CLN3+/Δ78 | 8 | 70.3 | 14.6 |
| 527134 | CLN3+/Δ78 | 7 | 68.8 | 13.4 |
| 616709 | CLN3+/Δ78 | 13 | 96.0 | 29.3 |

TABLE 10
Time Spent on an Accelerating Rotarod by Male
CLN3Δ78/Δ78 Mice, Age 2 Months

| | | No. | Average Time Spent | |
|---|---|---|---|---|
| | | of Mice in | on Rotarod | |
| Isis No. | Genotype | Group | (seconds) | Std Dev. |
| Untreated | CLN3Δ78/Δ78 | 13 | 68.1 | 19.3 |
| 527134 | CLN3Δ78/Δ78 | 5 | 74.3 | 15.2 |
| 616709 | CLN3Δ78/Δ78 | 12 | 84.1 | 14.3 |
| Untreated | CLN3+/Δ78 | 12 | 94.0 | 19.7 |
| 527134 | CLN3+/Δ78 | 8 | 108.2 | 47.5 |
| 616709 | CLN3+/Δ78 | 14 | 103.2 | 23.7 |

TABLE 11
Time Spent on an Accelerating Rotarod by Male
CLN3Δ78/Δ78 Mice, Age 3 Months

| | | No. | Average Time Spent | |
|---|---|---|---|---|
| | | of Mice in | on Rotarod | |
| Isis No. | Genotype | Group | (seconds) | Std Dev. |
| Untreated | CLN3Δ78/Δ78 | 4 | 39.3 | 9.5 |
| 527134 | CLN3Δ78/Δ78 | 1 | 36.4 | n/a |
| 616709 | CLN3Δ78/Δ78 | 4 | 75.3 | 9.9 |
| Untreated | CLN3+/Δ78 | 8 | 70.3 | 14.6 |
| 527134 | CLN3+/Δ78 | 3 | 75.4 | 11.8 |
| 616709 | CLN3+/Δ78 | 7 | 117.4 | 15.8 |

The results in Tables 8-11 above indicate that the motor coordination deficit in the CLN3Δ78/Δ78 Batten mice worsens significantly between two and three months of age and is more severe in males than females. Furthermore, treatment with Isis No. 616709, which induces exon 6 skipping, increases the amount of time CLN3Δ78/Δ78 Batten mice are able to spend on an accelerating rotarod, thereby improving a motor coordination deficit that is symptomatic of Batten Disease.

### Example 10: Antisense Oligonucleotide Induction of Human CLN3 Exon 6 Skipping In Vitro

Antisense oligonucleotides complementary to exon 6 and/or the introns flanking exon 6 of the human CLN3 pre-mRNA transcript were synthesized and tested for their ability to modulate splicing of CLN3 by skipping exon 6. The oligonucleotides in Table 12 below are uniformly 2′-MOE modified, and all internucleoside linkages are phosphorothioate linkages. The cytosine bases are 5-methylcytosine. The ASOs target the complement of GENBANK accession number NT_010393.16, truncated from nucleotides 28427600 to 28444620 (SEQ ID NO: 1), and the ASO sequences are listed in Table 12 below. The start and stop sites associated with each oligonucleotide are the 5′- and 3′-positions, respectively, of the portion of SEQ ID NO: 1 that is complementary to the ASO.

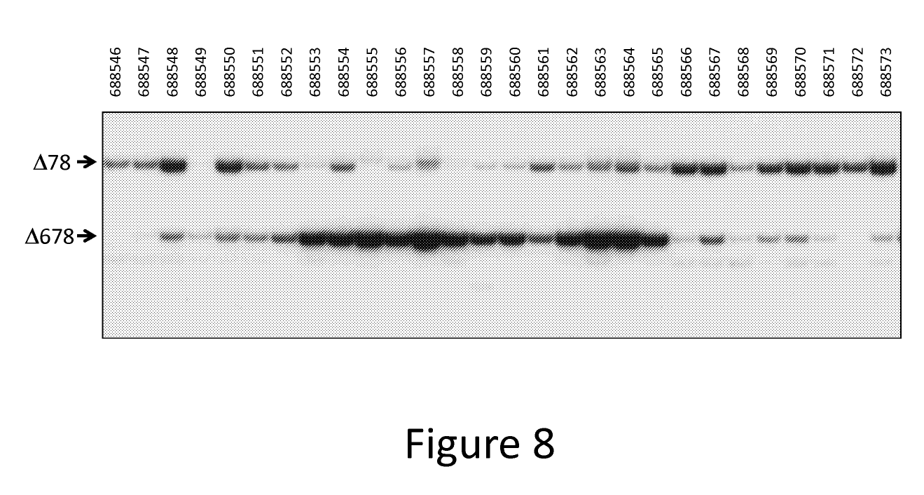

To test the ability of the antisense oligonucleotides to modulate splicing by skipping exon 6, human JNCL patient fibroblasts that are homozygous for the CLN3Δ78 deletion were transfected with 50 nM of an ASO using Cytofectin transfection reagent. After 48 hours, total RNA was collected from the cells and RT-PCR was used to identify the CLN3Δ78 and CLN3Δ678 mRNA transcripts. The PCR products were analyzed by PAGE and quantitated by phosphorimager analysis (Typhoon 9400, GE Healthcare). The gel is shown in FIG. 8, and the results are shown in Table 12 below as the ratios of exon 6, 7, and 8 skipped (Δ678) transcript to exon 7 and 8 skipped (Δ78) transcript. The results below illustrate that many ASOs induced CLN3 exon 6 skipping.

TABLE 12
CLN3 Exon 6 Skipping in patient fibroblasts

| ISIS | Start | Stop | | CLN3 Target | CLN3Δ678/ | SEQ |
|---|---|---|---|---|---|---|
| No. | Site | Site | Sequence (5′ to 3′) | Region | CLN3Δ78 | ID NO |
| 688546 | 5699 | 5716 | ACTCTCAGCATCTCAGCC | Intron 5 | 0.00 | 63 |
| 688547 | 5704 | 5721 | TCTCTACTCTCAGCATCT | Intron 5 | 0.00 | 64 |
| 688548 | 5709 | 5726 | GTCGGTCTCTACTCTCAG | Intron 5 | 0.25 | 65 |
| 688549 | 5714 | 5731 | GGAAGGTCGGTCTCTACT | Intron 5 | 1.38 | 66 |
| 688550 | 5734 | 5751 | GGTGAGAAGGGAAGGGAG | Intron 5 | 0.37 | 67 |
| 688551 | 5764 | 5781 | TCCCACTGACGAGAACCC | Exon 6 | 0.74 | 68 |
| 688552 | 5769 | 5786 | ACAAATCCCACTGACGAG | Exon 6 | 1.58 | 69 |
| 688553 | 5774 | 5791 | GCAGCACAAATCCCACTG | Exon 6 | 14.77 | 70 |
| 688554 | 5779 | 5796 | TTCCAGCAGCACAAATCC | Exon 6 | 3.68 | 71 |
| 688555 | 5784 | 5801 | GAAGCTTCCAGCAGCACA | Exon 6 | 31.07 | 72 |
| 688556 | 5789 | 5806 | AGGACGAAGCTTCCAGCA | Exon 6 | 9.28 | 73 |
| 688557 | 5794 | 5811 | CAACCAGGACGAAGCTTC | Exon 6 | 13.03 | 74 |
| 688558 | 5799 | 5816 | AAAGGCAACCAGGACGAA | Exon 6 | 24.37 | 75 |
| 688559 | 5804 | 5821 | TGAGAAAAGGCAACCAGG | Exon 6 | 10.29 | 76 |
| 688560 | 5809 | 5826 | CAGAATGAGAAAAGGCAA | Exon 6 | 8.44 | 77 |
| 688561 | 5814 | 5831 | CCCCACAGAATGAGAAAA | Exon 6 | 1.28 | 78 |
| 688562 | 5819 | 5836 | CTGGTCCCCACAGAATGA | Exon 6 | 7.32 | 79 |
| 688563 | 5824 | 5841 | ACAGGCTGGTCCCCACAG | Exon 6 | 8.32 | 80 |
| 688564 | 5829 | 5846 | ACCACACAGGCTGGTCCC | Exon 6/Intron 6 | 6.52 | 81 |
| 688565 | 5834 | 5851 | CACTCACCACACAGGCTG | Exon 6/Intron 6 | 4.56 | 82 |
| 688566 | 5839 | 5856 | CCACACACTCACCACACA | Exon 6/Intron 6 | 0.16 | 83 |
| 688567 | 5844 | 5861 | CAGAACCACACACTCACC | Intron 6 | 0.41 | 84 |
| 688568 | 5849 | 5866 | TGACACAGAACCACACAC | Intron 6 | 0.46 | 85 |
| 688569 | 5854 | 5871 | CCATCTGACACAGAACCA | Intron 6 | 0.33 | 86 |
| 688570 | 5859 | 5876 | GCTCCCCATCTGACACAG | Intron 6 | 0.27 | 87 |
| 688571 | 5879 | 5896 | CTCTGATGTGGTTCCTCG | Intron 6 | 0.11 | 88 |
| 688572 | 5884 | 5901 | AAATGCTCTGATGTGGTT | Intron 6 | 0.10 | 89 |
| 688573 | 5889 | 5906 | CCCACAAATGCTCTGATG | Intron 6 | 0.12 | 90 |

### Example 11: Antisense Oligonucleotide Induction of Human CLN3 Exon 9 Skipping In Vitro

Antisense oligonucleotides complementary to exon 9 and/or the introns flanking exon 9 of the human CLN3 pre-mRNA transcript were synthesized and tested for their ability to modulate splicing of CLN3 by skipping exon 9. The oligonucleotides were uniformly modified to contain 2′-MOE, and all internucleoside linkages are phosphorothioate linkages. The cytosine bases are 5-methylcytosine. The ASOs target the complement of GENBANK accession number NT_010393.16, truncated from nucleotides 28427600 to 28444620 (SEQ ID NO: 1), and the ASO sequences are listed in Table 13 below. The start and stop sites associated with each oligonucleotide are the 5′- and 3′-positions, respectively, of the portion of SEQ ID NO: 1 that is complementary to the ASO.

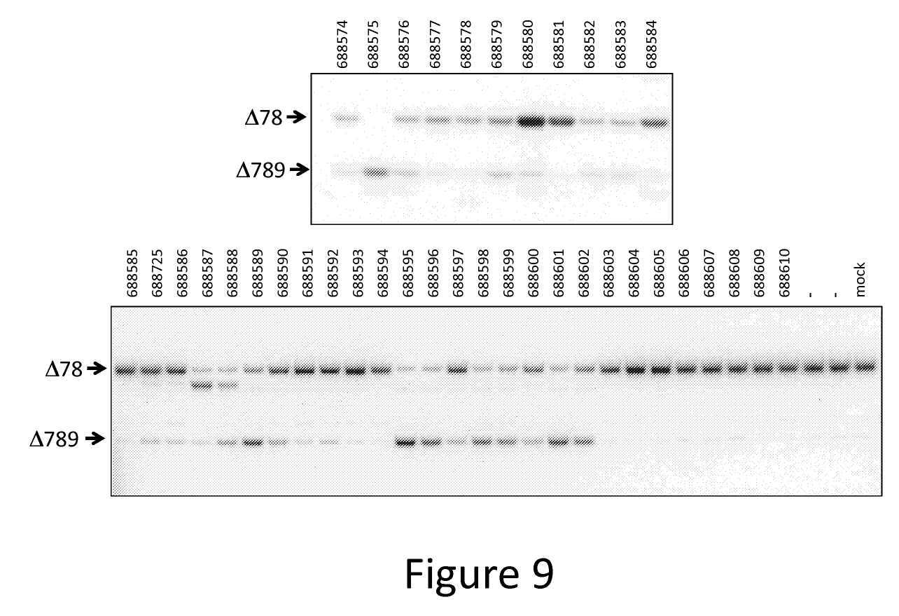

To test the ability of the antisense oligonucleotides to modulate splicing by skipping exon 9, human JNCL patient fibroblasts that are homozygous for the CLN3Δ78 deletion were transfected with 50 nM of an ASO using Cytofectin transfection reagent or were mock transfected. After 48 hours, total RNA was collected from the cells and RT-PCR was used to identify the CLN3Δ78 and CLN3Δ789 mRNA transcripts. The PCR products were analyzed by PAGE and quantitated by phosphorimager analysis (Typhoon 9400, GE Healthcare). The gel is shown in FIG. 9, and the results are shown in Table 13 below as the ratios of exon 7, 8, and 9 skipped (Δ789) transcript to exon 7 and 8 skipped (Δ78) transcript. The results below illustrate that many ASOs induced CLN3 exon 9 skipping to a greater extent than the exon 9 skipping observed in mock transfected cells.

TABLE 13
CLN3 Exon 9 Skipping in patient fibroblasts

| ISIS | Start | Stop | | CLN3 Target | CLN3Δ789/ | SEQ |
|---|---|---|---|---|---|---|
| No. | Site | Site | Sequence (5′ to 3′) | Region | CLN3Δ78 | ID NO |
| mock | n/a | n/a | n/a | n/a | 0.05 | n/a |
| 688574 | 9122 | 9139 | GGGAAGGTCCCCAGGGAC | Intron 8 | 1.14 | 91 |
| 688575 | 9127 | 9144 | TGGCTGGGAAGGTCCCCA | Intron 8 | 48.56 | 92 |
| 688576 | 9132 | 9149 | CCTACTGGCTGGGAAGGT | Intron 8 | 1.59 | 93 |
| 688577 | 9137 | 9154 | AAAACCCTACTGGCTGGG | Intron 8 | 0.46 | 94 |
| 688578 | 9142 | 9159 | GTCAGAAAACCCTACTGG | Intron 8 | 0.46 | 95 |
| 688579 | 9147 | 9164 | GCAGGGTCAGAAAACCCT | Intron 8 | 0.89 | 96 |
| 688580 | 9152 | 9169 | TGAAGGCAGGGTCAGAAA | Intron 8 | 0.20 | 97 |
| 688581 | 9157 | 9174 | TAGGATGAAGGCAGGGTC | Intron 8 | 0.08 | 98 |
| 688582 | 9162 | 9179 | AGGAGTAGGATGAAGGCA | Intron 8 | 0.79 | 99 |
| 688583 | 9167 | 9184 | TAGCTAGGAGTAGGATGA | Intron 8/Exon 9 | 1.05 | 100 |
| 688584 | 9172 | 9189 | AGAAATAGCTAGGAGTAG | Intron 8/Exon 9 | 0.22 | 101 |
| 688585 | 9177 | 9194 | CAACAAGAAATAGCTAGG | Intron 8/Exon 9 | 0.08 | 102 |
| 616725 | 9182 | 9199 | GTGAGCAACAAGAAATAG | Exon 9 | 0.29 | 36 |
| 688586 | 9187 | 9204 | GAGATGTGAGCAACAAGA | Exon 9 | 0.24 | 103 |
| 688587 | 9192 | 9209 | CTCAGGAGATGTGAGCAA | Exon 9 | 0.92 | 104 |
| 688588 | 9197 | 9214 | TGGGCCTCAGGAGATGTG | Exon 9 | 2.73 | 105 |
| 688589 | 9202 | 9219 | GGTCCTGGGCCTCAGGAG | Exon 9 | 2.73 | 106 |
| 688590 | 9207 | 9224 | TCCAGGGTCCTGGGCCTC | Exon 9 | 0.57 | 107 |
| 688591 | 9212 | 9229 | TCCCCTCCAGGGTCCTGG | Exon 9 | 0.14 | 108 |
| 688592 | 9217 | 9234 | CTTCTTCCCCTCCAGGGT | Exon 9 | 0.23 | 109 |
| 688593 | 9222 | 9239 | TGCTTCTTCTTCCCCTCC | Exon 9 | 0.06 | 110 |
| 688594 | 9227 | 9244 | CTCTCTGCTTCTTCTTCC | Exon 9 | 0.07 | 111 |
| 688595 | 9232 | 9249 | CTGCGCTCTCTGCTTCTT | Exon 9 | 8.80 | 112 |
| 688596 | 9237 | 9254 | CCGGGCTGCGCTCTCTGC | Exon 9 | 4.76 | 113 |
| 688597 | 9242 | 9259 | GGCTGCCGGGCTGCGCTC | Exon 9 | 0.51 | 114 |
| 688598 | 9262 | 9279 | GGGCCTCGGTTCTTATGA | Exon 9 | 3.74 | 115 |
| 688599 | 9282 | 9299 | CCTACCTGGCTTCGACTC | Exon 9/Intron 9 | 2.30 | 116 |
| 688600 | 9287 | 9304 | TGTCTCCTACCTGGCTTC | Exon 9/Intron 9 | 0.79 | 117 |
| 688601 | 9292 | 9309 | GTCTGTGTCTCCTACCTG | Exon 9/Intron 9 | 5.10 | 118 |
| 688602 | 9297 | 9314 | TGAGGGTCTGTGTCTCCT | Intron 9 | 1.75 | 119 |
| 688603 | 9302 | 9319 | CTCTCTGAGGGTCTGTGT | Intron 9 | 0.08 | 120 |
| 688604 | 9307 | 9324 | GTGACCTCTCTGAGGGTC | Intron 9 | 0.03 | 121 |
| 688605 | 9312 | 9329 | AGAAAGTGACCTCTCTGA | Intron 9 | 0.05 | 122 |
| 688606 | 9317 | 9334 | GAGAAAGAAAGTGACCTC | Intron 9 | 0.07 | 123 |
| 688607 | 9322 | 9339 | CCAGAGAGAAAGAAAGTG | Intron 9 | 0.05 | 124 |
| 688608 | 9327 | 9344 | CAAACCCAGAGAGAAAGA | Intron 9 | 0.08 | 125 |
| 688609 | 9332 | 9349 | AAGGCCAAACCCAGAGAG | Intron 9 | 0.02 | 126 |
| 688610 | 9337 | 9354 | AGGAAAAGGCCAAACCCA | Intron 9 | 0.01 | 127 |

### Example 12: Dose Response of Antisense Oligonucleotide Induction of Human CLN3 Exon Skipping

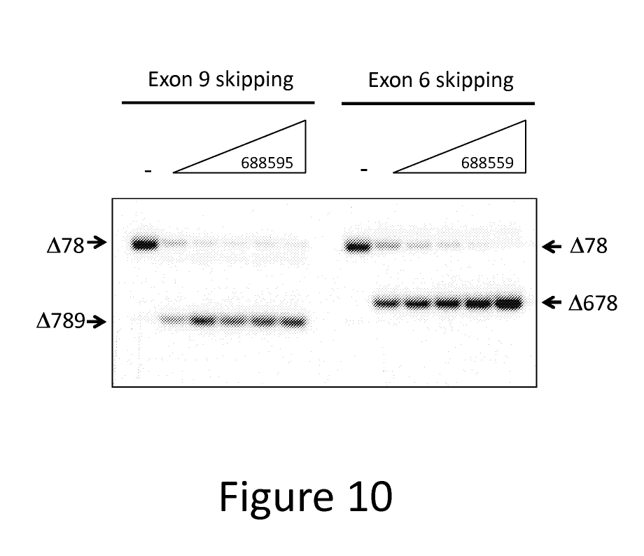

JNCL patient fibroblasts were transfected with Isis No. 688555, 688559 (see Table 12), or Isis No. 688595 (see Table 13) at a concentration listed in Table 14 below using Endo-porter transfection reagent (Gene Tools, LLC). After 48 hours, total RNA was isolated from the cells and CLN3 mRNA was detected as described in Examples 10 and 11. The gel is shown in FIG. 10. The results are shown in Table 14 below and show that all three ASOs tested induced exon skipping in a dose dependent manner. “n/a” indicates that the ASO was not tested at the indicated concentration.

TABLE 14
Dose Responses of an Exon 6 Skipping ASO and an Exon 9
Skipping ASO in Patient Cells

| | | | CLN3Δ789/ |
|---|---|---|---|
| Concentration | CLN3Δ678/CLN3Δ78 | | CLN3Δ78 |
| (nM) | Isis No. 688555 | Isis No. 688559 | Isis No. 688595 |
| 0 | 0.00 | 0.01 | 0.04 |
| 0.39 | 0.37 | n/a | n/a |
| 0.78 | 1.13 | n/a | n/a |
| 1.56 | 4.31 | n/a | n/a |
| 3.125 | 15.24 | n/a | n/a |
| 12.5 | n/a | 3.46 | 3.84 |
| 25 | n/a | 7.36 | 19.34 |
| 50 | n/a | 11.88 | 23.16 |
| 100 | n/a | 23.66 | 21.31 |
| 200 | n/a | 70.91 | 27.9 |

### Example 13: Performance in a Water Maze by CLN3Δ78/Δ78 Mice Following ASO Treatment

In order to test whether an ASO that induces exon 6 skipping relieves symptoms of Batten Disease, homozygous CLN3Δ78/Δ78, heterozygous CLN3+/Δ78, and wild type mice were treated with 20 μg of Isis No. 616709 (see Table 3), Isis No. 527134 as a control (see Example 8), or no ASO (untreated). The ASOs were administered by intracerebroventricular injection on post-natal day one or two. Two or three months after the single injection, performance in the Morris water maze was measured based on the time each mouse took to swim to a hidden platform. Mice were placed individually in a circular pool, 48 inches in diameter, filled to a depth of 26 inches with 23° C. water. The pool walls were made opaque with white paint and placed in a room with prominent extra-maze cues at least 16 inches from the pool edge. Four unique, proximal cues were affixed to the 8 cm high interior pool wall above water level at 0, 90, 180, and 270 degrees. Mice were placed in one of four starting quadrants facing the pool wall and allowed to swim until coming to rest atop a 4 inch square plexiglass platform submerged in 0.5 cm of water, or until a maximum of 60 seconds. Upon finding a platform, mice were left there for 20 seconds before reentry at the next start point or removal from the cage. Mice that did not find the platform within 60 seconds were guided to it by the experimenter. Trials were performed once at each starting quadrant point per session. Mice were tested for four consecutive days with two sessions of four trials each per day, and the time to reach a hidden platform was recorded. Each treatment group consisted of 13-35 mice. The results for each treatment group are shown in Table 15 below. The results show that CLN3Δ78/Δ78 homozygous mice that received an antisense oligonucleotide that induces exon 6 skipping performed similarly to WT or heterozygous mice and performed significantly better than CLN3Δ78/Δ78 homozygous mice that were untreated or received the control ASO. In particular, the differences between the control CLN3Δ78/Δ78 homozygous mice and the ASO treated CLN3Δ78/Δ78 homozygous mice and WT and heterozygous controls were significant, with p values ≦0.01 on days 2 and 4.

TABLE 15
Time to Reach a Hidden Platform in a Morris Water Maze

| | | Number of | Trial | Time to | Std. |
|---|---|---|---|---|---|
| Isis No. | Genotype | mice in group | day | Platform(s) | Dev. |
| Untreated | WT or | 21 | 1 | 44.8 | 10.5 |
| | CLN3+/Δ78 | | 2 | 24.9 | 12.0 |
| | | | 3 | 17.2 | 8.3 |
| | | | 4 | 13.5 | 7.6 |
| Untreated | CLN3Δ78/Δ78 | 35 | 1 | 48.9 | 10.0 |
| or | | | 2 | 35.9 | 14.5 |
| 527134 | | | 3 | 27.4 | 12.7 |
| | | | 4 | 26.0 | 14.2 |
| 616709 | CLN3Δ78/Δ78 | 13 | 1 | 43.0 | 7.6 |
| | | | 2 | 19.3 | 7.5 |
| | | | 3 | 24.1 | 12.9 |
| | | | 4 | 16.8 | 8.4 |

1.-225. (canceled)

226. A compound comprising a modified oligonucleotide consisting of 8 to 30 linked nucleosides and having a nucleobase sequence comprising a complementary region, wherein the complementary region comprises at least 8 contiguous nucleobases and is complementary to an equal-length portion of a target region of a CLN3 transcript.

227. The compound of claim 226, wherein the target region of the CLN3 transcript comprises at least a portion of exon 6 of the CLN3 transcript.

228. The compound of claim 226, wherein the target region of the CLN3 transcript comprises at least a portion of exon 9 of the CLN3 transcript.

229. The compound of claim 226, wherein the target region of the CLN3 transcript comprises at least a portion of intron 5 of the CLN3 transcript.

230. The compound of claim 226, wherein the target region of the CLN3 transcript comprises at least a portion of intron 6 of the CLN3 transcript.

231. The compound of claim 226, wherein the target region of the CLN3 transcript comprises at least a portion of intron 9 of the CLN3 transcript.

232. The compound of claim 226, wherein the target region of the CLN3 transcript comprises at least a portion of intron 10 of the CLN3 transcript.

233. The compound of claim 226, wherein the complementary region of the modified oligonucleotide is 100% complementary to the target region.

234. The compound of claim 226, wherein the nucleobase sequence of the oligonucleotide is at least 90% complementary to an equal-length region of the CLN3 transcript, as measured over the entire length of the oligonucleotide.

235. The compound of claim 226, wherein the nucleobase sequence of the antisense oligonucleotide comprises any one of SEQ ID NOs: 3 to 60.

236. The compound of claim 226, wherein the modified oligonucleotide comprises at least one modified nucleoside.

237. The compound of claim 236, wherein at least one modified nucleoside comprises a modified sugar moiety.

238. The compound of claim 237, wherein at least one modified sugar moiety is a 2′-substituted sugar moiety.

239. The compound of claim 238, wherein the 2′-substitutent of at least one 2′-substituted sugar moiety is selected from among: 2′-OMe, 2′-F, and 2′-MOE.

240. The compound of claim 239, wherein the 2′-substituent of at least one 2′-substituted sugar moiety is a 2′-MOE.

241. The compound of claim 226, wherein at least one modified sugar moiety is a bicyclic sugar moiety.

242. A method of modulating splicing of a CLN3 transcript in a cell comprising contacting the cell with a compound according to claim 226.

243. The method claim 242, wherein the amount of CLN3 mRNA without exon 6 is increased.

244. The method of claim 242, wherein the amount of CLN3 mRNA without exon 9 is increased.

245. The method of claim 242, wherein the amount of CLN3 mRNA with exon 10 is increased.
